SEQUENCE LISTING (140 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, Artificial Sequence
  Description of Artificial Sequence Synthetic polypeptide
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, Artificial Sequence
  Description of Artificial Sequence Synthetic peptide
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, Artificial Sequence [sequence omitted]

SEQ ID NO: 81 - protein, 108 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, Artificial Sequence [sequence omitted]

SEQ ID NO: 90 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, Artificial Sequence [sequence omitted]

SEQ ID NO: 114 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, Artificial Sequence
  Description of Artificial Sequence Synthetic polypeptide
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, Artificial Sequence
  Description of Artificial Sequence Synthetic polypeptide
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

(19) United States
(12) Patent Application Publication
(10) Pub. No.: US 2022/0281987 A1
(43) Pub. Date: Sep. 8, 2022

(54) METHODS FOR THE TREATMENT OF GPP

(71) Applicant: Boehringer Ingelheim International GmbH, Ingelheim am Rhein (DE)

(72) Inventors: David B. HALL, Ovid, NY (US); Benjamin LANG, Wangen (DE); Christian THOMA, Biberach an der Riss (DE)

(21) Appl. No.: 17/685,423

(22) Filed: Mar. 3, 2022

Related U.S. Application Data
(60) Provisional application No. 63/287,150, filed on Dec. 8, 2021.
(60) Provisional application No. 63/237,672, filed on Aug. 27, 2021.
(60) Provisional application No. 63/178,007, filed on Apr. 22, 2021.
(60) Provisional application No. 63/156,600, filed on Mar. 4, 2021.

(51) Int. Cl. C07K 16/28; A61P 17/06

(52) U.S. Cl. CPC C07K 16/2866; A61P 17/06; A61K 2039/54

(57) ABSTRACT

The present invention relates to the treatment of or alleviation of signs and symptoms of GPP or an acute phase flare-up of generalized pustular psoriasis (GPP) with anti-IL36R antibodies.

### SEQUENCE LISTING

The instant application contains a Sequence Listing which has been submitted in ASCII format via EFS-Web and is hereby incorporated by reference in its entirety. Said ASCII copy, created on Jan. 31, 2022, is named 09-0719-US-5-2022-03-03-SL.txt and is 147,456 bytes in size.

### TECHNICAL FIELD OF THE INVENTION

The present invention relates to use of anti-IL-36R antibodies in methods and compositions for treatment of patients with generalized pustular psoriasis (GPP). More specifically, the invention relates to the treatment of GPP or GPP flares in a patient by administering to the patient two 900 mg intravenous (i.v.) doses of an anti-IL-36R antibody; wherein the second dose is administered less than 2 weeks after the first dose. More specifically, the invention relates to the treatment of GPP in a patient by administering to the patient a single 900 mg intravenous dose of spesolimab, if the GPP symptoms persist, administering an additional 900 mg intravenous dose one week after the initial dose.

### BACKGROUND

GPP is a severe skin disease characterized by the repeated occurrence of acute flares caused by systemic inflammation affecting the skin and internal organs. The classic presentation of acute GPP was first described as a recurrent pustular form of psoriasis by von Zumbusch in 1909. While GPP and plaque psoriasis can occur at the same time in an individual patient, GPP is distinct from plaque psoriasis in clinical presentation, pathophysiology, histopathology, response to therapies, epidemiology and genetics.

Therefore, it is very critical to differentiate GPP from plaque or erythrodermic psoriasis with secondary pustulation. The clinical presentation of GPP is quite different from psoriasis vulgaris (PV) in its' episodic nature, often with normal appearing skin between very acute and severe disease flares. GPP is clinically characterized by the preponderance of pustules as the primary lesion on an erythematous base rather than red plaques covered with silvery scales representing the primary lesion of typical plaque psoriasis. In addition, the histopathological hallmarks of GPP are distinct spongiform pustules of Kogoj located in the subcorneal portion of the epidermis. GPP may be associated with systemic symptoms (fever, increased CRP and neutrophilia) and severe extra-cutaneous organ manifestations (liver, kidney failure, CV shock). While patients with GPP may have pre-existing or co-existing PV, it is possible to clinically distinguish patients with primary plaque disease (PV) who have a secondary pustular component from patients who have primary pustular disease (GPP) with a concomitant plaque component, based on the sequence of manifestations (primary lesion pustule rather than plaque) and the localization of a GPP pustule on an erythematous base rather than a PsO plaque.

As descriptions for GPP are discordant among standard dermatology textbooks, the European Rare And Severe Psoriasis Expert Network (ERASPEN) has defined consensus criteria that include as key diagnosis criteria for acute GPP the presence of primary, sterile, macroscopically visible pustules on non-acral skin (excluding cases where pustulation is restricted to psoriatic plaques), with or without systemic inflammation, with or without plaque-type psoriasis, either relapsing (>1 episode) or persistent (>3 months).

Chronic GPP describes the state in between disease flares that may be characterized by the complete absence of symptoms or the persistence of residual skin symptoms such as erythema and scaling and minor pustulation.

Current treatment options for controlling acute GPP and subcutaneous of response are limited and do not provide sustained efficacy. No treatments are currently approved for GPP in the US and EU, though retinoids, cyclosporine or methotrexate are being recommended. Although these treatments are described to be “remarkably effective or effective” in 70-84% of patients (J Am Acad Dermatol. 2012; 67(2):279-88) these data are based on a retrospective cohort study from Japan without clearly defined endpoints (Japanese Journal of Dermatology. 2010; 120(4):815-39). Furthermore, these treatments cannot be used long-term due to side effects and contraindications (retinoids: teratogenicity, hair loss; cyclosporine: excessive hair growth, renal toxicity; MTX: liver toxicity).

Biologics (mostly TNF inhibitors, occasionally IL-1 or IL-17 inhibitors) are increasingly used to treat more severe, extensive or treatment resistant patients with GPP, based on small published case series. However, these drugs are also associated with limitations in efficacy (incomplete and delayed responses are frequent) and safety as well as contraindications (infusion reactions, tuberculosis, cardiovascular disease). Thus, a need exists in the art for novel targeted therapies for the treatment and/or prevention of GPP.

### SUMMARY OF THE INVENTION

The present invention addresses the above need by providing biotherapeutics, in particular antibodies, which bind to IL-36R and provide therapeutic or prophylactic therapy for GPP including acute GPP and the associated signs and symptoms such as GPP flares.

In one aspect, the invention relates to the treatment of GPP in a patient by administering to the patient two 900 mg intravenous (i.v.) doses of an anti-IL-36R antibody; wherein the second dose is administered less than 2 weeks after the first dose.

In another aspect, the invention relates to the treatment of GPP in a patient by administering to the patient a single 900 mg intravenous dose of an anti-IL-36R antibody, if the GPP symptoms persist, administering an additional 900 mg intravenous dose an anti-IL-36R antibody one week after the initial dose.

In one aspect, the invention relates to a method of treating generalized pustular psoriasis (GPP) flares in a patient, said method comprising administering to the patient two 900 mg intravenous (i.v.) doses of an anti-IL-36R antibody; wherein the second dose is administered less than 2 weeks after the first dose.

In one aspect, the invention relates to a method of treating GPP in a patient, said method comprising administering to the patient two 900 mg intravenous doses of an anti-IL-36R antibody; wherein the second dose is administered less than 2 weeks after the first dose.

In one aspect, the invention relates to a method of reducing or alleviating signs and symptoms of an acute phase flare-up of GPP in a patient, said method comprising administering to the patient two 900 mg intravenous doses of an anti-IL-36R antibody; wherein the second dose is administered less than 2 weeks after the first dose.

In one aspect, the invention relates to a method of reducing the severity and duration of GPP symptoms, said method comprising including administering to the patient two 900 mg intravenous doses of an anti-IL-36R antibody; wherein the second dose is administered less than 2 weeks after the first dose.

In one aspect, the invention relates to a method of treating a skin disorder associated with GPP, said method comprising administering to the patient two 900 mg intravenous doses of an anti-IL-36R antibody; wherein the second dose is administered less than 2 weeks after the first dose.

In one aspect, the invention relates to a method of preventing the recurrence of GPP flares in a patient, said method comprising administering to the patient two 900 mg intravenous doses of an anti-IL-36R antibody; wherein the second dose is administered less than 2 weeks after the first dose.

In one aspect, the invention relates to a method of reducing pain by at least 10% in a patient with GPP, said method comprising administering to the patient two 900 mg intravenous doses of an anti-IL-36R antibody; wherein the second dose is administered less than 2 weeks after the first dose.

In one aspect, the invention relates to a method of improving the quality of life by at least 10% in a patient with moderate to severe GPP symptoms, said method comprising administering to the patient two 900 mg intravenous doses of an anti-IL-36R antibody; wherein the second dose is administered less than 2 weeks after the first dose.

In an embodiment relating to any of the above aspects, the patient has a GPP Physician Global Assessment (GPPGA) total score of ≥2.

In an embodiment relating to any of the above aspects, the patient has a GPP Physician Global Assessment (GPPGA) total score of ≥2 and a GPPGA pustulation subscore of ≥2.

In an embodiment relating to any of the above aspects, the patient has a GPP Physician Global Assessment (GPPGA) total score of ≥2 and a GPPGA pustulation subscore of ≥2 before the administration of the first i.v. dose.

In an embodiment relating to any of the above aspects, the patient has a GPP Physician Global Assessment (GPPGA) total score of ≥2 and a GPPGA pustulation subscore of ≥2 after the administration of the first i.v. dose.

In an embodiment relating to any of the above aspects, the patient has a GPP Physician Global Assessment (GPPGA) total score of ≥2 and a GPPGA pustulation subscore of ≥2 before and after the administration of the first i.v. dose.

In an embodiment relating to any of the above aspects, the second dose is administered after 1 week but less than 2 weeks from the first dose.

In one aspect, the invention relates to a method of treating a GPP patient with a GPPGA pustulation subscore of ≥2, said method comprising the steps of: (a) administering to the patient a first 900 mg intravenous (I.V.) dose of an anti-IL-36R antibody; (b) assessing the GPPGA pustulation subscore of the patient and if the GPPGA pustulation subscore of ≥2 of the patient persists after 1 week from the first dose, then administering to the patient a second 900 mg (i.v.) dose of spesolimab less than 2 weeks after the first dose.

In one aspect, the invention relates to a method of treating a GPP patient with a GPP Physician Global Assessment (GPPGA) total score of ≥2, said method comprising the steps of: (a) administering to the patient a first 900 mg intravenous (i.v.) dose of an anti-IL-36R antibody; (b) assessing the GPPGA total score of the patient and if the GPPGA total score of ≥2 of the patient persists after 1 week from the first dose, then administering to the patient a second 900 mg (i.v.) dose of spesolimab less than 2 weeks after the first dose.

In one aspect, the invention relates to a method of treating a GPP patient with a GPP Physician Global Assessment (GPPGA) total score of ≥2 and a GPPGA pustulation subscore of ≥2, said method comprising the steps of: (a) administering to the patient a first 900 mg intravenous (i.v.) dose of an anti-IL-36R antibody; (b) assessing the GPPGA scores of the patient and if the GPPGA total score of ≥2 and the GPPGA pustulation subscore of ≥2 of the patient persist after 1 week from the first dose, then administering to the patient a second 900 mg (i.v.) dose of spesolimab less than 2 weeks after the first dose.

In an embodiment relating to any of the above aspects or embodiments, an optional third 900 mg i.v. dose of the anti-IL-36R antibody is administered at 2 to 12 weeks after the second i.v. dose.

In an embodiment relating to any of the above aspects or embodiments, the two-dose administration achieves one or more of the following results: (a) a Generalized Pustular Psoriasis Global Assessment (GPPGA) pustulation subscore of 0 indicating in one week after administering the second i.v. dose; and/or (b) a GPPGA total score of 0 or 1 in one week after administering the second i.v. dose.

In an embodiment relating to any of the above aspects or embodiments, the results are maintained for up to and at least 12 weeks following the administration of the second i.v. dose.

In an embodiment relating to any of the above aspects or embodiments, the method comprises administering to the patient a prophylactically effective amount of the anti-IL-36R antibody in one or more subcutaneous doses after the last i.v. dose administered.

In an embodiment relating to any of the above aspects or embodiments, each of the one or more subcutaneous doses comprises 150 mg, 225 mg, 300 mg, 450 mg, or 600 mg of said anti-IL-36R antibody.

In an embodiment relating to any of the above aspects or embodiments, 1, 2, 3 or more subcutaneous doses are administered to the patient and wherein a first subcutaneous dose is administered after the last intravenous dose.

In an embodiment relating to any of the above aspects or embodiments, a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.

In an embodiment relating to any of the above aspects or embodiments, the patient remains in clinical remission as measured by a GPPGA total score of 0 or 1 for at least 12, 24, 36, 48, 60 or 72 weeks following the last i.v. or subcutaneous dose.

In an embodiment relating to any of the above aspects or embodiments, the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In an embodiment relating to any of the above aspects or embodiments, the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In an embodiment relating to any of the above aspects or embodiments, the anti-IL-36R antibody comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects or embodiments, the anti-IL-36R antibody comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the above aspects or embodiments, the anti-IL-36R antibody is spesolimab

Additional features and advantages of the present invention will be set forth in the description below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE DRAWINGS

The accompanying drawings, which are included to provide further understanding of the present invention and are incorporated in and constitute a part of this specification, illustrate aspects of the subject technology and together with the description serve to explain the principles of the present invention.

FIG. 1 shows a CONSORT flow diagram for the trial described in Example 1. *Exclusion by other include trial completion, global recruitment target achieved and patients who did not flared within the 6-month screening period. †Patients were blinded to randomized treatment, but could be eligible to an open-label dose of spesolimab at Day 8. †Patients who did not complete 16 weeks residual period after last spesolimab dose were not rolled over into the OLE trial. Patients who did not continue in the OLE trial were to be followed for 16 weeks after the last dose of trial medication, which is the latest timepoint of trial medication given during the trial (e.g. day 1, day 8 if OL spesolimab is given, rescue with OL spesolimab if given). OL, open-label; OLE, open-label extension; SoC, standard of care.

FIG. 2 shows the trial design as described in Example 1. *Day 2-7: Escape treatment (SoC) may be offered in case of disease worsening defined as worsening of clinical status or GPP skin and/or systemic symptoms as defined by the investigator. †After day 8—week 12: only one rescue dose with OL spesolimab is permitted if a patient who has previously achieved clinical response (GPPGA 0/1) to initial treatment, either with spesolimab or placebo at day 1, or escape medication or OL spesolimab at day 8, experiences a recurrence of a GPP flare (2-point increase in the GPPGA score and the pustular component of GPPGA ≥2). Subsequent flares will be treated with SoC per physician's choice. †Patients who do not require rescue treatment with OL spesolimab are to be followed until week 12 (EoT) prior to entering into OLE trial. Patients who receive rescue treatment with OL spesolimab between weeks 2 and week 6 are to be followed until week 12 (EoT) prior to entering the OLE trial. If at week 12 they qualify to enter the OLE trial, then the EoT will be considered for these patients. Patients who do not qualify to enter the OLE trial are to be followed for 16 weeks (EoT/week 16-28) after the last dose of trial medication, which is the latest timepoint of trial medication given during the trial (e.g. day 1, day 8 if OL spesolimab is given, rescue with OL spesolimab if given). EoT, end of trial; GPP, generalized pustular psoriasis; GPPGA, Generalized Pustular Psoriasis Physician Global Assessment; i.v., intravenous; OL, open-label, OLE, open-label extension; R, randomisation; SD, single dose; SoC, standard of care.

FIGS. 3A and 3B shows the primary and key secondary outcomes. FIG. 3A shows the percentage patients who had a Generalized Pustular Psoriasis Physician Global Assessment (GPPGA) pustulation subscore of 0 (complete pustule clearance) at week 1 after spesolimab or placebo treatment. FIG. 3B shows the percentage patients who had global GPPGA score of 0 or 1 (clear or almost clear skin) at week 1 after a single intravenous dose of 900 mg spesolimab or placebo treatment. CI, confidence interval.

FIGS. 4A and 4B shows GPPGA Pustulation Subscore Over Time by Randomized Treatment at Day 1. FIGS. 4A and 4 B show the proportion of patients by Generalized Pustular Psoriasis Physician Global Assessment (GPPGA) pustulation subscores at week 1 (day 8) and over time after receiving a single intravenous dose of placebo (FIG. 4A) or 900 mg spesolimab (FIG. 4B), respectively. Dashed line represents proportion of patients achieving GPPGA pustulation subscore of 0. *For this analysis, any use of escape medication, or open-label spesolimab at day 8, or rescue medication with open-label spesolimab at each timepoint were categorized separately (gray bars).

FIGS. 5A and 5B shows GPPGA Total Score Over Time by Randomized Treatment at Day 1. FIGS. 5A and 5B show the proportion of patients by Generalized Pustular Psoriasis Physician Global Assessment (GPPGA) total score at week 1 (day 8) and over time after receiving a single intravenous dose of placebo (FIG. 5A) or 900 mg spesolimab (FIG. 5B), respectively. Dashed line represents proportion of patients achieving GPPGA Total score of 0 or 1. *For this analysis, any use of escape medication, or open-label spesolimab at day 8, or rescue medication with open-label spesolimab at each timepoint were categorized separately (gray bars).

FIG. 6A-6D shows GPPGA Pustulation Subscore Over Time by Randomized Treatment at Day 1 and Open-Label Spesolimab Treatment at Day 8. At day 8, patients with a GPPGA total score 2 and a Generalized Pustular Psoriasis Physician Global Assessment (GPPGA) pustulation subscore 2 could receive an open-label spesolimab. GPPGA pustulation subscores from baseline are shown for: all patients randomized to spesolimab with or without receiving an open-label spesolimab dose on day 8 (FIG. 6A); patients randomized to spesolimab who did not receive an open-label spesolimab dose on day 8 (FIG. 6B); patients randomized to spesolimab who received an open-label spesolimab dose on day 8 (FIG. 6C); and placebo who received an open-label spesolimab dose on day 8 (FIG. 6D). Dashed line represents proportion of patients achieving GPPGA pustulation subscore of 0. *For this analysis, any use of escape medication, or open-label spesolimab at day 8, or rescue medication with open-label spesolimab at each timepoint were categorized separately (gray bars).

FIG. 7A-7D shows GPPGA Total Score Over Time by Randomized Treatment at Day 1 and Open-Label Spesolimab Treatment at Day 8. At day 8, patients with GPPGA total score 2 and GPPGA pustulation subscore 2 could receive an open-label spesolimab. GPPGA total scores from baseline are shown for: all patients randomized to spesolimab with or without receiving an open-label spesolimab dose on day 8 (FIG. 7A); patients randomized to spesolimab who did not receive an open-label spesolimab dose on day 8 (FIG. 7B); patients randomized to spesolimab who received an open-label spesolimab dose on day 8 (FIG. 7C); and placebo who received an open-label spesolimab dose on day 8 (FIG. 7D). Dashed line represents proportion of patients achieving GPPGA Total score of 0 or 1. *For this analysis, any use of escape medication, or open-label spesolimab at day 8, or rescue medication with open-label spesolimab at each timepoint were categorized separately (gray bars). GPP, Generalized Pustular Psoriasis; GPPGA, Generalized Pustular Psoriasis Physician Global Assessment.

FIG. 8A-8F shows the Treatment Response in Patients Who Received Up to Two Doses of Spesolimab at Day 1 and Optional Dose at Day 8 in Spesolimab Arm. FIG. 8A shows the GPPASI results; FIG. 8B shows the GPPASI 75 results; FIG. 8C shows the Pain VAS results; FIG. 8D shows the DLQI results; FIG. 8E shows the Neutrophils Over Time in Patients With Neutrophils Above the Upper Limits of Normal at Baseline (≥7×109/L); FIG. 8F shows the C-Reactive Protein Over Time in Patients Who had Levels Above the Upper Limits of Normal at Baseline (≥10 mg/L). The dataset includes observed cases in patients randomized to spesolimab who received up to two doses of spesolimab, including patients who received open-label spesolimab at day 8. The arrowhead indicates the days of intravenous spesolimab administration. For this analysis, any values post open-label spesolimab at day 8 are used, but any values post use of escape medication, or rescue medication with spesolimab are not used and imputed as the worst outcome in the calculation of median and quartiles. DLQI, Dermatology Life Quality Index; GPPASI, Generalized Pustular Psoriasis Area and Severity Index; GPPASI 75, 75% or greater improvement in the Psoriasis Area and Severity Index for Generalized Pustular Psoriasis; IQR, interquartile range; pain VAS, Pain visual analog scale.

FIG. 9A-9C shows change from baseline in GPPGA scores (including GPPGA pustulation subscore and GPPGA total score) in three patients after receiving a first and a second dose of spesolimab on days 1 and 8, respectively. FIG. 9A shows the changes in GPPGA scores (as well as skin erythema, scaling/crusting) in patient 1250001012 after receiving a first intravenous (i.v.) dose of 900 mg spesolimab and second i.v. dose of 900 mg spesolimab after 1 week. FIG. 9B shows the changes in GPPGA scores (as well as skin erythema, scaling/crusting) in patient 1276007001 after receiving a first i.v. dose of 900 mg spesolimab and second i.v. dose of 900 mg spesolimab after 1 week. FIG. 9C shows the changes in GPPGA scores (as well as skin erythema, scaling/crusting) in patient 1458001002 after receiving a first i.v. dose of 900 mg spesolimab and second i.v. dose of 900 mg spesolimab after 1 week.

FIG. 10 shows the PRO survey for PSS, Pain VAS, FACIT-Fatigue, and DLQI scores. High total scores indicate a large impairment or intense severity, except for FACIT-Fatigue, for which higher scores represent less fatigue. DLQI, Dermatology Life Quality Index; FACIT-Fatigue, Functional Assessment of Chronic Illness Therapy—Fatigue; PRO, patient-reported outcome; PSS, Psoriasis Symptom Scale; VAS, visual analogue scale.

FIG. 11A-11D shows the absolute change from baseline in PRO scores over time. FIG. 11A shows the change from baseline in Pain VAS scores, FIG. 11B shows the change from baseline in PSS scores, FIG. 11C shows the change from baseline in FACIT-Fatigue scores, FIG. 11D shows the change from baseline in DLQI scores. Efficacy results for all randomised patients using the intention-to-treat analysis showed that all four PROs continued to improve over time; however, no statistical significance versus placebo was observed during Week 1. The placebo curve begins to converge with the spesolimab curve after administration of OL spesolimab at Day 8. CI, confidence interval; DLQI, Dermatology Life Quality Index; FACIT-Fatigue, Functional Assessment of Chronic Illness Therapy—Fatigue; IV, intravenous; OL, open-label; PRO, patient-reported outcome; PSS, Psoriasis Symptom Scale; VAS, visual analogue scale.

FIG. 12 shows the distribution of maximum ADA titers in female and male patients with GPP after i.v. administration of spesolimab.

### DETAILED DESCRIPTION OF THE INVENTION

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention.

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

The inventors have surprisingly discovered inter alia that the interleukin-36 pathway inhibition with a single dose of a humanized anti-interleukin-36R (anti-IL-36R) monoclonal antibody of the present invention resulted in the rapid and sustained remission of clinical symptoms in patients with acute generalized pustular psoriasis and that no recurrence of GPP flares were observed in 20 weeks after the single dose administration.

The invention therefore relates to compositions and methods for treating and/or prophylaxis of GPP and its signs and symptoms. More specifically, the invention relates to compositions and methods for treating and/or prophylaxis of moderate to severe GPP, acute GPP, chronic GPP, and/or GPP flares in a mammal with an anti-IL36R antibody or an antigen-binding fragment thereof of the present invention. The compositions and methods include administering to the mammal a therapeutically effective amount of an anti-IL-36R antibody or an antigen-binding fragment thereof, wherein the anti-IL-36R antibody is administered in one intravenous dose. In an embodiment, the anti-IL-36R antibody is administered in one or more intravenous doses which is/are optionally followed by one or more subcutaneous doses.

Without wishing to be bound by this theory it is believed that anti-IL-36R antibodies or antigen-binding fragments thereof bind to human anti-IL-36R and thus interfere with the binding of IL-36 agonists, and in doing so block at least partially the signaling cascade from the IL-36R to inflammatory mediators. The anti-IL36R antibodies of the present invention are disclosed in U.S. Pat. No. 9,023,995 or WO2013/074569, the entire content of each of which is incorporated herein by reference.

Acute GPP flares of varying severity occur in most patients and may be idiopathic or triggered by external stimuli, such as infection, corticosteroid use or withdrawal, stress or pregnancy. Moderate or severe GPP flares cause significant morbidity and mortality due to tender, painful skin lesions, extreme fatigue, high fever, peripheral blood neutrophilia and acute phase response and sepsis. The acute phase is associated with a mean duration of hospitalization of 10 days (range 3-44 days). The observed mortality rate of 7% reported in a retrospective study with 102 GPP cases seen in a tertiary hospital in Johor, Malaysia is likely an underestimate as not all GPP patients were included in the study. Mortality rates are also likely underestimated due to lack of identifying the cause of death as GPP and are largely driven by infectious complications and extra-cutaneous organ manifestations such as renal, hepatic, respiratory and cardiac failure. After responding to treatment or spontaneous flare cessation, it is estimated that up to 50% of patients may suffer from chronic GPP characterized by persistent erythema and scaling that may also include joint symptoms.

Based on the limitations described above, current therapeutic options are not suitable for life-long treatment and do not provide sustained responses in most patients. Therefore, there is a high need to develop (i) a highly effective treatment with rapid onset of action for patients presenting with an acute GPP flare; and (ii) to develop an effective treatment of chronic GPP, which reliably prevents the occurrence of flares and is safe and tolerable for lifelong treatment.

The classic presentation of GPP flares as described by von Zumbusch is strongly correlated with polymorphisms in the IL36-R signaling pathway. Individuals with loss-of-function mutations of the IL36RN gene which encodes an endogenous IL36R antagonist (IL-36RN) have dramatically higher incidence of GPP, indicating that uncontrolled upregulation of IL36 signaling due to defective IL36RN antagonism leads to the inflammatory episodes observed in GPP. Genetic human studies have demonstrated the occurrence of GPP clusters in families with a loss of function mutation in IL36RN, which results in uncontrolled IL36R signaling. Mutations in other genes linked to the IL36 pathway such as CARD14 also lead to GPP. A recently published gene expression study indicates sustained activation of IL-1 and IL-36 in GPP, inducing neutrophil chemokine expression, infiltration, and pustule formation, suggesting that the IL-1/IL-36 inflammatory axis is a potent driver of disease pathology in GPP. Moreover, a recent meta-analysis investigated 233 published GPP cases. They found that 49 (21.0%) of 233 cases carried recessive IL36RN alleles. Those 49 recessive IL36RN alleles defined a GPP phenotype characterized by early onset and high risk of systemic inflammation.

IL36R is a cell surface receptor involved in inflammatory responses in skin and gut. It is a novel member of the IL1R family that forms a heterodimeric complex with the IL1R accessory protein. The heterodimeric IL36R system with stimulating (IL36α, IL36β, IL36γ) and inhibitory ligands (IL36Ra) shares a number of structural and functional similarities to other members of the IL1/IL1R family, such as IL1, IL18 and IL33 (R17-3602). All IL1 family members (IL1α, IL1β, IL18, IL36α, IL36β, IL36γ, and IL38) signal through a unique, cognate receptor protein which, upon ligand binding, recruits the common IL1 RacP subunit and activates NFkB and MAP kinase pathways in receptor-positive cell types. In human skin tissues, IL36R is expressed in keratinocytes, dermal fibroblasts and infiltrating myeloid cells. IL36R activation in skin tissue drives the production of inflammatory mediators (e.g. CCL20, MIP-1β, TNF-α, IL12, IL17, IL23, TGF-β) and modulates the expression of tissue remodeling genes (e.g. MMPs, TGF-β). Therefore, the link between GPP and mutations in the IL36RN is somewhat analogous to the well-established neonatal onset of sterile multifocal osteomyelitis, periostitis, and pustulosis caused by absence of interleukin-1—receptor antagonist. In this case, absence of the receptor antagonist allows unopposed action of interleukin-1, resulting in life-threatening systemic inflammation with skin and bone involvement. These clinical features responded to empirical treatment with the recombinant interleukin-1-receptor antagonist anakinra.

### I. Definitions

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile. A “powder” refers to a freeze-dried or lyophilized or a spray-dried pharmaceutical composition for parenteral use. The powder is reconstituted or dissolved typically in water. Lyophilisation is a low temperature dehydration process which involves freezing the product, lowering pressure, then removing the ice by sublimation. Freeze drying results in a high quality product because of the low temperature used in processing. For a well-developed lyophilized formulation, the shape and appearance of the product is maintained over time and the quality of the rehydrated product is excellent. Spray drying is another method of producing a dry powder from a liquid or slurry by rapidly drying with a hot gas and with the goal of achieving a consistent particle size distribution.

As used herein, the terms “intravenous dose”, “subcutaneous dose,” in addition to their ordinary meanings, refer to the temporal sequence of administration of the anti-IL-36R antibody. Thus, the “intravenous dose” is the dose which is administered at the beginning of the treatment regimen (also referred to as the “baseline dose”); it may also be referred to as an “initial dose” or “induction dose.” The “subcutaneous dose” is the dose which is administered after the intravenous dose, which may also be referred to as a “subsequent dose” or “maintenance dose.” The intravenous, subcutaneous doses may all contain the same amount of anti-IL-36R antibody or an antigen binding fragment thereof, but generally may differ from one another in terms of the amount of the antibody administered or the frequency of administration. In an embodiment, the intravenous dose is equal or larger than the subcutaneous dose. An “intravenous dose” which may be interchangeably referred to as an “initial dose” or “induction dose” can be a single dose or, alternatively, a set of doses. The subcutaneous dose which may also be referred to as a “subsequent dose” or “maintenance dose” can be a single dose or, alternatively, a set of doses for administration.

In certain embodiments, the amount of the anti-IL-36R antibody contained in the induction/initial/intravenous and maintenance/subsequent/subcutaneous doses varies from one another during the course of treatment. In certain embodiments, the one or more initial/induction/intravenous doses each comprise a first amount of the antibody or antigen-binding fragment thereof and the one or more maintenance/subsequent/subcutaneous doses each comprise a second amount of the antibody or antigen-binding fragment thereof. In some embodiments, the first amount of antibody or fragment thereof is 1.5×, 2×, 2.5×, 3×, 3.5×, 4×, or 5× the second or subsequent amount of the antibody or antigen-binding fragment thereof. In certain embodiments, one or more (e.g., 1, 2, 3, 4, or 5 or more) initial doses are administered at the beginning of the treatment regimen as “loading doses” or “leading doses” followed by subsequent doses that are administered on a less frequent basis (e.g., “maintenance doses”). In one embodiment, the intravenous dose, the induction dose or the initial dose is about 210 mg, 300 mg, 350 mg, 450 mg, 600 mg, 700 mg, 750 mg, 800 mg, 850 mg or 900 mg of the anti-IL-36R antibody. In one embodiment, the subcutaneous dose, the maintenance dose or the subsequent dose is about 150, 225 mg or 300 mg. In another embodiment, the subcutaneous dose or maintenance or subsequent dose is administered at least two weeks following the intravenous, induction or initial dose.

The terms, “antibody”, “anti-IL-36R antibody”, “humanized anti-IL-36R antibody”, “humanized anti-IL-36R epitope antibody”, and “variant humanized anti-IL-36R epitope antibody” specifically encompass monoclonal antibodies (including full length monoclonal antibodies), polyclonal antibodies, multispecific antibodies (e.g., bispecific antibodies), antibodies with minor modifications such as N- and/or C-terminal truncation, and antibody fragments such as variable domains and other portions of antibodies that exhibit a desired biological activity, e.g., IL-36R binding.

The term “monoclonal antibody” (mAb) refers to an antibody that is highly specific, being directed against a single antigenic determinant, an “epitope”. Therefore, the modifier “monoclonal” is indicative of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

The term “monomer” refers to a homogenous form of an antibody. For example, for a full-length antibody, monomer means a monomeric antibody having two identical heavy chains and two identical light chains.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized anti-IL-36R antibody composition, the result should be considered an effective treatment of the underlying disorder regardless of whether all the symptoms of the disorder are alleviated or not.

The term “prophylactically effective amount” is used to refer to an amount effective, at dosages and for periods of time necessary, to achieve the desired prophylactic result. Typically, a prophylactic dose is used in subjects prior to the onset of a GPP flare and/or prior to the onset of symptoms of GPP such as to prevent or inhibit the occurrence of acute flares. In an embodiment, a subcutaneous dose as contemplated herein is a prophylactic dose that is used in a patient with acute GPP, after the intravenous dose, to prevent a possible recurrence of the GPP flares in the patient.

### II. Antibodies

The anti-IL36R antibodies of the present invention are disclosed in U.S. Pat. No. 9,023,995 or WO2013/074569, the entire content of each of which is incorporated herein by reference.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

Mode of Action

An anti-IL-36R antibody of the present invention is a humanized antagonistic monoclonal IgG1 antibody that blocks human IL36R signaling. Binding of an anti-IL-36R antibody of the present invention to IL36R is anticipated to prevent the subsequent activation of IL36R by cognate ligands (IL36 α, β and γ) and downstream activation of pro-inflammatory and pro-fibrotic pathways with the aim to reduce epithelial cell/fibroblast/immune cell-mediated inflammation and interrupt the inflammatory response that drives pathogenic cytokine production in generalized pustular psoriasis (GPP). As provided herein, an anti-IL-36R antibody of the present invention has been tested and proved to be effective in treating patients with acute Generalized Pustular Psoriasis (GPP), a severe inflammatory skin disease driven by uncontrolled IL36 activity.

IL-36R is also known as IL-1 RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1RAcP). IL-36 antagonist ligands (IL-36RA/IL1F5, IL-38/ILF10) inhibit the signaling cascade.

Variable regions and CDRs of representative antibodies of the present invention are disclosed below:

### Anti-IL-36R Mouse Antibody Sequences

Variable regions and CDRs of representative mouse lead antibodies of the present invention (mouse leads) are shown below:

### Light Chain Variable Region (VK) Amino Acid Sequences

>33D10612vK Protein (antibody 33D10)
QIVLTQSPAIMSASLGERVTMTCTASSSVSSSYLHWYQKKPGSSPKLWVYSTSNLASGVPVRF
SGSGSGTSYSLTISSMEAEDAATYYCHQHHRSPVTFGSGTKLEMK (SEQ ID NO: 1)
>172C8B12 vK protein (antibody 172C8)
DIQMTQSPASQSASLGESVTFTCLASQTIGTWLAWYQQRPGKSPQLLIYAATSLADGVPSRFS
GSGSGTQFSFNIRSLQAEDFASYYCQQVYTTPLTFGGGTKLEIK (SEQ ID NO: 2)
>67E7E8 vK protein (antibody 67E7)
DIQMTQSPASQSASLGESVTFTCLASQTIGTWLGWYQQKPGKSPQLLIYRSTTLADGVPSRFS
GSGSGTKFSFKISSLQAADFASYYCQQLYSAPYTFGGGTKLEIR (SEQ ID NO: 3)
>78C8D1 vK Protein (antibody 78C8)
DVLLTQTPLSLPVSLGDQASISCRSSQNIVHSNGNTYLQWYLQKPGQSPKLLIYKVSNRFSGVP
DRFSGSGSGTDFTLKISRVEAEDLGVYYCFQGSHVPFTFGAGTKLELK (SEQ ID NO: 4)
>81A1D1 vK Protein (antibody 81A1)
DIQMTQTTSSLSASLGDRVTISCRASQDIYKYLNWYQQKPDGTLKLLIYYTSGLHSGVPSRFSG
SGSGTDFSLTISNLEPEDIATYFCQQDSKFPWTFGGDTKLEIK (SEQ ID NO: 5)
>81B4E11 vK Protein (antibody 81B4)
QIVLTQSPAIMSASLGERVTMTCTASSSVSSSYFHWYQQKPGSSPKLWIYRTSNLASGVPGRF
SGSGSGTSYSLTISSMEAEDAATYYCHQFHRSPLTFGAGTKLELK (SEQ ID NO: 6)
>73C5C10 vK protein (antibody 73C5)
DIVMTQSQKFLSTSVGVRVSVTCKASQDVGTNVLWYQQKIGQSPKPLIYSASYRHSGVPDRFT
GSGSGTDFTLIISNVQSEDLAEYFCQQYSRYPLTFGPGTKLELK (SEQ ID NO: 7)
>73F6F8 vK protein (antibody 73F6)
DIVMTQSQKFLSTSVGVRVSVTCKASQDVGTNVLWYQQKIGQSPKALIYSASYRHSGVPDRFT
GSGSGTDFTLIITNVQSEDLAEYFCQQYSRYPLTFGPGTKLELK (SEQ ID NO: 8)
>76E10E8 vK protein (antibody 76E10)
DIVMTQSQKFMSATVGGRVNITCKASQNVGRAVAWYQQKPGQSPKLLTHSASNRYTGVPDRF
TGSGSGTDFTLTITNMQSEDLADYFCQQYSSYPLTFGAGTKLDLK (SEQ ID NO: 9)
>89A12B8 vK protein (antibody 89A12)
DIQMTQSPASQSASLGESVTFSCLASQTIGTWLGWYQQKPGKSPQLLIYRATSLADGVPSRFS
GSGSGTNFSFKISSLQAEDLASYYCQQLYSGPYTFGGGTKLEIR (SEQ ID NO: 10)

### Heavy Chain Variable Region (VH) Amino Acid Sequences

>33D10B12vH Protein (antibody 33D10)
QVQLQQSGTELLKPGASVKLSCKASGNTVTSYWMHWVKQRPGQGLEWIGEILPSTGRTNYNE
NFKGKAMLTVDKSSSTAYMQLSSLASEDSAVYYCTIVYFGNPWFAYWGQGTLVTVSA (SEQ
ID NO: 11)
>172C8B12 vH protein (antibody 172C8)
EVQLQQSGPELVKPGASVKLSCKASGYTFTDNYMNWVRQSHGKSLEWIGRVNPSNGDTKYN
QNFKGKATLTVDKSLSTAYMQLNGLTSEDSAVYYCGRTKNFYSSYSYDDAMDYWGQGTSVTV
SS (SEQ ID NO: 12)
>67E7E8 vH protein (antibody 67E7)
EVQLQQSGAEFVRPGASVKFSCTASGFNIKDDYIHWVRQRPEQGLEWVGRIDPANGNTKYAP
KFQDKATITADTSSNTAYLQLSSLTSEDTAVYYCAKSFPNNYYSYDDAFAYWGQGTLVTVSA
(SEQ ID NO: 13)
>78C8D1 vH Protein (antibody 78C8)
QVQLKESGPVLVAPSQSLSITCTVSGFSLTKFGVHWIRQTPGKGLEWLGVIWAGGPTNYNSAL
MSRLTISKDISQSQVFLRIDSLQTDDTAMYYCAKQIYYSTLVDYWGQGTSVTVSS (SEQ ID NO:
14)
>81A1D1 vH Protein (antibody 81A1)
QVQLKESGPGLVAPSQSLFITCTVSGFSLSSYEINWVRQVPGKGLEWLGVIWTGITTNYNSALI
SRLSISKDNSKSLVFLKMNSLQTDDTAIYYCARGTGTGFYYAMDYWGQGTSVTVSS (SEQ ID
NO: 15)
>81B4E11 vH Protein (antibody 81B4)
QVQLQQPGADFVRPGASMRLSCKASGYSFTSSWIHWVKQRPGQGLEWIGEINPGNVRTNYN
ENFRNKATLTVDKSSTTAYMQLRSLTSADSAVYYCTVVFYGEPYFPYWGQGTLVTVSA (SEQ
ID NO: 16)
>73C5C10 vH Protein (antibody 73C5)
QVQLKESGPGLVAPSQSLSITCTVSGFSLTNYAVHWVRQFPGKGLEWLGVIWSDGSTDFNAP
FKSRLSINKDNSKSQVFFKMNSLQIDDTAIYYCARKGGYSGSWFAYWGQGTLVTVSA (SEQ ID
NO: 17)
>73F6F8 vH protein (antibody 73F6)
QVQLKESGPGLVAPSQSLSITCTVSGFSLTNYAVHWVRQFPGKGLEWLGVIWSDGSTDYNAP
FKSRLSINKDNSKSQVFFKMNSLQTDDTAIYYCARKGGYSGSWFAYWGQGTLVTVSA (SEQ
ID NO: 18)
>76E10E8 vH protein (antibody 76E10)
QVQLKESGPVLVAPSQSLSITCTVSGFSLTNYGVHWVRQPPGKGLEWLGVIWPVGSTNYNSA
LMSRLSIHKDNSKSQVFLRMNSLQTDDTAIYYCAKMDWDDFFDYWGQGTTLTVSS(SEQ ID
NO: 19)
>89A12B8 vH Protein (antibody 89A12)
EVQLQQSGAELVRPGASVRLSCTASGFNIKDDYIHWVRQRPKQGLEWLGRIDPANGNTKYDP
RFQDKATITADTSSNTAYLHLSSLTSEDTAVYYCAKSFPDNYYSYDDAFAYWGQGTLVTVSA
(SEQ ID NO: 20)

### Light Chain CDR-1 (L-CDR1) Amino Acid Sequences

>33D10G1 L-CDR1
(SEQ ID NO: 21)
TASSSVSSSYLH
>172C8B12 L-CDR1
(SEQ ID NO: 22)
LASQTIGTWLA
>67E7E8 L-CDR1
(SEQ ID NO: 23)
LASQTIGTWLG
>78C8D1 L-CDR1
(SEQ ID NO: 24)
RSSQNIVHSNGNTYLQ
>81A1D1 L-CDR1
(SEQ ID NO: 25)
RASQDIYKYLN
>81B4E11 L-CDR1
(SEQ ID NO: 26)
TASSSVSSSYFH
>73C5C10 L-CDR1
(SEQ ID NO: 27)
KASQDVGTNVL
>73F6F8 L-CDR1
(SEQ ID NO: 27)
KASQDVGTNVL
>76E10E8 L-CDR1
(SEQ ID NO: 28)
KASQNVGRAVA
>89Al2B8 L-CDR1
(SEQ ID NO: 29)
LASQTIGTWLG

### Light Chain CDR-2 (L-CDR2) Amino Acid Sequences

>33D10B12 L-CDR2
(SEQ ID NO: 30)
STSNLAS
>172C8B12 L-CDR2
(SEQ ID NO: 31)
AATSLAD
>67E7E8 L-CDR2
(SEQ ID NO: 32)
RSTTLAD
>78C8D1 L-CDR2
(SEQ ID NO: 33)
KVSNRFS
>81A1D1 L-CDR2
(SEQ ID NO: 34)
YTSGLHS
>81B4E11 L-CDR2
(SEQ ID NO: 35)
RTSNLAS
>73C5C10 L-CDR2
(SEQ ID NO: 36)
SASYRHS
>73F6F8 L-CDR2
(SEQ ID NO: 36)
SASYRHS
>76E10E8 L-CDR2
(SEQ ID NO: 37)
SASNRYT
>89Al2B8 L-CDR2
(SEQ ID NO: 38)
RATSLAD

### Light Chain CDR-3 (L-CDR3) Amino Acid Sequences

>33D10B12 L-CDR3
(SEQ ID NO: 39)
HQHHRSPVT
>172C8B12 L-CDR3
(SEQ ID NO: 40)
QQVYTTPLT
>67E7E8 L-CDR3
(SEQ ID NO: 41)
QQLYSAPYT
>78C8D1 L-CDR3
(SEQ ID NO: 42)
FQGSHVPFT
>81A1D1 L-CDR3
(SEQ ID NO: 43)
QQDSKFPWT
>81B4E11 L-CDR3
(SEQ ID NO: 44)
HQFHRSPLT
>73C5C10 L-CDR3
(SEQ ID NO: 45)
QQYSRYPLT
>73F6F8 L-CDR3
(SEQ ID NO: 45)
QQYSRYPLT
>76E10E8 L-CDR3
(SEQ ID NO: 46)
QQYSSYPLT
>89Al2B8 L-CDR3
(SEQ ID NO: 47)
QQLYSGPYT

### Heavy Chain CDR-1 (H-CDR1) Amino Acid Sequences

>33D10B12 H-CDR1
(SEQ ID NO: 48)
GNTVTSYWMH
>172C8B12 H-CDR1
(SEQ ID NO: 49)
GYTFTDNYMN
>67E7E8 H-CDR1
(SEQ ID NO: 50)
GFNIKDDYIH
>78C8D1 H-CDR1
(SEQ ID NO: 51)
GFSLTKFGVH
>81A1D1 H-CDR1
(SEQ ID NO: 52)
GFSLSSYEIN
>81B4E11 H-CDR1
(SEQ ID NO: 53)
GYSFTSSWIH
>73C5C10 H-CDR1
(SEQ ID NO: 54)
GFSLTNYAVH
>73F6F8 H-CDR1
(SEQ ID NO: 54)
GFSLTNYAVH
>76E10E8 H-CDR1
(SEQ ID NO: 55)
GFSLTNYGVH
>89Al2B8 H-CDR1
(SEQ ID NO: 56)
GFNIKDDYIH

### Heavy Chain CDR-2 (H-CDR2) Amino Acid Sequences

>33D10B12 H-CDR2
(SEQ ID NO: 57)
EILPSTGRTNYNENFKG
>172C8B12 H-CDR2
(SEQ ID NO: 58)
RVNPSNGDTKYNQNFKG
>67E7E8 H-CDR2
(SEQ ID NO: 59)
RIDPANGNTKYAPKFQD
>78C8D1 H-CDR2
(SEQ ID NO: 60)
VIWAGGPTNYNSALMS
>81A1D1 H-CDR2
(SEQ ID NO: 61)
VIWTGITTNYNSALIS
>81B4E11 H-CDR2
(SEQ ID NO: 62)
EINPGNVRTNYNENF
>73C5C10 H-CDR2
(SEQ ID NO: 63)
VIWSDGSTDFNAPFKS
>73F6F8 H-CDR2
(SEQ ID NO: 64)
VIWSDGSTDYNAPFKS
>76E10E8 H-CDR2
(SEQ ID NO: 65)
VIWPVGSTNYNSALMS
>89Al2B8 H-CDR2
(SEQ ID NO: 66)
RIDPANGNTKYDPRFQD

### Heavy Chain CDR-3 (H-CDR3) Amino Acid Sequences

>33D10B12 H-CDR3
(SEQ ID NO: 67)
VYFGNPWFAY
>172C8B12 H-CDR3
(SEQ ID NO: 68)
TKNFYSSYSYDDAMDY
>67E7E8 H-CDR3
(SEQ ID NO: 69)
SFPNNYYSYDDAFAY
>78C8D1 H-CDR3
(SEQ ID NO: 70)
QIYYSTLVDY
>81A1D1 H-CDR3
(SEQ ID NO: 71)
GTGTGFYYAMDY
>81B4E11 H-CDR3
(SEQ ID NO: 72)
VFYGEPYFPY
>73C5C10 H-CDR3
(SEQ ID NO: 73)
KGGYSGSWFAY
>73F6F8 H-CDR3
(SEQ ID NO: 73)
KGGYSGSWFAY
>76E10E8 H-CDR3
(SEQ ID NO: 74)
MDWDDFFDY
>89Al2B8 H-CDR3
(SEQ ID NO: 75)
SFPDNYYSYDDAFAY

### Anti-IL-36R Mouse CDR Sequences

A summary of the CDR sequences of the lead mouse antibodies is shown below:

| Antibody | H-CDR Sequences | L-CDR Sequences |
|---|---|---|
| 33D10 | GNTVTSYWMH (H-CDR1) | TASSSVSSSYLH (L- |
| | SEQ ID No: 48 | CDR1) SEQ ID No: 21 |
| | EILPSTGRTNYNENFKG | STSNLAS (L-CDR2) SEQ |
| | (H-CDR2) SEQ ID No: 57 | ID No: 30 |
| | VYFGNPWFAY (H-CDR3) | HQHHRSPVT (L-CDR3) |
| | SEQ ID No: 67 | SEQ ID No: 39 |
| 172C8 | GYTFTDNYMN (H-CDR1) | LASQTIGTWLA (L-CDR1) |
| | SEQ ID No: 49 | SEQ ID No: 22 |
| | RVNPSNGDTKYNQNFKG | AATSLAD (L-CDR2) SEQ |
| | (H-CDR2) SEQ ID No: 58 | ID No: 31 |
| | TKNFYSSYSYDDAMDY | QQVYTTPLT (L-CDR3) |
| | (H-CDR3) SEQ ID No: 68 | SEQ ID No: 40 |
| 67E7 | GFNIKDDYIH (H-CDR1) | LASQTIGTWLG (L-CDR1) |
| | SEQ ID No: 50 | SEQ ID No: 23 |
| | RIDPANGNTKYAPKFQD | RSTTLAD (L-CDR2) SEQ |
| | (H-CDR2) SEQ ID No: 59 | ID No: 32 |
| | SFPNNYYSYDDAFAY (H- | QQLYSAPYT (L-CDR3) |
| | CDR3) SEQ ID No: 69 | SEQ ID No: 41 |
| 78C8 | GFSLTKFGVH (H-CDR1) | RSSQNIVHSNGNTYLQ (L- |
| | SEQ ID No: 51 | CDR1) SEQ ID No: 24 |
| | VIWAGGPTNYNSALMS | KVSNRFS (L-CDR2) SEQ |
| | (H-CDR2) SEQ ID No: 60 | ID No: 33 |
| | QIYYSTLVDY (H-CDR3) | FQGSHVPFT (L-CDR3) |
| | SEQ ID No: 70 | SEQ ID No: 42 |
| 81A1 | GFSLSSYEIN (H-CDR1) | RASQDIYKYLN (L-CDR1) |
| | SEQ ID No: 52 | SEQ ID No: 25 |
| | VIWTGITTNYNSALIS (H- | YTSGLHS (L-CDR2) SEQ |
| | CDR2) SEQ ID No: 61 | ID No: 34 |
| | GTGTGFYYAMDY (H- | QQDSKFPWT (L-CDR3) |
| | CDR3) SEQ ID No: 71 | SEQ ID No: 43 |
| 81B4 | GYSFTSSWIH (H-CDR1) | TASSSVSSSYFH (L- |
| | SEQ ID No: 53 | CDR1) SEQ ID No: 26 |
| | EINPGNVRTNYNENF (H- | RTSNLAS (L-CDR2) SEQ |
| | CDR2) SEQ ID No: 62 | ID No: 35 |
| | VFYGEPYFPY (H-CDR3) | HQFHRSPLT (L-CDR3) |
| | SEQ ID No: 72 | SEQ ID No: 44 |
| 73C5 | GFSLTNYAVH (H-CDR1) | KASQDVGTNVL (L-CDR1) |
| | SEQ ID No: 54 | SEQ ID No: 27 |
| | VIWSDGSTDFNAPFKS (H- | SASYRHS (L-CDR2) SEQ |
| | CDR2) SEQ ID No: 63 | ID No: 36 |
| | KGGYSGSWFAY (H- | QQYSRYPLT (L-CDR3) |
| | CDR3) SEQ ID No: 73 | SEQ ID No: 45 |
| 73F6 | GFSLTNYAVH (H-CDR1) | KASQDVGTNVL (L-CDR1) |
| | SEQ ID No: 54 | SEQ ID No: 27 |
| | VIWSDGSTDYNAPFKS | SASYRHS (L-CDR2) SEQ |
| | (H-CDR2) SEQ ID No: 64 | ID No: 36 |
| | KGGYSGSWFAY (H- | QQYSRYPLT (L-CDR3) |
| | CDR3) SEQ ID No: 73 | SEQ ID No: 45 |
| 76E10 | GFSLTNYGVH (H-CDR1) | KASQNVGRAVA (L-CDR1) |
| | SEQ ID No: 55 | SEQ ID No: 28 |
| | VIWPVGSTNYNSALMS | SASNRYT (L-CDR2) SEQ |
| | (H-CDR2) SEQ ID No: 65 | ID No: 37 |
| | MDWDDFFDY (H-CDR3) | QQYSSYPLT (L-CDR3) |
| | SEQ ID No: 74 | SEQ ID No: 46 |
| 89A12 | GFNIKDDYIH (H-CDR1) | LASQTIGTWLG (L-CDR1) |
| | SEQ ID No: 56 | SEQ ID No: 29 |
| | RIDPANGNTKYDPRFQD | RATSLAD (L-CDR2) SEQ |
| | (H-CDR2) SEQ ID No: 66 | ID No: 38 |
| | SFPDNYYSYDDAFAY (H- | QQLYSGPYT (L-CDR3) |
| | CDR3) SEQ ID No: 75 | SEQ ID No: 47 |

### Anti-IL-36R Humanized Antibody Sequences

Human framework sequences were selected for the mouse leads based on the framework homology, CDR structure, conserved canonical residues, conserved interface packing residues and other parameters to produce humanized variable regions (see Example 5).

Representative humanized variable regions derived from antibodies 81B4 and 73C5 are shown below.

### Light Chain Variable Region (VK) Amino Acid Sequences

>81B4vK32_3 vK protein
(SEQ ID NO: 76)
EIVLTQSPGTLSLSPGERATMSCTASSSVSSSYFHWYQQKPGQAPRLLIY
RTSTLASGIPDRFSGSGSGTDFTLTISRLEPEDAATYYCHQFHRSPLTFG
QGTKLEIK
>81B4vK32_105 vK protein
(SEQ ID NO: 77)
EIVLTQSPGTLSLSPGERATMSCTASSSVSSSYFHWYQQKPGQAPRLLIY
RTSILASGVPDRFSGSGSGTDFTLTISRLEPEDFATYYCHQFHRSPLTFG
QGTKLEIK
>81B4vK32_116 vK protein
(SEQ ID NO: 78)
EIVLTQSPGTLSLSPGERATMSCTASSSVSSSYFHWYQQKPGQAPRLWIY
RTSRLASGVPDRFSGSGSGTDFTLTISRLEPEDAATYYCHQFHRSPLTFG
QGTKLEIK
>81B4vK32_127 vK protein
(SEQ ID NO: 79)
EIVLTQSPGTLSLSPGERATMTCTASSSVSSSYFHWYQQKPGQAPRLLIY
RTSRLASGVPDRFSGSGSGTDFTLTISRLEPEDFAVYYCHQFHRSPLTFG
QGTKLEIK
>81B4vK32_138 vK protein
(SEQ ID NO: 80)
QIVLTQSPGTLSLSPGERATMTCTASSSVSSSYFHWYQQKPGQAPRLWIY
RTSRLASGVPDRFSGSGSGTDFTLTISRLEPEDAATYYCHQFHRSPLTFG
AGTKLEIK
>81B4vK32_140 vK protein
(SEQ ID NO: 81)
QIVLTQSPGTLSLSPGERVTMSCTASSSVSSSYFHWYQQKPGQAPRLLIY
RTSQLASGIPDRFSGSGSGTDFTLTISRLEPEDAATYYCHQFHRSPLTFG
QGTKLEIK
>81B4vK32_141 vK protein
(SEQ ID NO: 82)
QIVLTQSPGTLSLSPGERATMTCTASSSVSSSYFHWYQQKPGQAPRLLIY
RTSKLASGVPDRFSGSGSGTDFTLTISRLEPEDFATYYCHQFHRSPLTFG
QGTKLEIK
>81B4vK32 147 vK protein
(SEQ ID NO: 83)
EIVLTQSPGTLSLSPGERATMSCTASSSVSSSYFHWYQQKPGQAPRLLIY
RTSHLASGIPGRFSGSGSGTDFTLTISRLEPEDAAVYYCHQFHRSPLTFG
QGTKLEIK
>73C5vK39_2 vK protein
(SEQ ID NO: 84)
EIVMTQSPATLSVSPGVRATLSCKASQDVGTNVLWYQQKPGQAPRPLIYS
ASYRHSGIPDRFSGSGSGTEFTLTISSLQSEDFAEYFCQQYSRYPLTFGQ
GTKLEIK
>73C5vK39_7 vK protein
(SEQ ID NO: 85)
EIVMTQSPATLSVSPGVRATLSCKASQDVGTNVLWYQQKPGQAPRPLIYS
ASYRHSGIPDRFSGSGSGTEFTLTISSLQSEDFAVYYCQQYSRYPLTFGQ
GTKLEIK
>73C5vK39_15 vK protein
(SEQ ID NO: 86)
EIVMTQSPATLSVSPGVRATLSCKASQDVGTNVLWYQQKPGQAPRPLIYS
ASYRHSGIPARFSGSGSGTEFTLTISSLQSEDFAEYYCQQYSRYPLTFGQ
GTKLEIK

### Heavy Chain Variable Region (VH) Amino Acid Sequences

>81B4vH33_49 vH Protein
(SEQ ID NO: 87)
QVQLVQSGAEVKKPGASVKVSCKASGYSFTSSWIHWVRQAPGQGLEWIGE
INPGNVRTNYNENFRNKATMTVDTSISTAYMELSRLRSDDTAVYYCAVVF
YGEPYFPYWGQGTLVTVSS
>81B4vH33_85T vH Protein
(SEQ ID NO: 88)
QVQLVQSGAEVKKPGASVKVSCKASGYSFTSSWIHWVRQRPGQGLEWIGE
INPGNVRTNYNENFRNRVTMTVDTSISTAYMELSRLRSDDTAVYYCTVVF
YGEPYFPYWGQGTLVTVSS
>81B4vH33_90 vH Protein
(SEQ ID NO: 89)
QVQLVQSGAEVKKPGASVKVSCKASGYSFTSSWIHWVKQAPGQGLEWMGE
INPGNVRTNYNENFRNKVTMTVDTSISTAYMELSRLRSDDTAVYYCTVVF
YGEPYFPYWGQGTLVTVSS
>81B4vH33_93 vH Protein
(SEQ ID NO: 90)
QVQLVQSGAEVKKPGASVKVSCKASGYSFTSSWIHWVRQRPGQGLEWMGE
INPGNVRTNYNENFRNRATLTRDTSISTAYMELSRLRSDDTAVYYCAVVF
YGEPYFPYWGQGTLVTVSS
>81B4vH50_22 vH Protein
(SEQ ID NO: 91)
QVQLVQSGAEVKKPGASVKVSCKASGYSFTSSWIHWVRQRPGQGLEWMGE
ILPGVVRTNYNENFRNKVTMTVDTSISTAYMELSRLRSDDTAVYYCTVVF
YGEPYFPYWGQGTLVTVSS
>81B4vH50_30 vH Protein
(SEQ ID NO: 92)
QVQLVQSGAEVKKPGASVKVSCKASGYSFTSSWIHWVRQRPGQGLEWIGE
INPGAVRTNYNENFRNRVTMTVDTSISTAYMELSRLRSDDTAVYYCTVVF
YGEPYFPYWGQGTLVTVSS
>81B4vH51_13 vH Protein
(SEQ ID NO: 93)
QVQLVQSGAEVKKPGASVKVSCKASGYSFTSSWIHWVRQAPGQGLEWIGE
INPGLVRTNYNENFRNKVTMTVDTSISTAYMELSRLRSDDTAVYYCAVVF
YGEPYFPYWGQGTLVTVSS
>81B4vH51_15 vH Protein
(SEQ ID NO: 94)
QVQLVQSGAEVKKPGASVKVSCKASGYSFTSSWIHWVRQAPGQGLEWIGE
INPGAVRTNYNENFRNKVTMTVDTSISTAYMELSRLRSDDTAVYYCAVVF
YGEPYFPYWGQGTLVTVSS
>81B4vH52_83 vH Protein
(SEQ ID NO: 95)
QVQLVQSGAEVKKPGASVKVSCKASGYSFTSSWIHWVRQAPGQGLEWIGE
INPGSVRTNYNENFRNKATMTVDTSISTAYMELSRLRSDDTAVYYCAVVF
YGEPYFPYWGQGTLVTVSS
>73C5vH46_4 vH Protein
(SEQ ID NO: 96)
QVQLQESGPGLVKPSETLSITCTVSGFSLTDYAVHWIRQPPGKGLEWIGV
IWSDGSTDYNAPFKSRVTINKDTSKSQVSFKMSSVQAADTAVYYCARKGG
YSGSWFAYWGQGTLVTVSS
>73C5vH46_19 vH Protein
(SEQ ID NO: 97)
QVQLQESGPGLVKPSETLSITCTVSGFSLTDYAVHWIRQPPGKGLEWIGV
IWSDGSTDYNAPFKSRVTISKDTSKNQVSLKMNSLTTDDTAVYYCARKGG
YSGSWFAYWGQGTLVTVSS
>73C5vH46_40 vH Protein
(SEQ ID NO: 98)
QVQLQESGPGLVKPSETLSITCTVSGFSLTDYAVHWIRQPPGKGLEWIGV
IWSDGSTDYNAPFKSRVTISKDNSKSQVSLKMNSVTVADTAVYYCARKGG
YSGSWFAYWGQGTLVTVSS
>73C5vH47_65 vH Protein
(SEQ ID NO: 99)
QVQLQESGPGLVKPSETLSITCTVSGFSLTDYAVHWVRQPPGKGLEWIGV
IWSDGSTDYNAPFKSRVTISKDTSKNQVSFKLSSVTVDDTAVYYCARKGG
YSGSWFAYWGQGTLVTVSS
>73C5vH47_77 vH Protein
(SEQ ID NO: 100)
QVQLQESGPGLVAPSETLSLTCTVSGFSLTDYAVHWIRQFPGKGLEWIGV
IWSDGSTDFNAPFKSRVTISKDTSKNQVSFKLSSVTTDDTAVYYCARKGG
YSGSWFAYWGQGTLVTVSS
>73C5vH58_91 vH Protein
(SEQ ID NO: 101)
QVQLQESGPGLVKPSETLSITCTVSGFSLTDYAVHWIRQPPGKGLEWIGV
IWSDGSTDYNAPFKSRVTISKDNSKSQVSFKMSSVTADDTAVYYCARKGG
YSGSWFAYWGQGTLVTVSS

The CDR sequences from the humanized variable regions derived from antibodies 81B4 and 73C5 shown above are depicted below.

### L-CDR1 Amino Acid Sequences

>81B4vK32_3 L-CDR1
(SEQ ID NO: 26)
TASSSVSSSYFH
>81B4vK32_105 L-CDR1
(SEQ ID NO: 26)
TASSSVSSSYFH
>81B4vK32_116 L-CDR1
(SEQ ID NO: 26)
TASSSVSSSYFH
>81B4vK32_127 L-CDR1
(SEQ ID NO: 26)
TASSSVSSSYFH
>81B4vK32_138 L-CDR1
(SEQ ID NO: 26)
TASSSVSSSYFH
>81B4vK32_140 L-CDR1
(SEQ ID NO: 26)
TASSSVSSSYFH
>81B4vK32_141 L-CD R1
(SEQ ID NO: 26)
TASSSVSSSYFH
>81B4vK32_147 L-CDR1
(SEQ ID NO: 26)
TASSSVSSSYFH
>73C5vK39_2 L-CDR1
(SEQ ID NO: 27)
KASQDVGTNVL
>73C5vK39_7 L-CDR1
(SEQ ID NO: 27)
KASQDVGTNVL
>73C5vK39_15 L-CDR1
(SEQ ID NO: 27)
KASQDVGTNVL

### L-CDR2 Amino Acid Sequences

>81B4vK32_3 L-CDR2
(SEQ ID 102)
RTSTLAS
>81B4vK32_105 L-CDR2
(SEQ ID 103)
RTSILAS
>81B4vK32_116 L-CDR2
(SEQ ID 104)
RTSRLAS
>81B4vK32_127 L-CDR2
(SEQ ID 104)
RTSRLAS
>81B4vK32_138 L-CDR2
(SEQ ID 104)
RTSRLAS
>81B4vK32_140 L-CDR2
(SEQ ID 105)
RTSQLAS
>81B4vK32_141 L-CDR2
(SEQ ID 106)
RTSKLAS
>81B4vK32_147 L-CDR2
(SEQ ID 140)
RTSHLAS
>73C5vK39_2 L-CDR2
(SEQ ID NO: 36)
SASYRHS
>73C5vK39_7 L-CDR2
(SEQ ID NO: 36)
SASYRHS
>73C5vK39_15 L-CDR2
(SEQ ID NO: 36)
SASYRHS

### L-CDR3 Amino Acid Sequences

>81B4vK32_3 L-CDR3
(SEQ ID NO: 44)
HQFHRSPLT
>81B4vK32_105 L-CDR3
(SEQ ID NO: 44)
HQFHRSPLT
>81B4vK32_116 L-CDR3
(SEQ ID NO: 44)
HQFHRSPLT
>81B4vK32_127 L-CDR3
(SEQ ID NO: 44)
HQFHRSPLT
>81B4vK32_138 L-CDR3
(SEQ ID NO: 44)
HQFHRSPLT
>81B4vK32_140 L-CDR3
(SEQ ID NO: 44)
HQFHRSPLT
>81B4vK32_141 L-CDR3
(SEQ ID NO: 44)
HQFHRSPLT
>81B4vK32_147 L-CDR3
(SEQ ID NO: 44)
HQFHRSPLT
>73C5vK39_2 L-CDR3
(SEQ ID NO: 45)
QQYSRYPLT
>73C5vK39_7 L-CDR3
(SEQ ID NO: 45)
QQYSRYPLT
>73C5vK39_15 L-CDR3
(SEQ ID NO: 45)
QQYSRYPLT

### H-CDR1 Amino Acid Sequences

>81B4vH33_49 H-CDR1
(SEQ ID NO: 53)
GYSFTSSWIH
>81B4vH33_85T H-CDR1
(SEQ ID NO: 53)
GYSFTSSWIH
>81B4vH33_90 H-CDR1
(SEQ ID NO: 53)
GYSFTSSWIH
>81B4vH33_93 H-CDR1
(SEQ ID NO: 53)
GYSFTSSWIH
>81B4vH50_22 H-CDR1
(SEQ ID NO: 53)
GYSFTSSWIH
>81B4vH50_30 H-CDR1
(SEQ ID NO: 53)
GYSFTSSWIH
>81B4vH51_13 H-CDR1
(SEQ ID NO: 53)
GYSFTSSWIH
>81B4vH51_15 H-CDR1
(SEQ ID NO: 53)
GYSFTSSWIH
>81B4vH52_83 H-CDR1
(SEQ ID NO: 53)
GYSFTSSWIH
>73C5vH46_4 H-CDR1
(SEQ ID NO: 107)
GFSLTDYAVH
>73C5vH46_19 H-CDR1
(SEQ ID NO: 107)
GFSLTDYAVH
>73C5vH46_40 H-CDR1
(SEQ ID NO: 107)
GFSLTDYAVH
>73C5vH47_65 H-CDR1
(SEQ ID NO: 107)
GFSLTDYAVH
>73C5vH47_77 H-CDR1
(SEQ ID NO: 107)
GFSLTDYAVH
>73C5vH58_91 H-CDR1
(SEQ ID NO: 107)
GFSLTDYAVH

### H-CDR2 Amino Acid Sequences

>81B4vH33_49 H-CDR2
(SEQ ID NO: 62)
EINPGNVRTNYNENF
>81B4vH33_85T H-CDR2
(SEQ ID NO: 62)
EINPGNVRTNYNENF
>81B4vH33_90 H-CDR2
(SEQ ID NO: 62)
EINPGNVRTNYNENF
>81B4vH33_93 H-CDR2
(SEQ ID NO: 62)
EINPGNVRTNYNENF
>81B4vH50_22 H-CDR2
(SEQ ID NO: 108)
EILPGVVRTNYNENF
>81B4vH50_30 H-CDR2
(SEQ ID NO: 109)
EINPGAVRTNYNENF
>81B4vH51_13 H-CDR2
(SEQ ID NO: 110)
EINPGLVRTNYNENF
>81B4vH51_15 H-CDR2
(SEQ ID NO: 109)
EINPGAVRTNYNENF
>81B4vH52_83 H-CDR2
(SEQ ID NO: 111)
EINPGSVRTNYNENF
>73C5vH46_4 H-CDR2
(SEQ ID NO: 64)
VIWSDGSTDYNAPFKS
>73C5vH46_19 H-CDR2
(SEQ ID NO: 64)
VIWSDGSTDYNAPFKS
>73C5vH46_40 H-CDR2
(SEQ ID NO: 64)
VIWSDGSTDYNAPFKS
>73C5vH47_65 H-CDR2
(SEQ ID NO: 64)
VIWSDGSTDYNAPFKS
>73C5vH47_77 H-CDR2
(SEQ ID NO: 63)
VIWSDGSTDFNAPFKS
>73C5vH58_91 H-CDR2
(SEQ ID NO: 64)
VIWSDGSTDYNAPFKS

### H-CDR3 Amino Acid Sequences

>81B4vH33_49 H-CDR3
(SEQ ID NO: 72)
VFYGEPYFPY
>81B4vH33_85T H-CDR3
(SEQ ID NO: 72)
VFYGEPYFPY
>81B4vH33_90 H-CDR3
(SEQ ID NO: 72)
VFYGEPYFPY
>81B4vH33_93 H-CDR3
(SEQ ID NO: 72)
VFYGEPYFPY
>81B4vH50_22 H-CDR3
(SEQ ID NO: 72)
VFYGEPYFPY
>81B4vH50_30 H-CDR3
(SEQ ID NO: 72)
VFYGEPYFPY
>81B4vH51_13 H-CDR3
(SEQ ID NO: 72)
VFYGEPYFPY
>81B4vH51_15 H-CDR3
(SEQ ID NO: 72)
VFYGEPYFPY
>81B4vH52_83 H-CDR3
(SEQ ID NO: 72)
VFYGEPYFPY
>73C5vH46_4 H-CDR3
(SEQ ID NO: 73)
KGGYSGSWFAY
>73C5vH46_19 H-CDR3
(SEQ ID NO: 73)
KGGYSGSWFAY
>73C5vH46_40 H-CDR3
(SEQ ID NO: 73)
KGGYSGSWFAY
>73C5vH47_65 H-CDR3
(SEQ ID NO: 73)
KGGYSGSWFAY
>73C5vH47_77 H-CDR3
(SEQ ID NO: 73)
KGGYSGSWFAY
>73C5vH58_91 H-CDR3
(SEQ ID NO: 73)
KGGYSGSWFAY

In one aspect, a variable region of the present invention is linked to a constant region. For example, a variable region of the present invention is linked to a constant region shown below to form a heavy chain or a light chain of an antibody.

Heavy Chain Constant region linked downstream of
a humanized variable heavy region:
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK (SEQ ID
NO: 112)
Light Chain Constant region linked downstream of
a humanized variable light region:
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC (SEQ ID NO: 113)

Representative light chain and heavy chain sequences of the present invention are shown below (humanized variable regions derived from antibodies 81B4 and 73C5 linked to constant regions).

### Light Chain Amino Acid Sequences

>81B4vK32_3 Light Chain
EIVLTQSPGTLSLSPGERATMSCTASSSVSSSYFHWYQQKPGQAPRLLIYRTSTLASGI
PDRFSGSGSGTDFTLTISRLEPEDAATYYCHQFHRSPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 114)
>81B4vK32_105 Light Chain
EIVLTQSPGTLSLSPGERATMSCTASSSVSSSYFHWYQQKPGQAPRLLIYRTSILASGV
PDRFSGSGSGTDFTLTISRLEPEDFATYYCHQFHRSPLTFGQGTKLEIKRTVAAPSVFIF
PPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC (SEQ ID NO: 115)
>81B4vK32_116 Light Chain
EIVLTQSPGTLSLSPGERATMSCTASSSVSSSYFHWYQQKPGQAPRLWIYRTSRLASG
VPDRFSGSGSGTDFTLTISRLEPEDAATYYCHQFHRSPLTFGQGTKLEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 116)
>81B4vK32_127 Light Chain
EIVLTQSPGTLSLSPGERATMTCTASSSVSSSYFHWYQQKPGQAPRLLIYRTSRLASG
VPDRFSGSGSGTDFTLTISRLEPEDFAVYYCHQFHRSPLTFGQGTKLEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 117)
>81B4vK32_138 Light Chain
QIVLTQSPGTLSLSPGERATMTCTASSSVSSSYFHWYQQKPGQAPRLWIYRTSRLAS
GVPDRFSGSGSGTDFTLTISRLEPEDAATYYCHQFHRSPLTFGAGTKLEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC (SEQ ID NO: 118)
>81B4vK32_140 Light Chain
QIVLTQSPGTLSLSPGERVTMSCTASSSVSSSYFHWYQQKPGQAPRLLIYRTSQLASG
IPDRFSGSGSGTDFTLTISRLEPEDAATYYCHQFHRSPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 119)
>81 B4vK32_141 Light Chain
QIVLTQSPGTLSLSPGERATMTCTASSSVSSSYFHWYQQKPGQAPRLLIYRTSKLASG
VPDRFSGSGSGTDFTLTISRLEPEDFATYYCHQFHRSPLTFGQGTKLEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 120)
>81B4vK32_147 Light Chain
EIVLTQSPGTLSLSPGERATMSCTASSSVSSSYFHWYQQKPGQAPRLLIYRTSHLASGI
PGRFSGSGSGTDFTLTISRLEPEDAAVYYCHQFHRSPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 121)
>73C5vK39_2 Light Chain
EIVMTQSPATLSVSPGVRATLSCKASQDVGTNVLWYQQKPGQAPRPLIYSASYRHSGI
PDRFSGSGSGTEFTLTISSLQSEDFAEYFCQQYSRYPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 122)
>73C5vK39_7 Light Chain
EIVMTQSPATLSVSPGVRATLSCKASQDVGTNVLWYQQKPGQAPRPLIYSASYRHSGI
PDRFSGSGSGTEFTLTISSLOSEDFAVYYCQQYSRYPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 123)
>73C5vK39_15 Light Chain
EIVMTQSPATLSVSPGVRATLSCKASQDVGTNVLWYQQKPGQAPRPLIYSASYRHSGI
PARFSGSGSGTEFTLTISSLQSEDFAEYYCQQYSRYPLTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 124)

### Heavy Chain Amino Acid Sequences

>81B4vH33_49 Heavy Chain
(SEQ ID NO: 125)
QVQLVQSGAEVKKPGASVKVSCKASGYSFTSSWIHWVRQAPGQGLEWIGEINPGNV
RTNYNENFRNKATMTVDTSISTAYMELSRLRSDDTAVYYCAVVFYGEPYFPYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
>81B4vH33_85T Heavy Chain
(SEQ ID NO: 126)
QVQLVQSGAEVKKPGASVKVSCKASGYSFTSSWIHWVRQRPGQGLEWIGEINPGNV
RTNYNENFRNRVTMTVDTSISTAYMELSRLRSDDTAVYYCTVVFYGEPYFPYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
>81B4vH33_90 Heavy Chain
(SEQ ID NO: 127)
QVQLVQSGAEVKKPGASVKVSCKASGYSFTSSWIHWVKQAPGQGLEWMGEINPGNV
RTNYNENFRNKVTMTVDTSISTAYMELSRLRSDDTAVYYCTVVFYGEPYFPYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
>81B4vH33_93 Heavy Chain
(SEQ ID NO: 128)
QVQLVQSGAEVKKPGASVKVSCKASGYSFTSSWIHWVRQRPGQGLEWMGEINPGNV
RTNYNENFRNRATLTRDTSISTAYMELSRLRSDDTAVYYCAVVFYGEPYFPYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>81B4vH50_22 Heavy Chain
(SEQ ID NO: 129)
QVQLVQSGAEVKKPGASVKVSCKASGYSFTSSWIHWVRQRPGQGLEWMGEILPGVV
RTNYNENFRNKVTMTVDTSISTAYMELSRLRSDDTAVYYCTVVFYGEPYFPYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
>81B4vH50_30 Heavy Chain
(SEQ ID NO: 130)
QVQLVQSGAEVKKPGASVKVSCKASGYSFTSSWIHWVRQRPGQGLEWIGEINPGAV
RTNYNENFRNRVTMTVDTSISTAYMELSRLRSDDTAVYYCTVVFYGEPYFPYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
>81B4vH51_13 Heavy Chain
(SEQ ID NO: 131)
QVQLVQSGAEVKKPGASVKVSCKASGYSFTSSWIHWVRQAPGQGLEWIGEINPGLVR
TNYNENFRNKVTMTVDTSISTAYMELSRLRSDDTAVYYCAVVFYGEPYFPYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>81B4vH51_15 Heavy Chain
(SEQ ID NO: 132)
QVQLVQSGAEVKKPGASVKVSCKASGYSFTSSWIHWVRQAPGQGLEWIGEINPGAVR
TNYNENFRNKVTMTVDTSISTAYMELSRLRSDDTAVYYCAVVFYGEPYFPYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>81B4vH52_83 Heavy Chain
(SEQ ID NO: 133)
QVQLVQSGAEVKKPGASVKVSCKASGYSFTSSWIHWVRQAPGQGLEWIGEINPGSVR
TNYNENFRNKATMTVDTSISTAYMELSRLRSDDTAVYYCAVVFYGEPYFPYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>73C5vH46_4 Heavy Chain
(SEQ ID NO: 134)
QVQLQESGPGLVKPSETLSITCTVSGFSLTDYAVHWIRQPPGKGLEWIGVIWSDGSTD
YNAPFKSRVTINKDTSKSQVSFKMSSVQAADTAVYYCARKGGYSGSWFAYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>73C5vH46_19 Heavy Chain
(SEQ ID NO: 135)
QVQLQESGPGLVKPSETLSITCTVSGFSLTDYAVHWIRQPPGKGLEWIGVIWSDGSTD
YNAPFKSRVTISKDTSKNQVSLKMNSLTTDDTAVYYCARKGGYSGSWFAYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
>73C5vH46_40 Heavy Chain
(SEQ ID NO: 136)
QVQLQESGPGLVKPSETLSITCTVSGFSLTDYAVHWIRQPPGKGLEWIGVIWSDGSTD
YNAPFKSRVTISKDNSKSQVSLKMNSVTVADTAVYYCARKGGYSGSWFAYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>73C5vH47_65 Heavy Chain
(SEQ ID NO: 137)
QVQLQESGPGLVKPSETLSITCTVSGFSLTDYAVHWVRQPPGKGLEWIGVIWSDGST
DYNAPFKSRVTISKDTSKNQVSFKLSSVTVDDTAVYYCARKGGYSGSWFAYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>73C5vH47_77 Heavy Chain
(SEQ ID NO: 138)
QVQLQESGPGLVAPSETLSLTCTVSGFSLTDYAVHWIRQFPGKGLEWIGVIWSDGSTD
FNAPFKSRVTISKDTSKNQVSFKLSSVTTDDTAVYYCARKGGYSGSWFAYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPC
PAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
>73C5vH58_91 Heavy Chain
(SEQ ID NO: 139)
QVQLQESGPGLVKPSETLSITCTVSGFSLTDYAVHWIRQPPGKGLEWIGVIWSDGSTD
YNAPFKSRVTISKDNSKSQVSFKMSSVTADDTAVYYCARKGGYSGSWFAYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK

The CDRs listed above are defined using the Chothia numbering system (Al-Lazikani et al., (1997) JMB 273, 927-948).

In one aspect, an antibody of the present invention comprises 3 light chain CDRs and 3 heavy chain CDRs, for example as set forth above.

In one aspect, an antibody of the present invention comprises a light chain and a heavy chain variable region as set forth above. In one aspect, a light chain variable region of the invention is fused to a light chain constant region, for example a kappa or lambda constant region. In one aspect, a heavy chain variable region of the invention is fused to a heavy chain constant region, for example IgA, IgD, IgE, IgG or IgM, in particular, IgG₁, IgG₂, IgG₃ or IgG₄.

The present invention provides an anti-IL-36R antibody comprising a light chain comprising the amino acid sequence of SEQ ID NO: 115; and a heavy chain comprising the amino acid sequence of SEQ ID NO: 125 (Antibody B1).

The present invention provides an anti-IL-36R antibody comprising a light chain comprising the amino acid sequence of SEQ ID NO: 115; and a heavy chain comprising the amino acid sequence of SEQ ID NO: 126 (Antibody B2).

The present invention provides an anti-IL-36R antibody comprising a light chain comprising the amino acid sequence of SEQ ID NO: 115; and a heavy chain comprising the amino acid sequence of SEQ ID NO: 127 (Antibody B3).

The present invention provides an anti-IL-36R antibody comprising a light chain comprising the amino acid sequence of SEQ ID NO: 118; and a heavy chain comprising the amino acid sequence of SEQ ID NO: 125 (Antibody B4).

The present invention provides an anti-IL-36R antibody comprising a light chain comprising the amino acid sequence of SEQ ID NO: 118; and a heavy chain comprising the amino acid sequence of SEQ ID NO: 126 (Antibody B5).

The present invention provides an anti-IL-36R antibody comprising a light chain comprising the amino acid sequence of SEQ ID NO: 118; and a heavy chain comprising the amino acid sequence of SEQ ID NO: 127 Antibody B6).

The present invention provides an anti-IL-36R antibody comprising a light chain comprising the amino acid sequence of SEQ ID NO: 123; and a heavy chain comprising the amino acid sequence of SEQ ID NO: 138 (Antibody C3).

The present invention provides an anti-IL-36R antibody comprising a light chain comprising the amino acid sequence of SEQ ID NO: 123; and a heavy chain comprising the amino acid sequence of SEQ ID NO: 139 (Antibody C2).

The present invention provides an anti-IL-36R antibody comprising a light chain comprising the amino acid sequence of SEQ ID NO: 124; and a heavy chain comprising the amino acid sequence of SEQ ID NO: 138 (Antibody C1)

Representative antibodies of the present invention are shown below.

TABLE B

| Anti- | | |
|---|---|---|
| body | Light Chain Sequences | Heavy Chain Sequences |
| B1 | EIVLTQSPGTLSLSPGERATMSCT | QVQLVQSGAEVKKPGASVKVSCKASGY |
| | ASSSVSSSYFHWYQQKPGQAPR | SFTSSWIHWVRQAPGQGLEWIGEINPGN |
| | LLIYRTSILASGVPDRFSGSGSGT | VRTNYNENFRNKATMTVDTSISTAYMEL |
| | DFTLTISRLEPEDFATYYCHQFHR | SRLRSDDTAVYYCAVVFYGEPYFPYWG |
| | SPLTFGQGTKLEIKRTVAAPSVFIF | QGTLVTVSSASTKGPSVFPLAPSSKSTS |
| | PPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGPSVFLFPPKPK |
| | RGEC (SEQ ID NO: 115) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 125) |
| B2 | EIVLTQSPGTLSLSPGERATMSCT | QVQLVQSGAEVKKPGASVKVSCKASGY |
| | ASSSVSSSYFHWYQQKPGQAPR | SFTSSWIHWVRQRPGQGLEWIGEINPG |
| | LLIYRTSILASGVPDRFSGSGSGT | NVRTNYNENFRNRVTMTVDTSISTAYME |
| | DFTLTISRLEPEDFATYYCHQFHR | LSRLRSDDTAVYYCTVVFYGEPYFPYWG |
| | SPLTFGQGTKLEIKRTVAAPSVFIF | QGTLVTVSSASTKGPSVFPLAPSSKSTS |
| | PPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGSVFLFPPKPK |
| | RGEC (SEQ ID NO: 115) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 126) |
| B3 | EIVLTQSPGTLSLSPGERATMSCT | QVQLVQSGAEVKKPGASVKVSCKASGY |
| | ASSSVSSSYFHWYQQKPGQAPR | SFTSSWIHWVKQAPGQGLEWMGEINPG |
| | LLIYRTSILASGVPDRFSGSGSGT | NVRTNYNENFRNKVTMTVDTSISTAYME |
| | DFTLTISRLEPEDFATYYCHQFHR | LSRLRSDDTAVYYCTVVFYGEPYFPYWG |
| | SPLTFGQGTKLEIKRTVAAPSVFIF | QGTLVTVSSASTKGPSVFPLAPSSKSTS |
| | PPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGPSVFLFPPKPK |
| | RGEC (SEQ ID NO: 115) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 127) |
| B4 | QIVLTQSPGTLSLSPGERATMTCT | QVQLVQSGAEVKKPGASVKVSCKASGY |
| | ASSSVSSSYFHWYQQKPGQAPR | SFTSSWIHWVRQAPGQGLEWIGEINPGN |
| | LWIYRTSRLASGVPDRFSGSGSG | VRTNYNENFRNKATMTVDTSISTAYMEL |
| | TDFTLTISRLEPEDAATYYCHQFH | SRLRSDDTAVYYCAVVFYGEPYFPYWG |
| | QGTLVTVSSASTKGPSVFPLAPSSKSTS | RSPLTFGAGTKLEIKRTVAAPSVFI |
| | FPPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGPSVFLFPPKPK |
| | RGEC (SEQ ID NO: 118) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 125) |
| B5 | QIVLTQSPGTLSLSPGERATMTCT | QVQLVQSGAEVKKPGASVKVSCKASGY |
| | ASSSVSSSYFHWYQQKPGQAPR | SFTSSWIHWVRQRPGQGLEWIGEINPG |
| | LWIYRTSRLASGVPDRFSGSGSG | NVRTNYNENFRNRVTMTVDTSISTAYME |
| | TDFTLTISRLEPEDAATYYCHQFH | LSRLRSDDTAVYYCTVVFYGEPYFPYWG |
| | RSPLTFGAGTKLEIKRTVAAPSVFI | QGTLVTVSSASTKGPSVFPLAPSSKSTS |
| | FPPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGPSVFLFPPKPK |
| | RGEC (SEQ ID NO: 118) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 126) |
| B6 | QIVLTQSPGTLSLSPGERATMTCT | QVQLVQSGAEVKKPGASVKVSCKASGY |
| | ASSSVSSSYFHWYQQKPGQAPR | SFTSSWIHWVKQAPGQGLEWMGE NPG |
| | LWIYRTSRLASGVPDRFSGSGSG | NVRTNYNENFRNKVTMTVDTSISTAYME |
| | TDFTLTISRLEPEDAATYYCHQFH | LSRLRSDDTAVYYCTVVFYGEPYFPYWG |
| | RSPLTFGAGTKLEIKRTVAAPSVFI | QGTLVTVSSASTKGPSVFPLAPSSKSTS |
| | FPPSDEQLKSGTASVVCLLNNFYP | GGTAALGCLVKDYFPEPVTVSWNSGALT |
| | REAKVQWKVDNALQSGNSQESV | SGVHTFPAVLQSSGLYSLSSVVTVPSSS |
| | TEQDSKDSTYSLSSTLTLSKADYE | LGTQTYICNVNHKPSNTKVDKRVEPKSC |
| | KHKVYACEVTHQGLSSPVTKSFN | DKTHTCPPCPAPEAAGGPSVFLFPPKPK |
| | RGEC (SEQ ID NO: 118) | DTLMISRTPEVTCVVVDVSHEDPEVKFN |
| | | WYVDGVEVHNAKTKPREEQYNSTYRVV |
| | | SVLTVLHQDWLNGKEYKCKVSNKALPAP |
| | | IEKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 127) |

TABLE C

| Anti- | | |
|---|---|---|
| body | Light Chain Sequences | Heavy Chain Sequences |
| C1 | EIVMTQSPATLSVSPGVRATLSCK | QVQLQESGPGLVAPSETLSLTCTVSGFS |
| | ASQDVGTNVLWYQQKPGQAPRP | LTDYAVHWIRQFPGKGLEWIGVIWSDGS |
| | LIYSASYRHSGIPARFSGSGSGTE | TDFNAPFKSRVTISKDTSKNQVSFKLSSV |
| | FTLTISSLQSEDFAEYYCQQYSRY | TTDDTAVYYCARKGGYSGSWFAYWGQ |
| | PLTFGQGTKLEIKRTVAAPSVFIFP | GTLVTVSSASTKGPSVFPLAPSSKSTSG |
| | PSDEQLKSGTASVVCLLNNFYPR | GTAALGCLVKDYFPEPVTVSWNSGALTS |
| | EAKVQWKVDNALQSGNSQESVT | GVHTFPAVLQSSGLYSLSSVVTVPSSSL |
| | EQDSKDSTYSLSSTLTLSKADYEK | GTQTYICNVNHKPSNTKVDKRVEPKSCD |
| | HKVYACEVTHQGLSSPVTKSFNR | KTHTCPPCPAPEAAGGPSVFLFPPKPKD |
| | GEC (SEQ ID NO: 124) | TLMISRTPEVTCVVVDVSHEDPEVKFNW |
| | | YVDGVEVHNAKTKPREEQYNSTYRVVS |
| | | VLTVLHQDWLNGKEYKCKVSNKALPAPI |
| | | EKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 138) |
| C2 | EIVMTQSPATLSVSPGVRATLSCK | QVQLQESGPGLVKPSETLSITCTVSGFSL |
| | ASQDVGTNVLWYQQKPGQAPRP | TDYAVHWIRQPPGKGLEWIGVIWSDGST |
| | LIYSASYRHSGIPDRFSGSGSGTE | DYNAPFKSRVTISKDNSKSQVSFKMSSV |
| | FTLTISSLQSEDFAVYYCQQYSRY | TADDTAVYYCARKGGYSGSWFAYWGQ |
| | PLTFGQGTKLEIKRTVAAPSVFIFP | GTLVTVSSASTKGPSVFPLAPSSKSTSG |
| | PSDEQLKSGTASVVCLLNNFYPR | GTAALGCLVKDYFPEPVTVSWNSGALTS |
| | EAKVQWKVDNALQSGNSQESVT | GVHTFPAVLQSSGLYSLSSVVTVPSSSL |
| | EQDSKDSTYSLSSTLTLSKADYEK | GTQTYICNVNHKPSNTKVDKRVEPKSCD |
| | HKVYACEVTHQGLSSPVTKSFNR | KTHTCPPCPAPEAAGGPSVFLFPPKPKD |
| | GEC (SEQ ID NO: 123) | TLMISRTPEVTCVVVDVSHEDPEVKFNW |
| | | YVDGVEVHNAKTKPREEQYNSTYRVVS |
| | | VLTVLHQDWLNGKEYKCKVSNKALPAPI |
| | | EKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 139) |
| C3 | EIVMTQSPATLSVSPGVRATLSCK | QVQLQESGPGLVAPSETLSLTCTVSGFS |
| | ASQDVGTNVLWYQQKPGQAPRP | LTDYAVHWIRQFPGKGLEWIGVIWSDGS |
| | LIYSASYRHSGIPDRFSGSGSGTE | TDFNAPFKSRVTISKDTSKNQVSFKLSSV |
| | FTLTISSLQSEDFAVYYCQQYSRY | TTDDTAVYYCARKGGYSGSWFAYWGQ |
| | PLTFGQGTKLEIKRTVAAPSVFIFP | GTLVTVSSASTKGPSVFPLAPSSKSTSG |
| | PSDEQLKSGTASVVCLLNNFYPR | GTAALGCLVKDYFPEPVTVSWNSGALTS |
| | EAKVQWKVDNALQSGNSQESVT | GVHTFPAVLQSSGLYSLSSVVTVPSSSL |
| | EQDSKDSTYSLSSTLTLSKADYEK | GTQTYICNVNHKPSNTKVDKRVEPKSCD |
| | HKVYACEVTHQGLSSPVTKSFR | KTHTCPPCPAPEAAGGPSVFLFPPKPKD |
| | GEC (SEQ ID NO: 123) | TLMISRTPEVTCVVVDVSHEDPEVKFNW |
| | | YVDGVEVHNAKTKPREEQYNSTYRVVS |
| | | VLTVLHQDWLNGKEYKCKVSNKALPAPI |
| | | EKTISKAKGQPREPQVYTLPPSREEMTK |
| | | NQVSLTCLVKGFYPSDIAVEWESNGQPE |
| | | NNYKTTPPVLDSDGSFFLYSKLTVDKSR |
| | | WQQGNVFSCSVMHEALHNHYTQKSLSL |
| | | SPGK (SEQ ID NO: 138) |

In some aspects, the humanized antibody displays blocking activity, whereby it decreases the binding of IL-36 ligand to IL-36 receptor by at least 45%, by at least 50%, by at least 55%, by at least 60%, by at least 65%, by at least 70%, by at least 75%, by at least 80%, by at least 85%, by at least 90%, or by at least 95%. The ability of an antibody to block binding of IL-36 ligand to the IL-36 receptor can be measured using competitive binding assays known in the art. Alternatively, the blocking activity of an antibody can be measured by assessing the biological effects of IL-36, such as the production of IL-8, IL-6, and GM-CSF to determine if signaling mediated by the IL-36 receptor is inhibited.

In a further aspect, the present invention provides a humanized anti-IL-36R antibody having favorable biophysical properties. In one aspect, a humanized anti-IL-36R antibody of the present invention is present in at least 90% monomer form, or in at least 92% monomer form, or in at least 95% monomer form in a buffer. In a further aspect, a humanized anti-IL-36R antibody of the present invention remains in at least 90% monomer form, or in at least 92% monomer form, or in at least 95% monomer form in a buffer for one month or for four months.

In one aspect, a humanized antibody of the present invention is Antibody B1, Antibody B2, Antibody B3, Antibody B4, Antibody B5, Antibody B6, Antibody C1, Antibody C2, or Antibody C3. Accordingly, in one embodiment, a humanized antibody of the present invention comprises the light chain sequence of SEQ ID NO:115 and the heavy chain sequence of SEQ ID NO:125 (Antibody B1). In another embodiment, a humanized antibody of the present invention comprises the light chain sequence of SEQ ID NO:115 and the heavy chain sequence of SEQ ID NO:126 (Antibody B2). In another embodiment, a humanized antibody of the present invention comprises the light chain sequence of SEQ ID NO:115 and the heavy chain sequence of SEQ ID NO:127 (Antibody B3). In another embodiment, a humanized antibody of the present invention comprises the light chain sequence of SEQ ID NO:118 and the heavy chain sequence of SEQ ID NO:125 (Antibody B4). In another embodiment, a humanized antibody of the present invention comprises the light chain sequence of SEQ ID NO:118 and the heavy chain sequence of SEQ ID NO:126 (Antibody B5). In another embodiment, a humanized antibody of the present invention comprises the light chain sequence of SEQ ID NO:118 and the heavy chain sequence of SEQ ID NO:127 (Antibody B6). In another embodiment, a humanized antibody of the present invention comprises the light chain sequence of SEQ ID NO:124 and the heavy chain sequence of SEQ ID NO:138 (Antibody C1). In another embodiment, a humanized antibody of the present invention comprises the light chain sequence of SEQ ID NO:123 and the heavy chain sequence of SEQ ID NO:139 (Antibody C2). In another embodiment, a humanized antibody of the present invention comprises the light chain sequence of SEQ ID NO:123 and the heavy chain sequence of SEQ ID NO:138 (Antibody C3).

In a further embodiment, a humanized antibody of the present invention consists of the light chain sequence of SEQ ID NO:115 and the heavy chain sequence of SEQ ID NO:125 (Antibody B1). In another embodiment, a humanized antibody of the present invention consists of the light chain sequence of SEQ ID NO:115 and the heavy chain sequence of SEQ ID NO:126 (Antibody B2). In another embodiment, a humanized antibody of the present invention consists of the light chain sequence of SEQ ID NO:115 and the heavy chain sequence of SEQ ID NO:127 (Antibody B3). In another embodiment, a humanized antibody of the present invention consists of the light chain sequence of SEQ ID NO:118 and the heavy chain sequence of SEQ ID NO:125 (Antibody B4). In another embodiment, a humanized antibody of the present invention consists of the light chain sequence of SEQ ID NO:118 and the heavy chain sequence of SEQ ID NO:126 (Antibody B5). In another embodiment, a humanized antibody of the present invention consists of the light chain sequence of SEQ ID NO:118 and the heavy chain sequence of SEQ ID NO:127 (Antibody B6). In another embodiment, a humanized antibody of the present invention consists of the light chain sequence of SEQ ID NO:124 and the heavy chain sequence of SEQ ID NO:138 (Antibody C1). In another embodiment, a humanized antibody of the present invention consists of the light chain sequence of SEQ ID NO:123 and the heavy chain sequence of SEQ ID NO:139 (Antibody C2). In another embodiment, a humanized antibody of the present invention consists of the light chain sequence of SEQ ID NO:123 and the heavy chain sequence of SEQ ID NO:138 (Antibody C3).

In some embodiments, the humanized anti-IL-36R antibodies, including antigen-binding fragments thereof, such as heavy and light chain variable regions, comprise an amino acid sequence of the residues derived from Antibody B1, Antibody B2, Antibody B3, Antibody B4, Antibody B5, Antibody B6, Antibody C1, Antibody C2, or Antibody C3.

In a further embodiment, the present invention provides an anti-IL-36R antibody or antigen-binding fragment thereof that competitively binds to human anti-IL-36R with an antibody of the present invention, for example Antibody B1, Antibody B2, Antibody B3, Antibody B4, Antibody B5, Antibody B6, Antibody C1, Antibody C2 or Antibody C3 described herein. The ability of an antibody or antigen-binding fragment to competitively bind to IL-36R can be measured using competitive binding assays known in the art.

The humanized anti-IL-36R antibodies optionally include specific amino acid substitutions in the consensus or germline framework regions. The specific substitution of amino acid residues in these framework positions can improve various aspects of antibody performance including binding affinity and/or stability, over that demonstrated in humanized antibodies formed by “direct swap” of CDRs or HVLs into the human germline framework regions.

In some embodiments, the present invention describes other monoclonal antibodies with a light chain variable region having the amino acid sequence set forth in any one of SEQ ID NO:1-10. In some embodiments, the present invention describes other monoclonal antibodies with a heavy chain variable region having the amino acid sequence set forth in any one of SEQ ID NO:11-20. Placing such CDRs into FRs of the human consensus heavy and light chain variable domains will yield useful humanized antibodies of the present invention.

In particular, the present invention provides monoclonal antibodies with the combinations of light chain variable and heavy chain variable regions of SEQ ID NO:1/11, 2/12, 3/13, 4/14, 5/15, 6/16, 7/17, 8/18, 9/19, 10/20. Such variable regions can be combined with human constant regions.

In some embodiments, the present invention describes other humanized antibodies with light chain variable region sequences having the amino acid sequence set forth in any one of SEQ ID NO:76-86. In some embodiments, the present invention describes other humanized antibodies with heavy chain variable region sequences having the amino acid sequence set forth in any one of SEQ ID NO:87-101. In particular, the present invention provides monoclonal antibodies with the combinations of light chain variable and heavy chain variable regions of SEQ ID NO: 77/89, 80/88, 80/89, 77/87, 77/88, 80/87, 86/100, 85/101, 85/100. Such variable regions can be combined with human constant regions.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:89 and framework regions having an amino acid sequence at least 90% identical, at least 93% identical or at least 95% identical to the amino acid sequence of the framework regions of the variable domain heavy chain amino acid sequence of SEQ ID NO:89. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:88 and framework regions having an amino acid sequence at least 90% identical, at least 93% identical or at least 95% identical to the amino acid sequence of the framework regions of the variable domain heavy chain amino acid sequence of SEQ ID NO:88. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:89 and framework regions having an amino acid sequence at least 90% identical, at least 93% identical or at least 95% identical to the amino acid sequence of the framework regions of the variable domain heavy chain amino acid sequence of SEQ ID NO:89. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:87 and framework regions having an amino acid sequence at least 90% identical, at least 93% identical or at least 95% identical to the amino acid sequence of the framework regions of the variable domain heavy chain amino acid sequence of SEQ ID NO:87. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:77 and framework regions having an amino acid sequence at least 90% identical, at least 93% identical or at least 95% identical to the amino acid sequence of the framework regions of the variable domain light chain amino acid sequence of SEQ ID NO:77 and a humanized heavy chain variable domain comprising the CDRs of SEQ ID NO:88 and framework regions having an amino acid sequence at least 90% identical, at least 93% identical or at least 95% identical to the amino acid sequence of the framework regions of the variable domain heavy chain amino acid sequence of SEQ ID NO:88. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:80 and framework regions having an amino acid sequence at least 90% identical, at least 93% identical or at least 95% identical to the amino acid sequence of the framework regions of the variable domain light chain amino acid sequence of SEQ ID NO:80 and a humanized heavy chain variable domain comprising the CDRs of SEQ ID NO:87 and framework regions having an amino acid sequence at least 90% identical, at least 93% identical or at least 95% identical to the amino acid sequence of the framework regions of the variable domain heavy chain amino acid sequence of SEQ ID NO:87. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:86 and framework regions having an amino acid sequence at least 90% identical, at least 93% identical or at least 95% identical to the amino acid sequence of the framework regions of the variable domain light chain amino acid sequence of SEQ ID NO:86 and a humanized heavy chain variable domain comprising the CDRs of SEQ ID NO:100 and framework regions having an amino acid sequence at least 90% identical, at least 93% identical or at least 95% identical to the amino acid sequence of the framework regions of the variable domain heavy chain amino acid sequence of SEQ ID NO:100. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:85 and framework regions having an amino acid sequence at least 90% identical, at least 93% identical or at least 95% identical to the amino acid sequence of the framework regions of the variable domain light chain amino acid sequence of SEQ ID NO:85 and a humanized heavy chain variable domain comprising the CDRs of SEQ ID NO:101 and framework regions having an amino acid sequence at least 90% identical, at least 93% identical or at least 95% identical to the amino acid sequence of the framework regions of the variable domain heavy chain amino acid sequence of SEQ ID NO:101. In one embodiment, the anti-IL-36R antibody is a humanized monoclonal antibody.

In a further embodiment, the present invention relates to an anti-IL-36R antibody or antigen-binding fragment thereof comprising a humanized light chain variable domain comprising the CDRs of SEQ ID NO:85 and framework regions having an amino acid sequence at least 90% identical, at least 93% identical or at least 95% identical to the amino acid sequence of the framework regions of the variable domain light chain amino acid sequence of SEQ ID NO:85 and a humanized heavy chain variable domain comprising the CDRs of SEQ ID NO:100 and framework regions having an amino acid sequence at least 90% identical, at least 93% identical or at least 95% identical to the amino acid sequence of the framework regions of the variable domain heavy chain amino acid sequence of SEQ ID NO:100. In one embodiment, the anti-IL-36R antibody is a humanized monoclonal antibody.

In some specific embodiments, the humanized anti-IL-36R antibodies disclosed herein comprise at least a heavy or a light chain variable domain comprising the CDRs or HVLs of the murine monoclonal antibodies or humanized antibodies as disclosed herein and the FRs of the human germline heavy and light chain variable domains.

In one further aspect, the present invention provides an anti-IL-36R antibody or antigen-binding fragment thereof comprising a light chain CDR1 (L-CDR1) sequence of any one of SEQ ID NO:21-29; a light chain CDR2 (L-CDR2) sequence of any one of SEQ ID NO:30-38; a light chain CDR3 (L-CDR3) sequence of any one of SEQ ID NO:39-47; a heavy chain CDR1 (H-CDR1) sequence of any one of SEQ ID NO:48-56; a heavy chain CDR2 (H-CDR2) sequence of any one of SEQ ID NO:57-66; and a heavy chain CDR3 (H-CDR3) sequence of any one of SEQ ID NO:67-75. In one aspect, the anti-IL-36R antibody or antigen-binding fragment thereof comprises a light chain variable region comprising a L-CDR1 listed above, a L-CDR2 listed above and a L-CDR3 listed above, and a heavy chain variable region comprising a H-CDR1 listed above, a H-CDR2 listed above and a H-CDR3 listed above.

In a further aspect, the present invention provides an anti-IL-36R antibody or antigen-binding fragment thereof comprising:
a) a L-CDR1, a L-CDR2, a L-CDR3, a H-CDR1, a H-CDR2 and a H-CDR3 sequence of SEQ ID NO:21, 30, 39, 48, 57 and 67, respectively; or
b) a L-CDR1, a L-CDR2, a L-CDR3, a H-CDR1, a H-CDR2 and a H-CDR3 sequence of SEQ ID NO:22, 31, 40, 49, 58 and 68, respectively; or
c) a L-CDR1, a L-CDR2, a L-CDR3, a H-CDR1, a H-CDR2 and a H-CDR3 sequence of SEQ ID NO:23, 32, 41, 50, 59 and 69, respectively; or
d) a L-CDR1, a L-CDR2, a L-CDR3, a H-CDR1, a H-CDR2 and a H-CDR3 sequence of SEQ ID NO:24, 33, 42, 51, 60 and 70, respectively; or
e) a L-CDR1, a L-CDR2, a L-CDR3, a H-CDR1, a H-CDR2 and a H-CDR3 sequence of SEQ ID NO:25, 34, 43, 52, 61 and 71, respectively; or
f) a L-CDR1, a L-CDR2, a L-CDR3, a H-CDR1, a H-CDR2 and a H-CDR3 sequence of SEQ ID NO:26, 35, 44, 53, 62 and 72, respectively; or
g) a L-CDR1, a L-CDR2, a L-CDR3, a H-CDR1, a H-CDR2 and a H-CDR3 sequence of SEQ ID NO:27, 36, 45, 54, 63 and 73, respectively; or
h) a L-CDR1, a L-CDR2, a L-CDR3, a H-CDR1, a H-CDR2 and a H-CDR3 sequence of SEQ ID NO:27, 36, 45, 54, 64 and 74, respectively; or
i) a L-CDR1, a L-CDR2, a L-CDR3, a H-CDR1, a H-CDR2 and a H-CDR3 sequence of SEQ ID NO:27, 36, 45, 54, 64 and 73, respectively; or
j) a L-CDR1, a L-CDR2, a L-CDR3, a H-CDR1, a H-CDR2 and a H-CDR3 sequence of SEQ ID NO:28, 37, 46, 55, 65 and 74, respectively; or
k) a L-CDR1, a L-CDR2, a L-CDR3, a H-CDR1, a H-CDR2 and a H-CDR3 sequence of SEQ ID NO:29, 38, 47, 56, 66 and 75, respectively.

In a further aspect, the present invention provides an anti-IL-36R antibody or antigen-binding fragment thereof comprising:
a) a L-CDR1, a L-CDR2, a L-CDR3, a H-CDR1, a H-CDR2 and a H-CDR3 sequence of SEQ ID NO:26, 103, 44, 53, 62 and 72, respectively; or
b) a L-CDR1, a L-CDR2, a L-CDR3, a H-CDR1, a H-CDR2 and a H-CDR3 sequence of SEQ ID NO:26, 104, 44, 53, 62 and 72, respectively; or
c) a L-CDR1, a L-CDR2, a L-CDR3, a H-CDR1, a H-CDR2 and a H-CDR3 sequence of SEQ ID NO:27, 36, 45, 107, 63 and 73, respectively; or
d) a L-CDR1, a L-CDR2, a L-CDR3, a H-CDR1, a H-CDR2 and a H-CDR3 sequence of SEQ ID NO:27, 36, 45, 107, 64 or 73, respectively.

In one aspect, the anti-IL-36R antibody or antigen-binding fragment thereof comprises a light chain variable region comprising a L-CDR1, L-CDR2 and L-CDR3 combination listed above, and a heavy chain variable region comprising a H-CDR1, H-CDR2 and H-CDR3 combination listed above.

In specific embodiments, it is contemplated that chimeric antibodies with switched CDR regions (i.e., for example switching one or two CDRs of one of the mouse antibodies or humanized antibody derived therefrom with the analogous CDR from another mouse antibody or humanized antibody derived therefrom) between these exemplary immunoglobulins may yield useful antibodies.

In certain embodiments, the humanized anti-IL-36R antibody is an antibody fragment. Various antibody fragments have been generally discussed above and there are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)2 fragments (see, e.g., Carter et al., 1992, Bio/Technology 10:163-167). By another approach, F(ab′)2 fragments can be isolated directly from recombinant host cell culture. Other techniques for the production of antibody fragments will be apparent to the skilled practitioner. Accordingly, in one aspect, the present invention provides antibody fragments comprising the CDRs described herein, in particular one of the combinations of L-CDR1, L-CDR2, L-CDR3, H-CDR1, H-CDR2 and H-CDR3 described herein. In a further aspect, the present invention provides antibody fragments comprising the variable regions described herein, for example one of the combinations of light chain variable regions and heavy chain variable regions described herein.

Certain embodiments include an F(ab′)₂ fragment of a humanized anti-IL-36R antibody comprise a light chain sequence of any of SEQ ID NO: 115 or 118 in combination with a heavy chain sequence of SEQ ID NO: 125, 126 or 127. Such embodiments can include an intact antibody comprising such an F(ab′)₂.

Certain embodiments include an F(ab′)₂ fragment of a humanized anti-IL-36R antibody comprise a light chain sequence of any of SEQ ID NO: 123 or 124 in combination with a heavy chain sequence of SEQ ID NO: 138 or 139. Such embodiments can include an intact antibody comprising such an F(ab′)2.

In some embodiments, the antibody or antibody fragment includes a constant region that mediates effector function. The constant region can provide antibody-dependent cellular cytotoxicity (ADCC), antibody-dependent cellular phagocytosis (ADCP) and/or complement-dependent cytotoxicity (CDC) responses against an anti-IL-36R expressing target cell. The effector domain(s) can be, for example, an Fc region of an Ig molecule.

The effector domain of an antibody can be from any suitable vertebrate animal species and isotypes. The isotypes from different animal species differ in the abilities to mediate effector functions. For example, the ability of human immunoglobulin to mediate CDC and ADCC/ADCP is generally in the order of IgM≈IgG₁≈IgG₂>IgG₄ and IgG₁≈IgG₃>IgG₂/IgM/IgG₄, respectively. Murine immunoglobulins mediate CDC and ADCC/ADCP generally in the order of murine IgM≈IgG₃>>IgG_{2b}>IgG₂ₐ>>IgG₁ and IgG_{2b}>IgG₂ₐ>IgG₁>>IgG₃, respectively. In another example, murine IgG2a mediates ADCC while both murine IgG2a and IgM mediate CDC.

### III. Pharmaceutical Doses and Administration

Anti-IL-36R antibodies of the present invention are typically administered to a patient as a pharmaceutical composition in which the antagonist is admixed with a pharmaceutically acceptable carrier or excipient, see, e. g., Remington's Pharmaceutical Sciences and US. Pharmacopeia: National Formulary, Mack Publishing Company, Easton, Pa. (1984). The pharmaceutical composition may be formulated in any manner suitable for the intended route of administration. Examples of pharmaceutical formulations include lyophilized powders, slurries, aqueous solutions, suspensions and sustained release formulations (see, e. g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). Suitable routes of administration include intravenous injection (including intraarterial injection) and subcutaneous injection.

Representative examples of doses and dose regimens according to the present invention are disclosed in Table A. Although, doses 900 mg and 750 mg have been exemplified, similar dose regimens equally apply to doses 210 mg, 300 mg, 350 mg, 450 mg, 600 mg, 700 mg and 800 mg.

TABLE A
Doses and Dose Regimens

| | | | Optional | | |
|---|---|---|---|---|---|
| Intravenous | Frequency of | Frequency of | Subcutaneous | Frequency of | Frequency of |
| dose | intravenous | intravenous | dose | subcutaneous | subcutaneous |
| (mg) | doses | administration | (mg) | doses | administration |
| 900 | 2 | Second dose 1 week | — | — | — |
| | | after the first dose | | | |
| 900 | 2 | Second dose 1 week | 150 | 1 or more | At 2, 4, 6, 8, 10 or |
| | | after the first dose | | | 12 weeks |
| | | | | | intervals |
| 900 | 2 | Second dose 1 week | 150 | 2 or more | At 2, 4, 6, 8, 10 or |
| | | after the first dose | | | 12 weeks |
| | | | | | intervals |
| 900 | 2 | Second dose 1 week | 150 | 3 or more | At 2, 4, 6, 8, 10 or |
| | | after the first dose | | | 12 weeks |
| | | | | | intervals |
| 900 | 2 | Second dose 1 week | — | — | — |
| | | after the first dose | | | |
| 900 | 2 | Second dose 1 week | 225 | 1 or more | At 2, 4, 6, 8, 10 or |
| | | after the first dose | | | 12 weeks |
| | | | | | intervals |
| 900 | 2 | Second dose 1 week | 225 | 2 or more | At 2, 4, 6, 8, 10 or |
| | | after the first dose | | | 12 weeks |
| | | | | | intervals |
| 900 | 2 | Second dose 1 week | 225 | 3 or more | At 2, 4, 6, 8, 10 or |
| | | after the first dose | | | 12 weeks |
| | | | | | intervals |
| 900 | 2 | Second dose 1 week | 300 | 1 or more | At 2, 4, 6, 8, 10 or |
| | | after the first dose | | | 12 weeks |
| | | | | | intervals |
| 900 | 2 | Second dose 1 week | 300 | 2 or more | At 2, 4, 6, 8, 10 or |
| | | after the first dose | | | 12 weeks |
| | | | | | intervals |
| 900 | 2 | Second dose 1 week | 300 | 3 or more | At 2, 4, 6, 8, 10 or |
| | | after the first dose | | | 12 weeks |
| | | | | | intervals |
| 900 | 2 | Second dose 1 week | 600 | 1 or more | At 2, 4, 6, 8, 10 or |
| | | after the first dose | | | 12 weeks |
| | | | | | intervals |
| 900 | 2 | Second dose 1 week | 600 | 2 or more | At 2, 4, 6, 8, 10 or |
| | | after the first dose | | | 12 weeks |
| | | | | | intervals |
| 900 | 2 | Second dose 1 week | 600 | 3 or more | At 2, 4, 6, 8, 10 or |
| | | after the first dose | | | 12 weeks |
| | | | | | intervals |
| 900 | 3 | Second dose 1 week | — | — | — |
| | | after the first dose; | | | |
| | | third dose 2, 4, 6, 8, | | | |
| | | 10 or 12 weeks after | | | |
| | | the second dose | | | |
| 900 | 3 | Second dose 1 week | 150 | 1 or more | At 2, 4, 6, 8, 10 or |
| | | after the first dose; | | | 12 weeks |
| | | third dose 1, 2, 4, 6, | | | intervals |
| | | 8, 10 or 12 weeks after | | | |
| | | the second dose | | | |
| 900 | 3 | Second dose 1 week | 150 | 2 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 150 | 3 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 225 | 1 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 225 | 2 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 225 | 3 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 300 | 1 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 300 | 2 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 300 | 3 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 600 | 1 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 600 | 2 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |
| 900 | 3 | Second dose 1 week | 600 | 3 or more | At 2, 4, 6, 8, 10 or |
| | | after the first dose; | | | 12 weeks |
| | | third dose 2, 4, 6, 8, | | | intervals |
| | | 10 or 12 weeks after | | | |
| | | the second dose | | | |

In one embodiment, 1, 2 or 3 or more intravenous dose(s) is/are administered to the patient in a dose regimen listed in Table A, wherein a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.

In one aspect, the invention relates to the treatment of GPP in a patient by administering to the patient two 900 mg intravenous (i.v.) doses of an anti-IL-36R antibody; wherein the second dose is administered less than 2 weeks after the first dose.

In another aspect, the invention relates to the treatment of GPP in a patient by administering to the patient a single 900 mg intravenous dose of an anti-IL-36R antibody, if the GPP symptoms persist, administering an additional 900 mg intravenous dose an anti-IL-36R antibody one week after the initial dose.

In one aspect, the invention relates to a method of treating generalized pustular psoriasis (GPP) flares in a patient, said method comprising administering to the patient two 900 mg intravenous (i.v.) doses of an anti-IL-36R antibody; wherein the second dose is administered less than 2 weeks after the first dose.

In one aspect, the invention relates to a method of treating GPP in a patient, said method comprising administering to the patient two 900 mg intravenous doses of an anti-IL-36R antibody; wherein the second dose is administered less than 2 weeks after the first dose.

In one aspect, the invention relates to a method of reducing or alleviating signs and symptoms of an acute phase flare-up of GPP in a patient, said method comprising administering to the patient two 900 mg intravenous doses of an anti-IL-36R antibody; wherein the second dose is administered less than 2 weeks after the first dose.

In one aspect, the invention relates to a method of reducing the severity and duration of GPP symptoms, said method comprising including administering to the patient two 900 mg intravenous doses of an anti-IL-36R antibody; wherein the second dose is administered less than 2 weeks after the first dose.

In one aspect, the invention relates to a method of treating a skin disorder associated with GPP, said method comprising administering to the patient two 900 mg intravenous doses of an anti-IL-36R antibody; wherein the second dose is administered less than 2 weeks after the first dose.

In one aspect, the invention relates to a method of preventing the recurrence of GPP flares in a patient, said method comprising administering to the patient two 900 mg intravenous doses of an anti-IL-36R antibody; wherein the second dose is administered less than 2 weeks after the first dose.

In one aspect, the invention relates to a method of reducing pain by at least 10% in a patient with GPP, said method comprising administering to the patient two 900 mg intravenous doses of an anti-IL-36R antibody; wherein the second dose is administered less than 2 weeks after the first dose.

In one aspect, the invention relates to a method of improving the quality of life by at least 10% in a patient with moderate to severe GPP symptoms, said method comprising administering to the patient two 900 mg intravenous doses of an anti-IL-36R antibody; wherein the second dose is administered less than 2 weeks after the first dose.

In an embodiment relating to any of the above aspects, the patient has a GPP Physician Global Assessment (GPPGA) total score of ≥2.

In an embodiment relating to any of the above aspects, the patient has a GPP Physician Global Assessment (GPPGA) total score of ≥2 and a GPPGA pustulation subscore of ≥2.

In an embodiment relating to any of the above aspects, the patient has a GPP Physician Global Assessment (GPPGA) total score of ≥2 and a GPPGA pustulation subscore of ≥2 before the administration of the first i.v. dose.

In an embodiment relating to any of the above aspects, the patient has a GPP Physician Global Assessment (GPPGA) total score of ≥2 and a GPPGA pustulation subscore of ≥2 after the administration of the first i.v. dose.

In an embodiment relating to any of the above aspects, the patient has a GPP Physician Global Assessment (GPPGA) total score of ≥2 and a GPPGA pustulation subscore of ≥2 before and after the administration of the first i.v. dose.

In an embodiment relating to any of the above aspects, the second dose is administered after 1 week but less than 2 weeks from the first dose.

In one aspect, the invention relates to a method of treating a GPP patient with a GPPGA pustulation subscore of ≥2, said method comprising the steps of: (a) administering to the patient a first 900 mg intravenous (I.V.) dose of an anti-IL-36R antibody; (b) assessing the GPPGA pustulation subscore of the patient and if the GPPGA pustulation subscore of ≥2 of the patient persists after 1 week from the first dose, then administering to the patient a second 900 mg (i.v.) dose of spesolimab less than 2 weeks after the first dose.

In one aspect, the invention relates to a method of treating a GPP patient with a GPP Physician Global Assessment (GPPGA) total score of ≥2, said method comprising the steps of: (a) administering to the patient a first 900 mg intravenous (i.v.) dose of an anti-IL-36R antibody; (b) assessing the GPPGA total score of the patient and if the GPPGA total score of ≥2 of the patient persists after 1 week from the first dose, then administering to the patient a second 900 mg (i.v.) dose of spesolimab less than 2 weeks after the first dose.

In one aspect, the invention relates to a method of treating a GPP patient with a GPP Physician Global Assessment (GPPGA) total score of ≥2 and a GPPGA pustulation subscore of ≥2, said method comprising the steps of: (a) administering to the patient a first 900 mg intravenous (i.v.) dose of an anti-IL-36R antibody; (b) assessing the GPPGA scores of the patient and if the GPPGA total score of ≥2 and the GPPGA pustulation subscore of ≥2 of the patient persist after 1 week from the first dose, then administering to the patient a second 900 mg (i.v.) dose of spesolimab less than 2 weeks after the first dose.

In an embodiment relating to any of the above aspects or embodiments, an optional third 900 mg i.v. dose of the anti-IL-36R antibody is administered at 2 to 12 weeks after the second i.v. dose.

In an embodiment relating to any of the above aspects or embodiments, the two-dose administration achieves one or more of the following results: (a) a Generalized Pustular Psoriasis Global Assessment (GPPGA) pustulation subscore of 0 indicating in one week after administering the second i.v. dose; and/or (b) a GPPGA total score of 0 or 1 in one week after administering the second i.v. dose.

In an embodiment relating to any of the above aspects or embodiments, the results are maintained for up to and at least 12 weeks following the administration of the second i.v. dose.

In an embodiment relating to any of the above aspects or embodiments, the method comprises administering to the patient a prophylactically effective amount of the anti-IL-36R antibody in one or more subcutaneous doses after the last i.v. dose administered.

In an embodiment relating to any of the above aspects or embodiments, each of the one or more subcutaneous doses comprises 150 mg, 225 mg, 300 mg, 450 mg, or 600 mg of said anti-IL-36R antibody.

In an embodiment relating to any of the above aspects or embodiments, 1, 2, 3 or more subcutaneous doses are administered to the patient and wherein a first subcutaneous dose is administered after the last intravenous dose.

In an embodiment relating to any of the above aspects or embodiments, a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.

In an embodiment relating to any of the above aspects or embodiments, the patient remains in clinical remission as measured by a GPPGA total score of 0 or 1 for at least 12, 24, 36, 48, 60 or 72 weeks following the last i.v. or subcutaneous dose.

In an embodiment relating to any of the above aspects or embodiments, the mammal or the patient is evaluated for improved Clinical Remission as defined by: (a) Generalized Pustular Psoriasis Global Assessment (GPPGA) score of 0 or 1 at Week 1; (b) GPPGA pustulation subscore of 0 indicating no visible pustules at Week 1; (c) Psoriasis Area and Severity Index for Generalized Pustular Psoriasis (GPPASI) 75 at Week 4; (d) Change from baseline in Pain Visual Analog Scale (VAS) score at Week 4; (e) Change from baseline in Psoriasis Symptom Scale (PSS) score at Week 4; (f) Change from baseline in Functional Assessment of Chronic Illness Therapy (FACIT) Fatigue score at Week 4; (g) GPPGA 0 or 1 at Week 4; (h) GPPGA pustulation subscore of 0 indicating no visible pustules at Week 4; (i) GPPASI 50 at Week 1 and 4; or (j) Change in GPPASI pustule, erythema or scaling severity subscore from baseline at Week 1 and 4. In a related embodiment, proportion of patients with a response to the administration is statistically significantly higher as compared to patients on placebo for any of the end points recited.

### Pharmaceutical Compositions and Administration Thereof

The antibodies of the present invention can be administered either alone or in combination with other agents. Examples of antibodies for use in such pharmaceutical compositions are those that comprise an antibody or antibody fragment having the light chain variable region amino acid sequence of any of SEQ ID NO: 1-10. Examples of antibodies for use in such pharmaceutical compositions are also those that comprise a humanized antibody or antibody fragment having the heavy chain variable region amino acid sequence of any of SEQ ID NO: 11-20.

Further examples of antibodies for use in such pharmaceutical compositions are also those that comprise a humanized antibody or antibody fragment having the light chain variable region amino acid sequence of any of SEQ ID NO:76-86. Preferred antibodies for use in such pharmaceutical compositions are also those that comprise a humanized antibody or antibody fragment having the heavy chain variable region amino acid sequence of any of SEQ ID NO:87-101.

Further examples of antibodies for use in such pharmaceutical compositions are also those that comprise a humanized antibody or antibody fragment having the light chain variable region and heavy chain variable region of any of SEQ ID NO: 77 and 89, SEQ ID NO: 80 and 88, SEQ ID NO: 80 and 89, SEQ ID NO: 77 and 87, SEQ ID NO: 77 and 88, SEQ ID NO: 80 and 87, SEQ ID NO: 86 and 100, SEQ ID NO: 85 and 101, or SEQ ID NO: 85 and 10.

Further examples of antibodies for use in such pharmaceutical compositions are also those that comprise a humanized antibody having the light chain region amino acid sequence of any of SEQ ID NO:115, 118, 123 or 124. Preferred antibodies for use in such pharmaceutical compositions are also those that comprise humanized antibody having the heavy chain variable region amino acid sequence of any of SEQ ID NO:125, 126, 127, 138 or 139.

Further examples of antibodies for use in such pharmaceutical compositions are also those that comprise Antibody B1, Antibody B2, Antibody B3, Antibody B4, Antibody B5, Antibody B6, Antibody C1, Antibody C2 or Antibody C3.

Various delivery systems are known and can be used to administer the IL-36R binding agent. Methods of introduction include but are not limited to intradermal, intramuscular, intraperitoneal, intravenous, subcutaneous, intranasal, epidural, and oral routes. The IL-36R binding agent can be administered, for example by infusion, bolus or injection, and can be administered together with other biologically active agents such as chemotherapeutic agents. Administration can be systemic or local. In preferred embodiments, the administration is by subcutaneous injection. Formulations for such injections may be prepared in for example prefilled syringes that may be administered once every other week.

In one aspect, the invention provides an article of manufacture comprising a subcutaneous administration device, which delivers to a patient a fixed dose of an antibody of the present invention. In some embodiments, the subcutaneous administration device is a pre-filled syringe, an autoinjector, or a large volume infusion device. For example, MyDose™ product from Roche, a single use infusion device that enables the subcutaneous administration of large quantities of liquid medication, may be used as the administration device. Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park III.), YPSOMATE™, YPSOMATE 2.25™, VAIROJECT™ (Ypsomed AG, Burgdorf, Switzerland) to name only a few. Additional information relating to example delivery devices that could be used with an antibody of the present invention may be found, for example, in CH705992A2, WO2009/040602, WO2016/169748, WO2016/179713.

In specific embodiments, the IL-36R binding agent composition is administered by injection, by means of a catheter, by means of a suppository, or by means of an implant, the implant being of a porous, non-porous, or gelatinous material, including a membrane, such as a silastic membrane, or a fiber. Typically, when administering the composition, materials to which the anti-IL-36R antibody or agent does not absorb are used.

In other embodiments, the anti-IL-36R antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An IL-36R binding agent (e.g., an anti-IL-36R antibody) can be administered as pharmaceutical compositions comprising a therapeutically effective amount of the binding agent and one or more pharmaceutically compatible ingredients.

In one embodiment, the anti-IL-36R antibody or an antigen binding fragment thereof (disclosed herein) is present in a pharmaceutical formulation (as described in co-pending PCT publication No. 20200185479, published on Sep. 17, 2020, the entire content of which is hereby incorporated herein by reference in its entirety) suitable for administration to a mammal or patient according to any one of the aspects described herein. Various examples to this embodiment are described as numbered clauses (1, 2, 3, etc.) below for convenience. These are provided as examples and do not limit the subject technology. It is noted that any of the dependent clauses may be combined in any combination, and placed into a respective independent clause, e.g., clause 1. The other clauses can be presented in a similar manner.
1. A method of treating generalized pustular psoriasis (GPP) flares in a patient, said method comprising administering to the patient two 900 mg intravenous (i.v.) doses of an anti-IL-36R antibody; wherein the second dose is administered less than 2 weeks after the first dose.
2. A method of treating GPP in a patient by administering to the patient a single 900 mg intravenous dose of an anti-IL-36R antibody, if the GPP symptoms persist, administering an additional 900 mg intravenous dose an anti-IL-36R antibody one week after the initial dose.
3. A method of treating GPP in a patient, said method comprising administering to the patient two 900 mg intravenous doses of an anti-IL-36R antibody; wherein the second dose is administered less than 2 weeks after the first dose.
4. A method of reducing or alleviating signs and symptoms of an acute phase flare-up of GPP in a patient, said method comprising administering to the patient two 900 mg intravenous doses of an anti-IL-36R antibody; wherein the second dose is administered less than 2 weeks after the first dose.
5. A method of reducing the severity and duration of GPP symptoms, said method comprising including administering to the patient two 900 mg intravenous doses of an anti-IL-36R antibody; wherein the second dose is administered less than 2 weeks after the first dose.
6. A method of treating a skin disorder associated with GPP, said method comprising administering to the patient two 900 mg intravenous doses of an anti-IL-36R antibody; wherein the second dose is administered less than 2 weeks after the first dose.
7. A method of preventing the recurrence of GPP flares in a patient, said method comprising administering to the patient two 900 mg intravenous doses of an anti-IL-36R antibody; wherein the second dose is administered less than 2 weeks after the first dose.
8. A method of reducing pain by at least 10% in a patient with GPP, said method comprising administering to the patient two 900 mg intravenous doses of an anti-IL-36R antibody; wherein the second dose is administered less than 2 weeks after the first dose.
9. A method of improving the quality of life by at least 10% in a patient with moderate to severe GPP symptoms, said method comprising administering to the patient two 900 mg intravenous doses of an anti-IL-36R antibody; wherein the second dose is administered less than 2 weeks after the first dose.
10. The method according to any of the preceding clauses, wherein the patient has a GPP Physician Global Assessment (GPPGA) total score of ≥2.
11. The method according to any of the preceding clauses, wherein the patient has a GPP Physician Global Assessment (GPPGA) total score of ≥2 and a GPPGA pustulation subscore of ≥2.
12. The method according to any of the preceding clauses, wherein the patient has a GPP Physician Global Assessment (GPPGA) total score of ≥2 and a GPPGA pustulation subscore of ≥2 before the administration of the first i.v. dose.
13. The method according to any of the preceding clauses, wherein the patient has a GPP Physician Global Assessment (GPPGA) total score of ≥2 and a GPPGA pustulation subscore of ≥2 after the administration of the first i.v. dose.
14. The method according to any of the preceding clauses, wherein the patient has a GPP Physician Global Assessment (GPPGA) total score of ≥2 and a GPPGA pustulation subscore of ≥2 before and after the administration of the first i.v. dose.
15. The method according to any of the preceding clauses, wherein the second dose is administered after 1 week but less than 2 weeks from the first dose.
16. A method of treating a GPP patient with a GPPGA pustulation subscore of ≥2, said method comprising the steps of: (a) administering to the patient a first 900 mg intravenous (I.V.) dose of an anti-IL-36R antibody; (b) assessing the GPPGA pustulation subscore of the patient and if the GPPGA pustulation subscore of ≥2 of the patient persists after 1 week from the first dose, then administering to the patient a second 900 mg (i.v.) dose of spesolimab less than 2 weeks after the first dose.
17. A method of treating a GPP patient with a GPP Physician Global Assessment (GPPGA) total score of ≥2, said method comprising the steps of: (a) administering to the patient a first 900 mg intravenous (i.v.) dose of an anti-IL-36R antibody; (b) assessing the GPPGA total score of the patient and if the GPPGA total score of ≥2 of the patient persists after 1 week from the first dose, then administering to the patient a second 900 mg (i.v.) dose of spesolimab less than 2 weeks after the first dose.
18. A method of treating a GPP patient with a GPP Physician Global Assessment (GPPGA) total score of ≥2 and a GPPGA pustulation subscore of ≥2, said method comprising the steps of: (a) administering to the patient a first 900 mg intravenous (I.V.) dose of an anti-IL-36R antibody; (b) assessing the GPPGA scores of the patient and if the GPPGA total score of ≥2 and the GPPGA pustulation subscore of ≥2 of the patient persist after 1 week from the first dose, then administering to the patient a second 900 mg (i.v.) dose of spesolimab less than 2 weeks after the first dose.
19. The method according to any of the preceding clauses, wherein an optional third 900 mg i.v. dose of the anti-IL-36R antibody is administered at 2 to 12 weeks after the second i.v. dose.
20. The method according to any of the preceding clauses, wherein the two-dose administration achieves one or more of the following results:
   (a) a Generalized Pustular Psoriasis Global Assessment (GPPGA) pustulation subscore of 0 indicating in one week after administering the second i.v. dose; and/or
   (b) a GPPGA total score of 0 or 1 in one week after administering the second i.v. dose.
21. The method of clause 20, wherein the results are maintained for up to and at least 12 weeks following the administration of the second i.v. dose.
22. The method according to any of the preceding clauses, wherein the method further comprises administering to the patient a prophylactically effective amount of the anti-IL-36R antibody in one or more subcutaneous doses after the last i.v. dose administered.
23. The method according clause 22, wherein each of the one or more subcutaneous doses comprises 150 mg, 225 mg, 300 mg, 450 mg, or 600 mg of said anti-IL-36R antibody.
24. The method according to clause 23, wherein 1, 2, 3 or more subcutaneous doses are administered to the patient and wherein a first subcutaneous dose is administered after the last intravenous dose.
25. The method according to clause 24, wherein a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.
26. The method according to any of the preceding clauses, wherein the patient remains in clinical remission as measured by a GPPGA total score of 0 or 1 for at least 12, 24, 36, 48, 60 or 72 weeks following the last i.v. or subcutaneous dose.
27. The method according to any of the preceding clauses, wherein the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
28. The method according to any of the preceding clauses, wherein the anti-IL-36R antibody comprises:
   I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
   II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
   III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
   IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
   V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
   VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
29. The method according to any of the preceding clauses, wherein the anti-IL-36R antibody comprises:
   (i) a light chain variable region comprising the amino acid sequence of SEQ ID NO: 77; and a heavy chain variable region comprising the amino acid sequence of SEQ ID NO: 87; or
   (ii) a light chain variable region comprising the amino acid sequence of SEQ ID NO: 77; and a heavy chain variable region comprising the amino acid sequence of SEQ ID NO: 88; or
   (iii) a light chain variable region comprising the amino acid sequence of SEQ ID NO: 77; and a heavy chain variable region comprising the amino acid sequence of SEQ ID NO: 89; or
   (iv) a light chain variable region comprising the amino acid sequence of SEQ ID NO: 80; and a heavy chain variable region comprising the amino acid sequence of SEQ ID NO: 87; or
   (v) a light chain variable region comprising the amino acid sequence of SEQ ID NO: 80; and a heavy chain variable region comprising the amino acid sequence of SEQ ID NO: 88; or
   (vi) a light chain variable region comprising the amino acid sequence of SEQ ID NO: 80; and a heavy chain variable region comprising the amino acid sequence of SEQ ID NO: 89; or
   (vii) a light chain variable region comprising the amino acid sequence of SEQ ID NO: 85; and a heavy chain variable region comprising the amino acid sequence of SEQ ID NO: 100; or
   (viii) a light chain variable region comprising the amino acid sequence of SEQ ID NO: 85; and a heavy chain variable region comprising the amino acid sequence of SEQ ID NO:101; or
   (ix) a light chain variable region comprising the amino acid sequence of SEQ ID NO: 86; and a heavy chain variable region comprising the amino acid sequence of SEQ ID NO: 100; or
   (x) a light chain variable region comprising the amino acid sequence of SEQ ID NO: 86; and a heavy chain variable region comprising the amino acid sequence of SEQ ID NO:101.
30. The method according to any of the preceding clauses, wherein the anti-IL-36R antibody comprises:
   i. a light chain comprising the amino acid sequence of SEQ ID NO: 115; and a heavy chain comprising the amino acid sequence of SEQ ID NO: 125; or
   ii. a light chain comprising the amino acid sequence of SEQ ID NO: 115; and a heavy chain comprising the amino acid sequence of SEQ ID NO: 126; or
   iii. a light chain comprising the amino acid sequence of SEQ ID NO: 115; and a heavy chain comprising the amino acid sequence of SEQ ID NO: 127; or
   iv. a light chain comprising the amino acid sequence of SEQ ID NO: 118; and a heavy chain comprising the amino acid sequence of SEQ ID NO: 125; or
   v. a light chain comprising the amino acid sequence of SEQ ID NO: 118; and a heavy chain comprising the amino acid sequence of SEQ ID NO: 126; or
   vi. a light chain comprising the amino acid sequence of SEQ ID NO: 118; and a heavy chain comprising the amino acid sequence of SEQ ID NO: 127; or
   vii. a light chain comprising the amino acid sequence of SEQ ID NO: 123; and a heavy chain comprising the amino acid sequence of SEQ ID NO: 138; or
   viii. a light chain comprising the amino acid sequence of SEQ ID NO: 123; and a heavy chain comprising the amino acid sequence of SEQ ID NO: 139; or
   ix. a light chain comprising the amino acid sequence of SEQ ID NO: 124; and a heavy chain comprising the amino acid sequence of SEQ ID NO: 138.
31. The method according to any of the preceding clauses, wherein the anti-IL-36R antibody is spesolimab.
32. A method of treating generalized pustular psoriasis (GPP) flares in a patient, said method comprising administering to the patient two intravenous (i.v.) doses of 900 mg spesolimab; wherein the second dose is administered less than 2 weeks after the first dose.
33. A method of treating GPP in a patient, said method comprising administering to the patient two intravenous doses of 900 mg spesolimab; wherein the second dose is administered less than 2 weeks after the first dose.
34. A method of reducing or alleviating signs and symptoms of an acute phase flare-up of GPP in a patient, said method comprising administering to the patient two intravenous doses of 900 mg spesolimab; wherein the second dose is administered less than 2 weeks after the first dose.
35. A method of reducing the severity and duration of GPP symptoms, said method comprising including administering to the patient two intravenous doses of 900 mg spesolimab; wherein the second dose is administered less than 2 weeks after the first dose.
36. A method of treating a skin disorder associated with GPP, said method comprising administering to the patient two intravenous doses of 900 mg spesolimab; wherein the second dose is administered less than 2 weeks after the first dose.
37. A method of preventing the recurrence of GPP flares in a patient, said method comprising administering to the patient two intravenous doses of 900 mg spesolimab; wherein the second dose is administered less than 2 weeks after the first dose.
38. A method of reducing pain by at least 10% in a patient with GPP, said method comprising administering to the patient two intravenous doses of 900 mg spesolimab; wherein the second dose is administered less than 2 weeks after the first dose.
39. A method of improving the quality of life by at least 10% in a patient with moderate to severe GPP symptoms, said method comprising administering to the patient two intravenous doses of 900 mg spesolimab; wherein the second dose is administered less than 2 weeks after the first dose.
40. The method according to any of the preceding clauses, wherein the patient has a GPP Physician Global Assessment (GPPGA) total score of ≥2.
41. The method according to any of the preceding clauses, wherein the patient has a GPP Physician Global Assessment (GPPGA) total score of ≥2 and a GPPGA pustulation subscore of ≥2.
42. The method according to any of the preceding clauses, wherein the patient has a GPP Physician Global Assessment (GPPGA) total score of ≥2 and a GPPGA pustulation subscore of ≥2 before the administration of the first i.v. dose.
43. The method according to any of the preceding clauses, wherein the patient has a GPP Physician Global Assessment (GPPGA) total score of ≥2 and a GPPGA pustulation subscore of ≥2 after the administration of the first i.v. dose.
44. The method according to any of the preceding clauses, wherein the patient has a GPP Physician Global Assessment (GPPGA) total score of ≥2 and a GPPGA pustulation subscore of ≥2 before and after the administration of the first i.v. dose.
45. The method according to any of the preceding clauses, wherein the second dose is administered after 1 week but less than 2 weeks from the first dose.
46. A method of treating a GPP patient with a GPPGA pustulation subscore of ≥2, said method comprising the steps of: (a) administering to the patient a first intravenous (i.v.) dose of 900 mg spesolimab; (b) assessing the GPPGA pustulation subscore of the patient and if the GPPGA pustulation subscore of ≥2 of the patient persists after 1 week from the first dose, then administering to the patient a second (i.v.) dose of 900 mg spesolimab less than 2 weeks after the first dose.
47. A method of treating a GPP patient with a GPP Physician Global Assessment (GPPGA) total score of ≥2, said method comprising the steps of: (a) administering to the patient a first intravenous (i.v.) dose of 900 mg spesolimab; (b) assessing the GPPGA total score of the patient and if the GPPGA total score of ≥2 of the patient persists after 1 week from the first dose, then administering to the patient a second (i.v.) dose of 900 mg spesolimab less than 2 weeks after the first dose.
48. A method of treating a GPP patient with a GPP Physician Global Assessment (GPPGA) total score of ≥2 and a GPPGA pustulation subscore of ≥2, said method comprising the steps of: (a) administering to the patient a first intravenous (I.V.) dose of 900 mg spesolimab; (b) assessing the GPPGA scores of the patient and if the GPPGA total score of ≥2 and the GPPGA pustulation subscore of ≥2 of the patient persist after 1 week from the first dose, then administering to the patient a second (i.v.) dose of 900 mg spesolimab less than 2 weeks after the first dose.
49. The method according to any of the preceding clauses, wherein an optional third i.v. dose of 900 mg spesolimab is administered at 2 to 12 weeks after the second i.v. dose.
50. The method according to any of the preceding clauses, wherein the two-dose administration achieves one or more of the following results:
   (a) a Generalized Pustular Psoriasis Global Assessment (GPPGA) pustulation subscore of 0 indicating in one week after administering the second i.v. dose; and/or
   (b) a GPPGA total score of 0 or 1 in one week after administering the second i.v. dose.
51. The method of clause 50, wherein the results are maintained for up to and at least 12 weeks following the administration of the second i.v. dose.
52. The method according to any of the preceding clauses, wherein the method further comprises administering to the patient a prophylactically effective amount of spesolimab in one or more subcutaneous doses after the last i.v. dose administered.
53. The method according clause 52, wherein each of the one or more subcutaneous doses comprises 150 mg, 225 mg, 300 mg, 450 mg, or 600 mg spesolimab.
54. The method according to clause 53, wherein 1, 2, 3 or more subcutaneous doses are administered to the patient and wherein a first subcutaneous dose is administered after the last intravenous dose.
55. The method according to clause 54, wherein a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.
56. The method according to any of the preceding clauses, wherein the patient remains in clinical remission as measured by a GPPGA total score of 0 or 1 for at least 12, 24, 36, 48, 60 or 72 weeks following the last i.v. or subcutaneous dose.
57. The method according to any of the preceding clauses, wherein the patient experiences an improvement in Psoriasis Symptom Scale (PSS) score within 1, 4 or 12 weeks after receiving the dose(s) of spesolimab; wherein the improvement is at least 10% when the PSS score after the anti-IL-36R antibody or spesolimab administration is compared to the PSS score at baseline in the patient before the administration.
58. The method according to any of the preceding clauses, wherein the patient experiences an improvement in Functional Assessment of Chronic Illness Therapy-Fatigue (FACIT-Fatigue) score within 1, 4 or 12 weeks after receiving the dose(s) of spesolimab; wherein the improvement is at least 10% when the FACIT-Fatigue score after the anti-IL-36R antibody or spesolimab administration is compared to the FACIT-Fatigue score at baseline in the patient before the administration.
59. The method according to any of the preceding clauses, wherein the patient experiences an improvement in Dermatology Quality of Life Index (DLQI), Psoriasis System Scale (PSS), Visual Analogue Scale (VAS) or Functional Assessment of Chronic Illness Therapy-Fatigue (FACIT-Fatigue) score within 1, 4 or 12 weeks after receiving the dose(s) of spesolimab; wherein the improvement is at least 10% when the DQLI, PSS, VAS or FACIT-Fatigue score after the anti-IL-36R antibody or spesolimab administration at week 1, 4 or 12 is compared to the DQLI, PSS, VAS or FACIT-Fatigue score at baseline in the patient before the administration.
60. A method of improving Psoriasis Symptom Scale (PSS), Dermatology Quality of Life Index (DLQI), Visual Analogue Scale (VAS) or Functional Assessment of Chronic Illness Therapy-Fatigue (FACIT-Fatigue) score in a patient with GPP, said method comprising administering to the patient one 900 mg intravenous (i.v.) dose of spesolimab and optional second 900 mg i.v. dose of spesolimab; wherein the second optional dose of spesolimab is administered less than 2 weeks after the first dose if the GPP symptoms (e.g., a GPPGA total score of ≥2 and/or a GPPGA pustulation subscore of ≥2) in the patient persist.
61. A method of improving Functional Assessment of Chronic Illness Therapy-Fatigue (FACIT-Fatigue) score in a patient with GPP, said method comprising administering to the patient one 900 mg intravenous (i.v.) dose of spesolimab and optional second 900 mg i.v. dose of spesolimab; wherein the second optional dose of spesolimab is administered less than 2 weeks after the first dose if the GPP symptoms (e.g., a GPPGA total score of ≥2 and/or a GPPGA pustulation subscore of ≥2) in the patient persist.
62. A method of improving Dermatology Quality of Life Index (DLQI) score in a patient with GPP, said method comprising administering to the patient one 900 mg intravenous (i.v.) dose of spesolimab and optional second 900 mg i.v. dose of spesolimab; wherein the second optional dose of spesolimab is administered less than 2 weeks after the first dose if the GPP symptoms (e.g., a GPPGA total score of ≥2 and/or a GPPGA pustulation subscore of ≥2) in the patient persist.
63. The method according to any of clauses 59 to 61, wherein the improvement is at least 10% when the score after the spesolimab administration at week 1, 4 or 12 is compared to the score at baseline in the patient before the administration.
64. The method according to any of the preceding clauses, wherein the spesolimab administration(s) result(s) in a lower maximum ADA titers at week(s) 2, 4, 6, 8, 10 and/or 12 of the treatment.

Further, the pharmaceutical composition can be provided as a pharmaceutical kit comprising (a) a container containing a IL-36R binding agent (e.g., an anti-IL-36R antibody) in lyophilized form and (b) a second container containing a pharmaceutically acceptable diluent (e.g., sterile water) for injection. The pharmaceutically acceptable diluent can be used for reconstitution or dilution of the lyophilized anti-IL-36R antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an anti-IL-36R antibody or IL-36R binding agent is administered concurrently with a therapeutic agent. In another specific embodiment, the therapeutic agent is administered prior or subsequent to administration of the anti-IL-36R antibody or IL-36R binding agent, by at least an hour and up to several months, for example at least an hour, five hours, 12 hours, a day, a week, a month, or three months, prior or subsequent to administration of the anti-IL-36R antibody or IL-36R binding agent.

The invention is further described in the following examples, which are not intended to limit the scope of the invention.

### EXAMPLES

### Example 1: Trial of Spesolimab for Generalized Pustular Psoriasis

### Abstract

Background: Generalized pustular psoriasis is a rare, life-threatening, inflammatory skin disease characterized by widespread eruption of sterile pustules. Dysregulated interleukin-36 signaling is involved in its pathogenesis. We compared spesolimab, a humanized anti-interleukin-36 receptor monoclonal antibody with placebo in a phase 2 trial in patients with flares of generalized pustular psoriasis.

Methods: Patients with a flare of generalized pustular psoriasis were randomly assigned in a 2:1 ratio to a single 900 mg intravenous dose of spesolimab or placebo. The primary endpoint was a Generalized Pustular Psoriasis Physician Global Assessment (GPPGA) pustulation subscore of 0 (range 0 to 4, 0 indicating no pustules and 4 indicating severe pustulation) at week 1. The key secondary endpoint was a GPPGA total score of 0 or 1 (clear or almost clear) at week 1 (range 0 to 4, higher scores indicating greater disease severity).

Results: A total of 85 patients were screened and 53 were enrolled: 35 were assigned to receive spesolimab and 18 to receive placebo. Baseline GPPGA pustulation subscores were 3 in 46% and 39%, and 4 in 37% and 33%, of the spesolimab and placebo groups, respectively. At the end of week 1, a pustulation subscore of 0 was achieved in 19/35 (54%) of patients receiving spesolimab versus 1/18 (6%) receiving placebo (difference, 49 percentage points; 95% confidence interval [CI] 21.5 to 67.2]; P<0.001). At the end of week 1, a GPPGA total score of 0 or 1 was achieved by 15/35 (43%) of patients in the spesolimab group and by 2/18 (11%) of patients in the placebo group (difference, 32 percentage points; 95% CI, 2.2 to 52.7]; P=0.024). Drug reactions with eosinophilia and systemic symptoms were reported in two spesolimab-treated patients. Infections occurred in 17% of spesolimab-treated patients at week 1. Anti-drug antibodies were detected in 23/50 (46%) patients who received at least one dose of spesolimab.

Conclusions: In a 1 week phase 2 trial in patients with a flare of generalized pustular psoriasis, the IL-36 receptor inhibitor spesolimab, resulted in higher rates of lesion clearance than placebo but was associated with more infections and systemic drug reactions. Longer and larger trials are required to determine the effect and risks of spesolimab in pustular psoriasis. (Funded by Boehringer Ingelheim; ClinicalTrials.gov identifier: NCT03782792).

### Introduction

Generalized pustular psoriasis (GPP) is a rare, potentially life-threatening, autoinflammatory skin disease characterized by widespread eruption of sterile, visible pustules that occurs with or without systemic symptoms of pain, fever, general malaise, fatigue, and extra cutaneous manifestations such as arthritis and neutrophilic cholangitis. The clinical course of GPP can be relapsing with recurrent flares, or persistent with intermittent flares. Mortality rates range from 2% to 16% and have been attributed to septic shock and cardiorespiratory failure. The frequency of flares varies among patients, and flares may be spontaneous or triggered by upper respiratory tract infections, stress, medication, medication withdrawal and pregnancy. The disease has an adverse impact on quality of life.

There are no approved therapies for the disease in the United States or

Europe and management has included cyclosporine, retinoids, methotrexate and biologics. Biologic agents that inhibit tumor necrosis factor-alpha (adalimumab, infliximab, certolizumab pegol), interleukin (IL)-17/IL-17 receptor (secukinumab, brodalumab, ixekizumab), and IL-23 (risankizumab, guselkumab) are approved for use in Japan, Taiwan and Thailand based on small trials and studies of these drugs in plaque psoriasis and small non-randomized trials in GPP.

The role of the IL-36 pathway in GPP is supported by the finding of loss-of-function mutations in the IL-36 receptor antagonist gene (IL36RN), and associated genes (CARD14, AP1S3, SERPINA3, MPO), and the overexpression of IL-36 cytokines in GPP skin lesions. Clinical improvements with spesolimab, a humanized anti-IL-36 receptor monoclonal antibody, were observed in an open-label phase 1 study in seven patients presenting with a GPP flare.

We conducted a phase 2 randomized trial to investigate the efficacy and safety of spesolimab versus placebo in patients presenting with a GPP flare. Because acute and severe flares of this disorder are life-threatening, a single dose of the drug with a placebo-controlled period of 1 week was chosen for the trial design and patients from both arms were offered the opportunity to receive open-label spesolimab on day 8. At week 12, the end of the trial, patients were offered enrolment into a separate open-label extension trial of spesolimab (ClinicalTrials.gov identifier: NCT03886246).

### Methods

### Trial Design

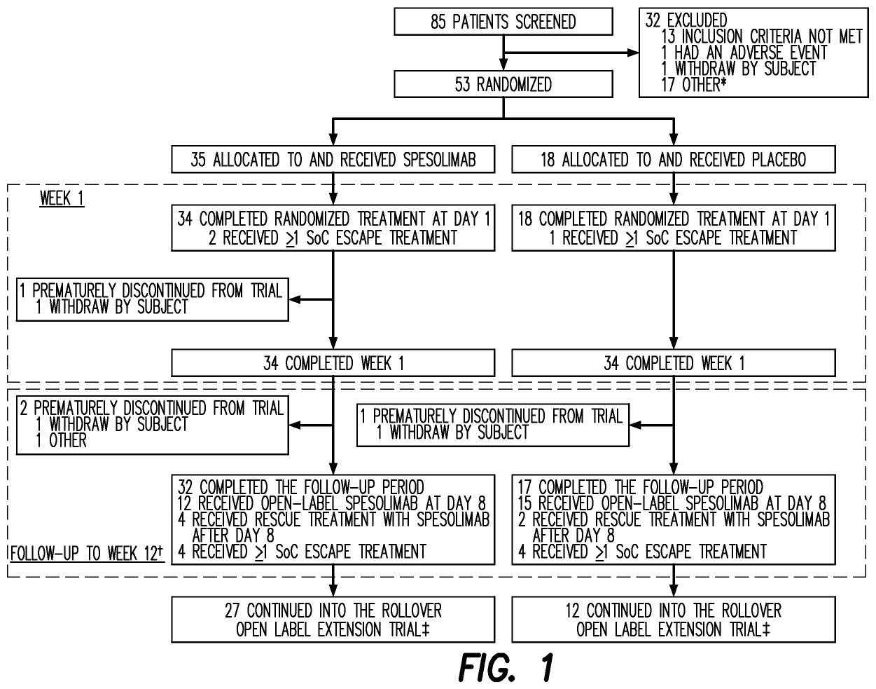

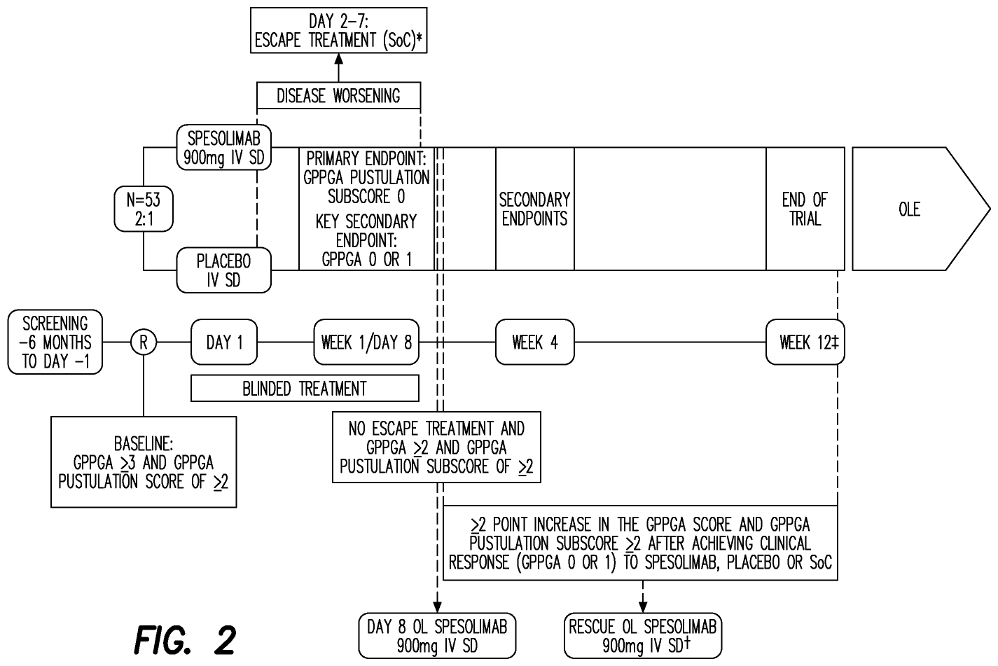

This phase 2, multicenter, randomized, double-blind, placebo-controlled trial was conducted between 20 Feb. 2019 and 5 Jan. 2021, and enrolled patients from 37 sites in 12 countries. Patients presenting with a GPP flare were randomly assigned in a 2:1 ratio to receive a single intravenous dose of 900 mg spesolimab or placebo (FIG. 1). Randomization was performed using an interactive response system with stratification factor of Japanese race versus non-Japanese race. Patients and investigators were masked to treatment group assignment administered on day 1 throughout the trial until the database was locked for analyses. On day 8, patients from both arms were eligible to receive an open-label, single, intravenous dose of 900 mg spesolimab (which led to a cross-over from placebo to spesolimab for some patients) if they had persistent symptoms based on a pre-defined threshold that consisted of a GPP Physician Global Assessment (GPPGA) total score at the end of week 1 (range from 0 [clear skin] to 4 [severe disease]) and a clinician assessment of GPP severity based on a modified PGA and a GPPGA pustulation subscore at week 1 (range from 0 [no visible pustules] to 4 [severe pustulation]. The GPPGA total score is the average of the sub-scores [pustulation, erythema, and scaling]; (see the Supplementary information below). After week 1, rescue treatment with a single intravenous dose of 900 mg spesolimab could be administered in case of reoccurrence of a flare (defined as point increase in both the GPPGA total score and the pustulation subscore after first attaining a GPPGA total score of 0 or 1). Patients who achieved clinical improvement and completed the trial without flare symptoms were eligible to enter a 5-year open-label extension trial (ClinicalTrials.gov identifier: NCT03886246). Escape treatment with standard of care, per the treating physician's choice, was allowed for patients who experienced worsening of disease that required immediate treatment during week 1, and for patients with disease worsening who did not qualify for a rescue medication with open-label spesolimab after week 1. The use of escape treatment in the first week was essential for patient safety because flares of GPP are potentially life threatening. Any patient who received escape medication was considered not achieving a response (non response) in the analysis for the primary and key secondary evaluation at week 1. Details of the trial design are in the protocol described below and in FIG. 2.

Patients aged 18-75 years were eligible for enrollment if they had a history of GPP consistent with criteria for diagnosis according to European Rare And Severe Psoriasis Expert Network (ERASPEN) criteria 1. Analyses of coding sequences for the three main GPP-associated genes (IL36RN, CARD14, AP1S3) were performed on DNA extracts from blood samples and patients were enrolled without regard to IL36RN mutation status. Patients had to have a GPP flare of moderate-to-severe intensity (defined as total GPPGA score ≥3, new or worsening pustules, a GPPGA pustulation subscore ≥2, and ≥5% body surface area with erythema and the presence of pustules). Key exclusion criteria included patients with plaque psoriasis without pustules or with pustules restricted to psoriatic plaques, drug-triggered acute generalized exanthematous pustulosis, immediate life-threatening flare of GPP requiring intensive care treatment, and requirement for current treatment with methotrexate, cyclosporine, or retinoids, or any restricted medication.

### Endpoints

The primary endpoint was the achievement of a GPPGA pustulation subscore of 0 (clear) at the end of week 1. The key secondary endpoint was a GPPGA score of 0 or 1 (clear or almost clear) at the end of week 1. Secondary endpoints were primarily at week 4, when some patients might have received open-label spesolimab on day 8. Secondary endpoints at week 4 included: a 75% or greater improvement in Psoriasis Area and Severity Index for Generalized Pustular Psoriasis (GPPASI 75; GPPASI is an adaptation of the PASI score in which induration is replaced by a pustule component; scores range from 0 [least severe] to 72 [most severe], change from baseline in visual analog pain scale (VAS, range from 0 [no pain] to 100 [severe pain]), change from baseline in Psoriasis Symptom Scale (PSS; patient-reported psoriasis pain, redness, itching, and burning; range from 0 to 16, with higher scores indicating more severe symptoms), and change from baseline in Functional Assessment of Chronic Illness Therapy-Fatigue (FACIT-Fatigue; patient-reported impact of fatigue on daily activities; range from 0 to 52, with lower scores indicating greater impact). There were two additional secondary endpoints assessed at week 1 and/or 4 as listed below and the protocol and statistical analysis plan.

Details of trial outcomes, as well as a list of exploratory endpoints are listed in Supplementary Table S3 below.

Safety events at week 1 (through the first 8 days of the study) and through week 12 included treatment-emergent and serious adverse events. Adverse events were assessed by the trial investigators, who were masked to treatment assignments until after the database lock for the final analysis of the trial. During the course of the trial, adverse events occurring were collected, documented on the electronic case reports, and reported to the sponsor by the investigator. The intensity of the adverse events was assessed by the investigators and graded according to RCTC Version 2.0 developed by the Outcome Measures in Rheumatology (OMERACT) organization.

### Statistical Analysis

A sample size of 51 patients was estimated to provide ≥90% power to detect any difference between spesolimab and placebo with assumed response rates of 0.6 and 0.1, respectively, for both the primary and key secondary endpoints, and a type I error of <0.025 (one-sided), which can be considered as a type I error of <0.05 with a two-sided test. The primary endpoint and key secondary endpoint were analyzed using the randomized set with an exact Suissa-Shuster Z-pooled test. This is a one-sided test; two-sided P-value was reported by doubling the one-sided P-value. 30 Confidence intervals (95% CI) around the risk difference were calculated using the Chan and Zhang method for the primary and all binary secondary endpoints. The fixed sequence test was used to control the familywise type I error. The primary and key secondary endpoints (both assessed at day 8 [week 1]) were tested in a hierarchical manner at a two-sided level of P<0.05. If the primary endpoint failed to reach a significant difference between the trial groups, the key secondary outcome would not be tested. The protocol and statistical analysis plan called for hierarchical testing of 4 subsequent secondary endpoints (GPPASI 75 and change from baseline in: pain VAS; PSS; and FACIT-Fatigue), all at week 4; however, randomization to trial groups no longer pertained after week 1 as 15 of 18 patients assigned to placebo received open-label spesolimab on day 8 and were imputed with non-response or the worst possible outcome. Therefore, comparisons by randomized treatment as originally planned were non-informative and the changes at week 1 and 4 for these endpoints are reported descriptively by the following groups: All patients randomized to spesolimab (patients who received one or two doses), patients randomized to a single dose of spesolimab only (patients received spesolimab on day 1 only), patients randomized to spesolimab who received open-label day 8 spesolimab (Patients who qualified for a second dose on day 8), and patients randomized to placebo who received open-label spesolimab on day 8 (patients who received one dose of spesolimab on day 8). For Binary outcomes, patients with missing data were considered not to have reached the respective endpoint. For continuous outcomes, last observed case carried forward was used for imputation.

Post hoc sensitivity analyses of the primary and key secondary endpoints using linear regression with adjustment for the imbalanced covariates at baseline, including gender, race and GPPASI were performed but no conclusions can be drawn from these data.

### Results

### Patients

Of 85 patients screened, 53 were enrolled: 35 assigned to 900 mg spesolimab and 18 to placebo (FIG. 1; Table 1). Baseline demographic and disease characteristics differed between arms in respect to the percentage of female participants (60.0% and 83.3%, in the spesolimab and placebo arms, respectively) and the proportion of Asian participants (45.7% and 72.2%, respectively). Baseline median GPPASI total score was 27.4 in the spesolimab arm and 20.9 in the placebo arm (Table 1). At the time of randomization, 18.9% of patients had a GPPGA total score of 4 (severe), and most patients had a GPPGA pustulation subscore of 3 or 4 (high or very high density of pustules) and impaired quality of life and clinical burden as indicated by Dermatology Life Quality Index (DLQI), pain VAS, FACIT-Fatigue, and PSS (Supplementary Table S3). Seven patients, five in the spesolimab arm and two in the placebo arm, had IL36RN mutations (Table 1). Most patients had no CARD14 (38 patients without) or AP1S3 (42 without) mutations (Supplementary Table S4).

TABLE 1
Demographics and Baseline Characteristics for Current Flare

| | Spesolimab | | Placebo | |
|---|---|---|---|---|
| Characteristic | (N = 35) | | (N = 18) | |
| Mean age (SD) - yr | 43.2 | (12.1) | 42.6 | (8.4) |
| Mean weight (SD) - kg | 73.7 | (24.0) | 68.8 | (26.6) |
| Female - no. (%) | 21 | (60.0) | 15 | (83.3) |
| Asian race* - no. (%) | 16 | (45.7) | 13 | (72.2) |
| White race* - no. (%) | 19 | (54.3) | 5 | (27.8) |

GPPGA total score^{†}

| | | | | |
|---|---|---|---|---|
| 3 - no. (%) | 28 | (80.0) | 15 | (83.3) |
| 4 - no. (%) | 7 | (20.0) | 3 | (16.7) |

GPPGA pustulation subscore^{‡}

| | | | | |
|---|---|---|---|---|
| 2 - no. (%) | 6 | (17.1) | 5 | (27.8) |
| 3 - no. (%) | 16 | (45.7) | 7 | (38.9) |
| 4 - no. (%) | 13 | (37.1) | 6 | (33.3) |
| Median GPPASI total | 27.4 | (15.5-36.8) | 20.9 | (12.0-32.0) |
| score (IQR)^{§} | | | | |

IL36RN mutation^{¶}

| | | | | |
|---|---|---|---|---|
| Yes - no. (%) | 5 | (14.3) | 2 | (11.1) |
| No - no. (%) | 24 | (68.6) | 12 | (66.7) |

*Race was reported by the patient.
^{†}Generalized Pustular Psoriasis Physician Global Assessment (GPPGA) scores range from 0 (clear skin) to 4 (severe disease).
^{‡}GPPGA pustulation subscores range from 0 (no visible pustules) to 4 (very high-density pustules with pustular lakes).
^{§}Psoriasis Area and Severity Index for Generalized Pustular Psoriasis (GPPASI) scores range from 0 (least severe) to 72 (most severe).
^{¶}Five patients had homozygous mutations (four p.Leu27Pro; one p.Ser113Leu), and two patients had heterozygous mutations (p.Ser113Leu; p.Ser113Leu/p.Val44Met). At the date of database lock (18 Jan. 2021), DNA sequencing (targeted re-sequencing Illumina Miseq, Illumina Inc., San Diego, CA) was not yet completed in three patients and there were missing samples from seven patients.
IQR, interquartile range;
SD, standard deviation.

A total of 52 of the 53 enrolled patients completed the first week of the trial. Data for one patient in the spesolimab arm was missing for primary and key secondary endpoints and was imputed to no response. At day 8, 12 patients (34.3%) in the spesolimab group and 15 patients (83.3%) in the placebo group received an open-label dose of spesolimab. After day 8, 32 patients (91.4%) initially randomized to spesolimab and 17 patients (94.4%) initially randomized to placebo completed the 12-week follow-up period, during which four and two patients, respectively, required rescue treatment with spesolimab. After completing 12 weeks of treatment, 39 patients enrolled into the open-label extension trial (FIG. 1).

### Efficacy

### Primary and Key Secondary Efficacy Endpoints

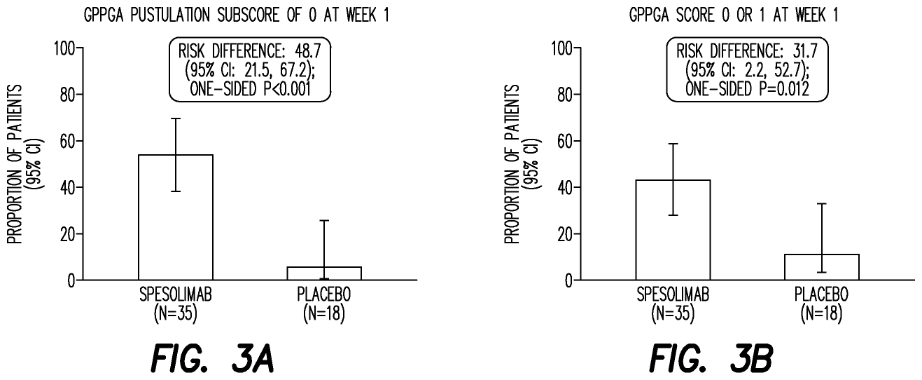

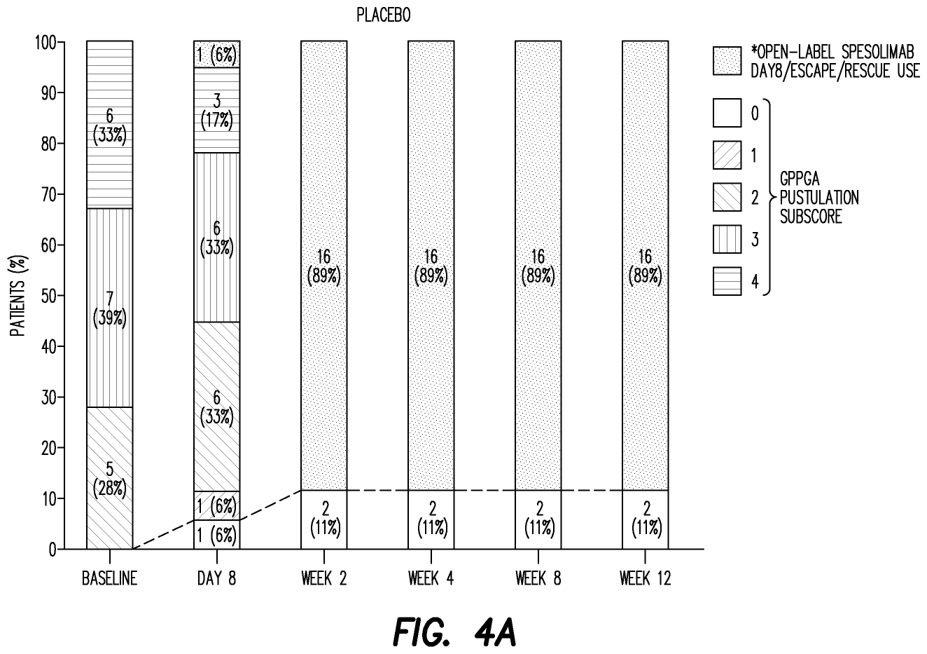

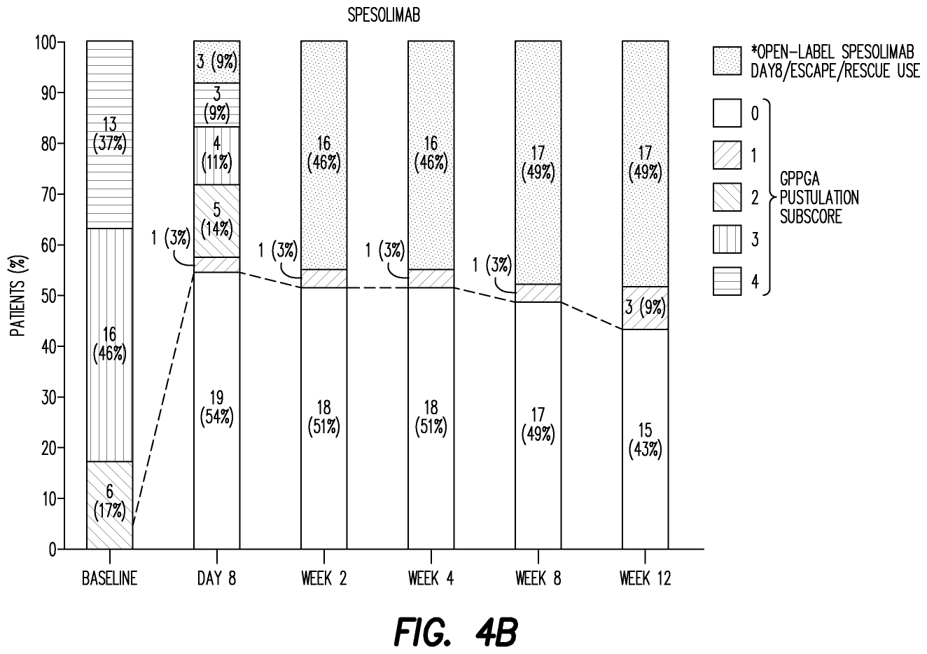

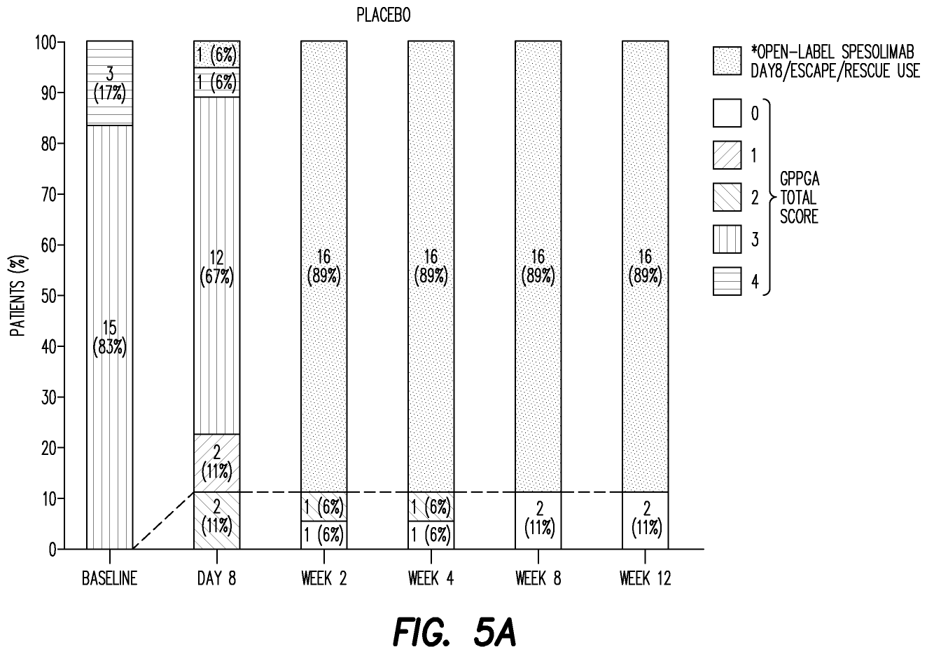

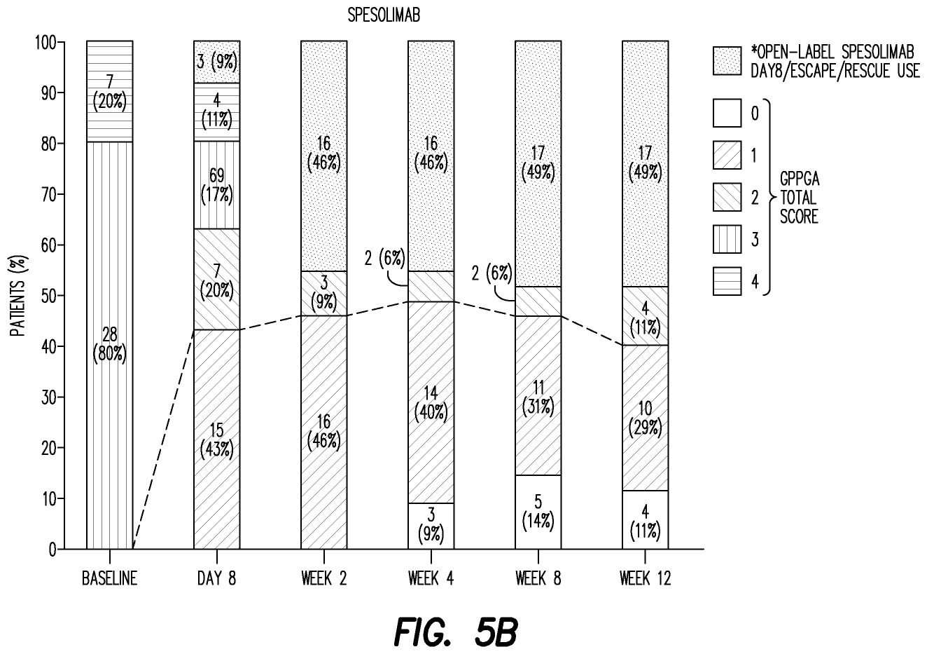

At the end of week 1, 19 patients (54.3%) randomized to spesolimab versus one patient (5.6%) randomized to placebo achieved a GPPGA pustulation subscore of 0 (no visible pustules); (difference, 48.7 percentage points; 95% confidence interval [CI], 21.5 to 67.2; P<0.001; Table 2, FIG. 3A). A GPPGA total score of 0 or 1 (clear or almost clear skin) at week 1 was achieved in 15 patients (42.9%) randomized to spesolimab versus two patients (11.1%) randomized to placebo (difference, 31.7 percentage points; 95% CI, 2.2 to 52.7; P=0.024; Table 2, FIG. 3B). The GPPGA pustulation subscores and the GPPGA total scores over time only considering randomized treatment at day 1 are shown in FIGS. 4A and 4B and FIGS. 5A and 5B.

TABLE 2
Primary and Key Secondary Efficacy Endpoints

| | Spesolimab | Placebo |
|---|---|---|
| | (N = 35) | (N = 18) |
| Primary endpoint | | |
| GPPGA pustulation subscore of 0 at week 1 | | |
| Responders - no. (%) | 19 (54.3) | 1 (5.6) |
| Difference vs placebo | 48.7 percentage points | |
| (95% CI) | (21.5 to 67.2) | |
| P-value* | <0.001 | |
| Key secondary endpoint | | |
| GPPGA total score of 0 or 1 at week 1 | | |
| Responders - no. (%) | 15 (42.9) | 2 (11.1) |

| | |
|---|---|
| Difference vs placebo | 31.7 percentage points |
| (95% CI) | (2.2 to 52.7) |
| P-value* | 0.024 |

P-value* 0.024
*By Suissa-Shuster Z-pooled test, two-sided P-value.
CI, confidence interval;
GPPGA, Generalized Pustular Psoriasis Physician Global Assessment.

Post-hoc sensitivity analyses of the primary and key secondary endpoints to adjust for the observed baseline imbalance in gender, race, and GPPASI were consistent with the primary analysis. (Supplementary Tables S5 and S6).

### Exploratory Efficacy Endpoints Post Day 8 by Randomized Treatment at Day 1 and Open-Label Spesolimab Treatment at Day 8

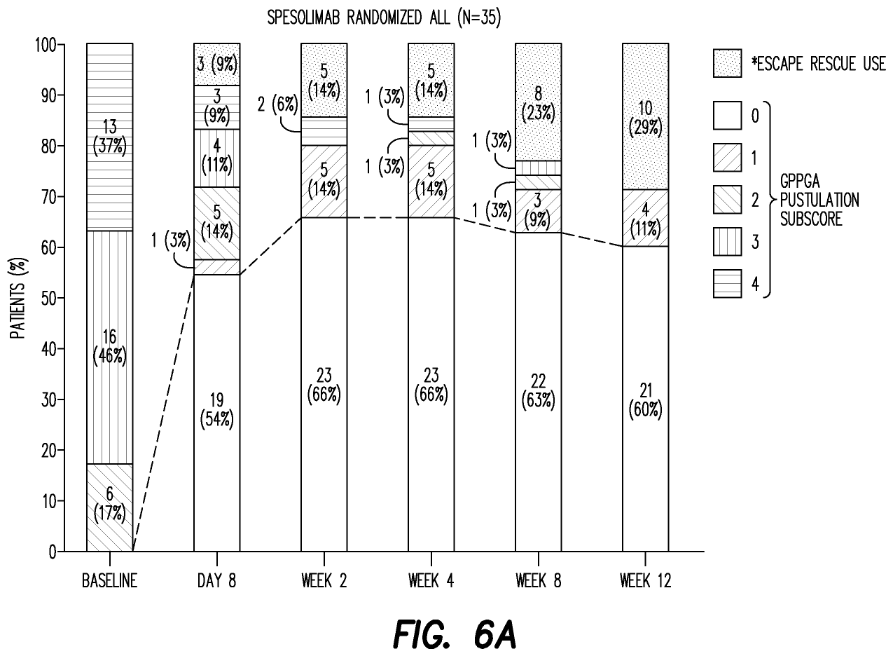

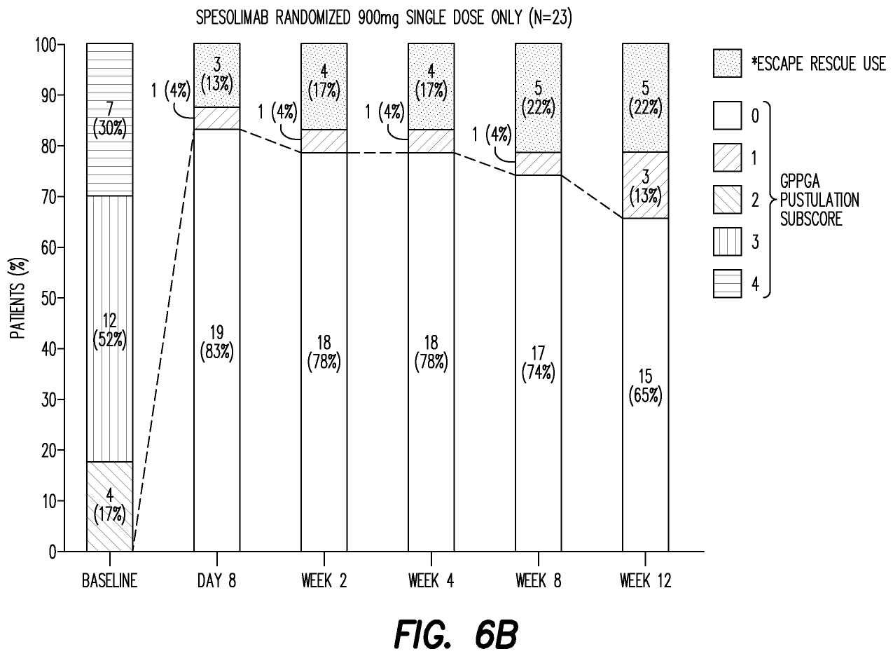

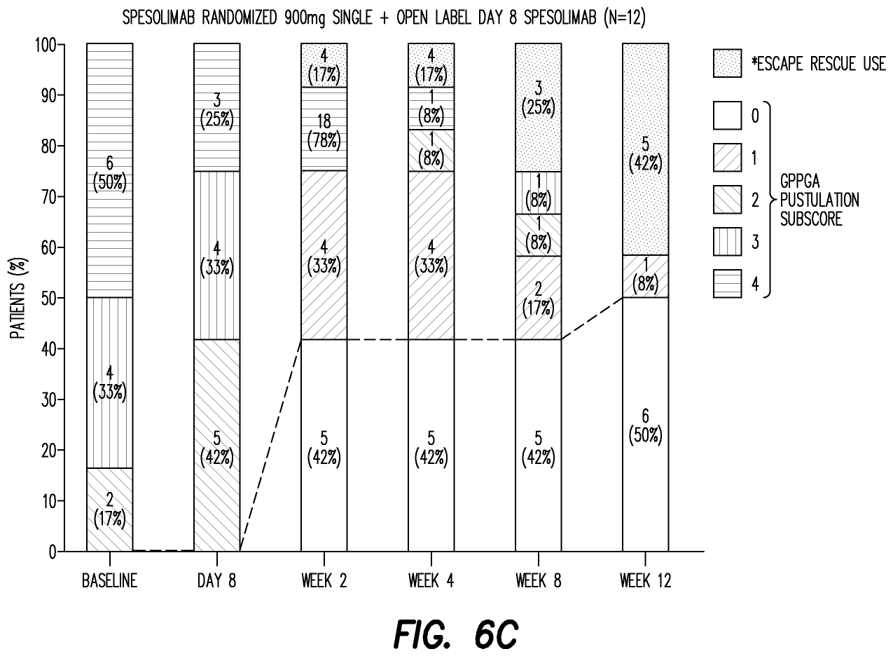

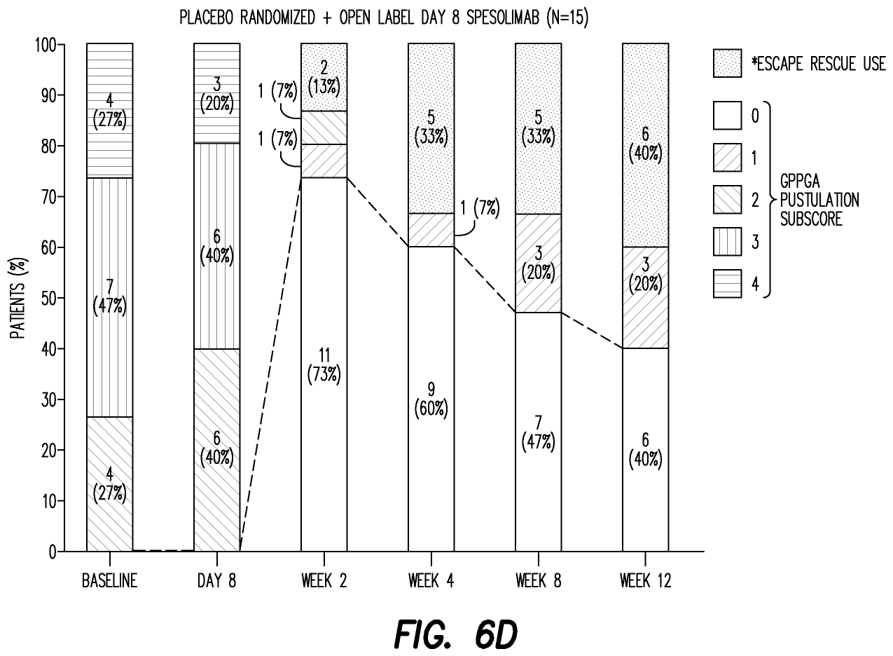

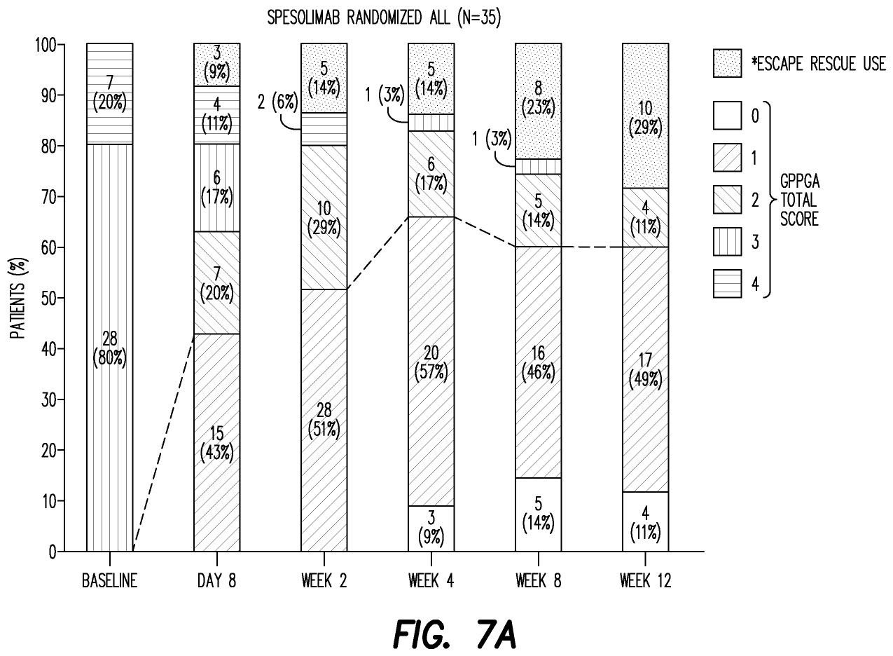

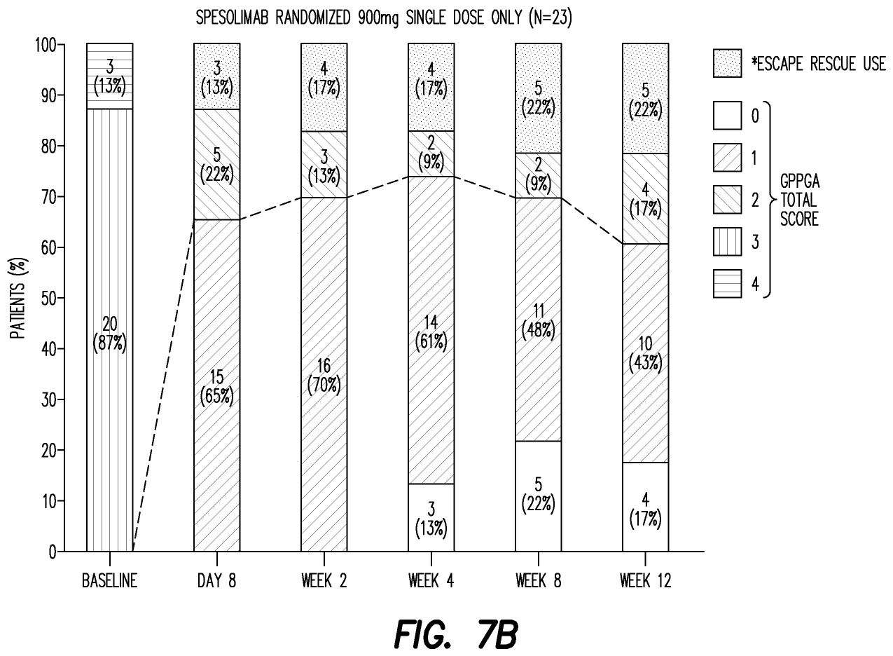

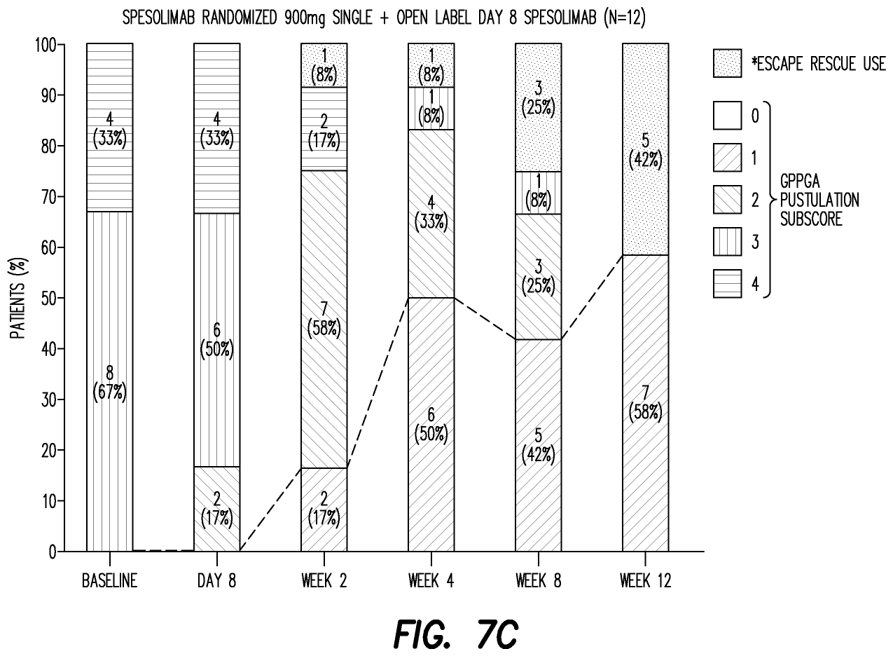

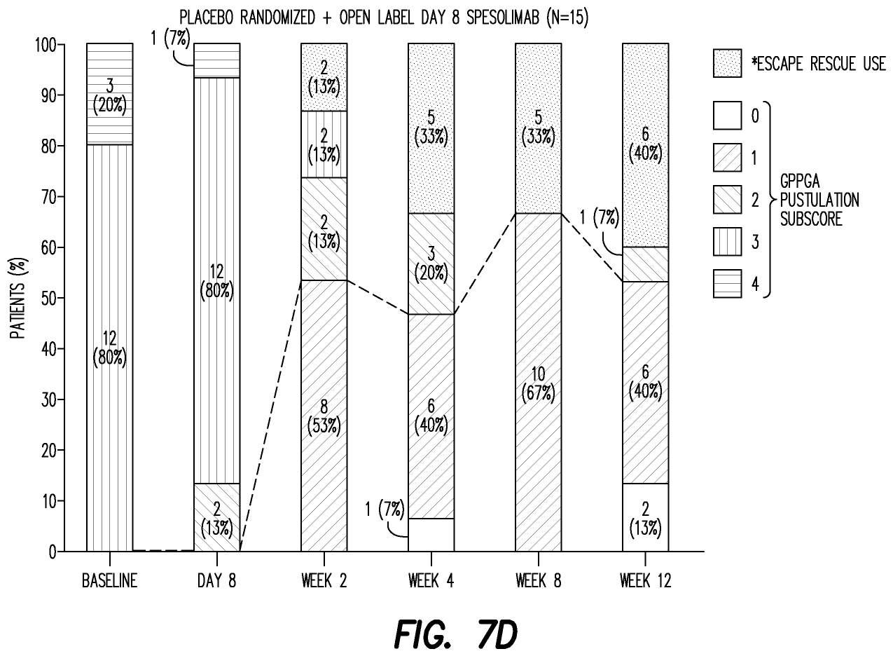

After week 1, 15/18 patients assigned to placebo received open-label spesolimab on day 8, thus planned hierarchical testing of comparative secondary endpoints at week 4 were non-informative. Instead, the secondary endpoints were reported descriptively by four groups that reflected the treatment paths post day 8: All patients randomized to spesolimab (patients who received one [day 1 only] or two doses [day 1 plus day 8]; N=35), patients randomized to a single dose of spesolimab only (day 1; N=23), patients randomized to spesolimab who received open-label day 8 spesolimab (day 1 plus day 8; N=12), and patients randomized to placebo who received open-label spesolimab on day 8 (N=15). Descriptive analyses for GPPGA pustulation subscore of 0 and GPPGA total score of 0 or 1 for these subgroups over time are reported in the Supplementary information (below) and FIGS. 6A-6D and FIGS. 7A-7D and secondary endpoints including GPPASI 75 and change from baseline in: pain VAS, PSS, and FACIT-Fatigue at week 4 are reported in the Supplementary information below.

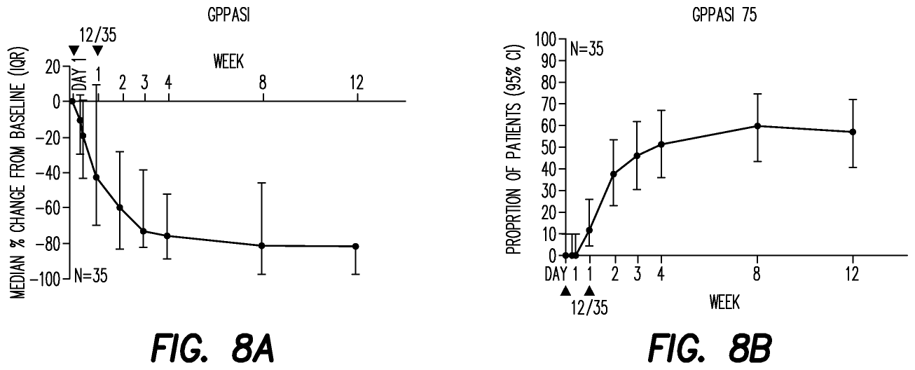

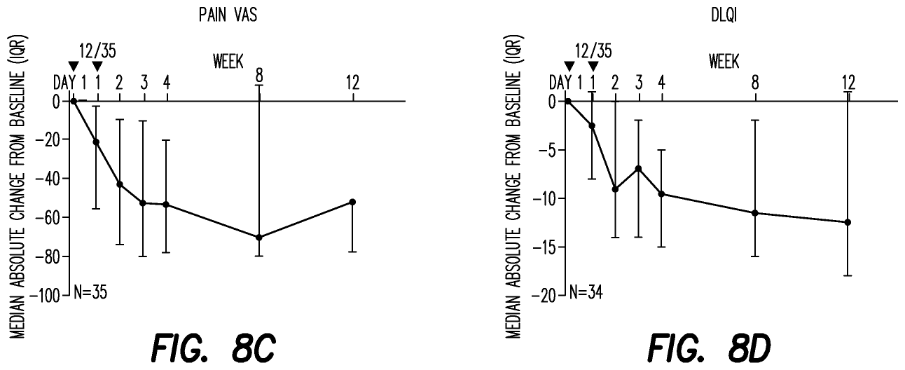

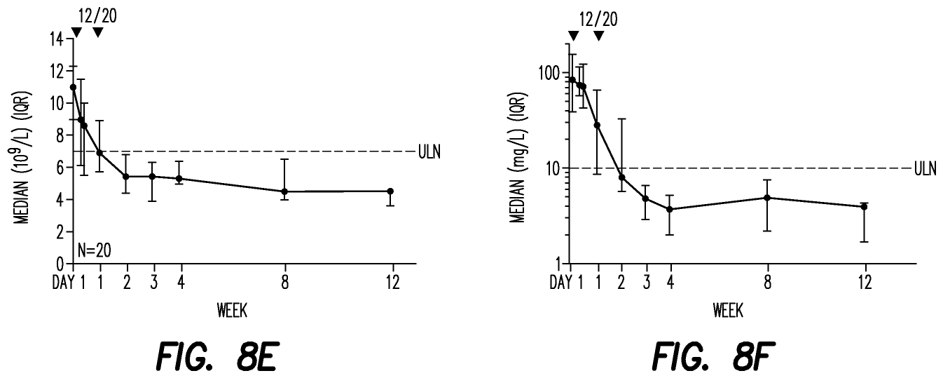

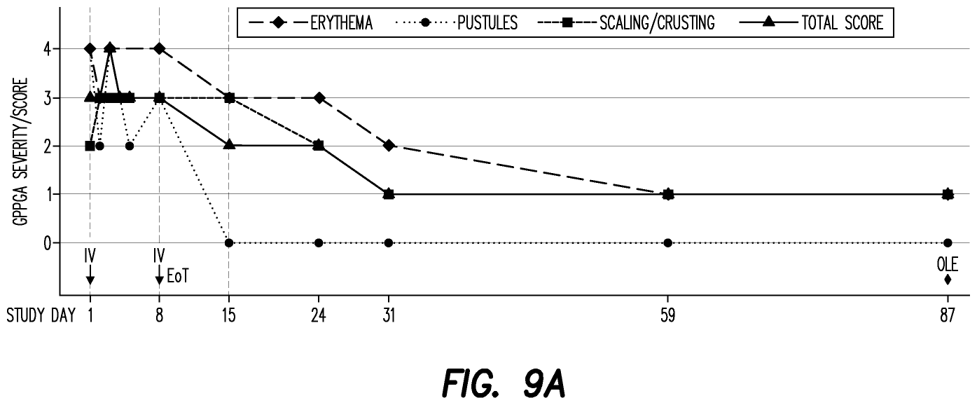

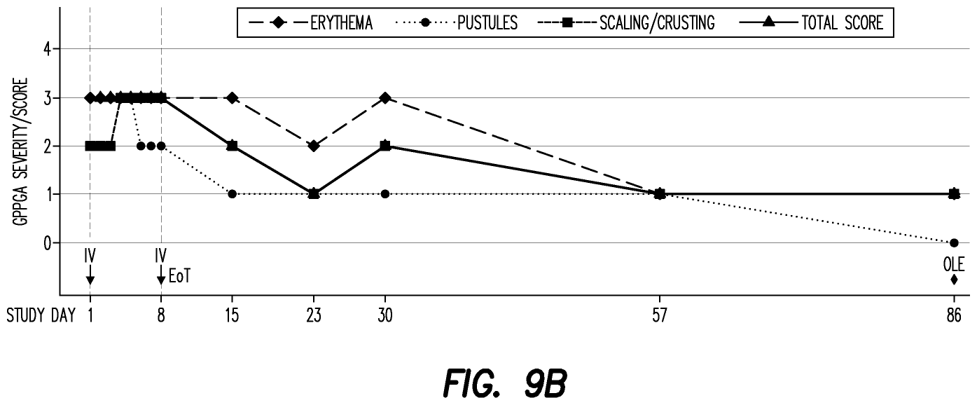

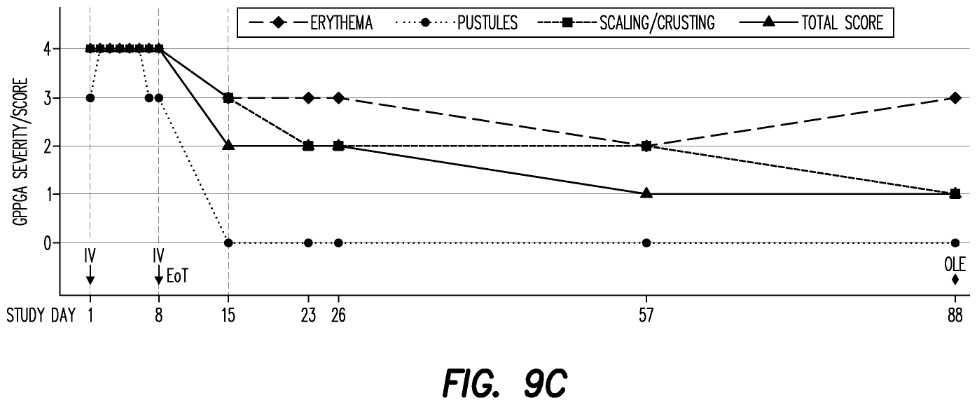

In addition, for the group of patients randomized to spesolimab who received one (day 1) or two doses (day 1 plus day 8; N=35) of spesolimab, descriptive results for GPPASI, GPPASI 75, pain VAS, DLQI, neutrophil counts, and C-reactive protein levels over 12 weeks are reported in FIGS. 8A-8F. Changes from baseline in GPPGA scores (including GPPGA pustulation, erythema and scaling/crusting subscores and GPPGA total score) in three patients after receiving a first and a second dose of spesolimab on days 1 and 8, respectively are shown in FIGS. 9A-9C.

### Safety

Through the first week of randomized treatment, adverse events were reported in 65.7% of patients assigned to spesolimab and 55.6% in the placebo arm. Pyrexia occurred in 5.7% and 22.2% of the patients receiving spesolimab and placebo, respectively; all pyrexia events occurred in the context of the underlying GPP flare but the drug causing pyrexia cannot be excluded (Table 3). Infections were reported in 17.1% and 5.6% of patients in the spesolimab and placebo groups, respectively, through the first week (Supplementary Table S8); At week 1, in the spesolimab group, there were two cases of urinary tract infections, for all other infections, there was one case of each (see footnote to Supplementary Table S8). Serious adverse events were reported in 5.7% of patients with spesolimab and none of the patients receiving placebo.

TABLE 3
Adverse Events Summary

| | Week 1 | | Week 12^{†} |
|---|---|---|---|
| n (%) | Spesolimab | Placebo | Spesolimab |
| [rate/100 patient-years] | (N = 35) | (N = 18) | (N = 51) |
| Any adverse event | 23 (65.7) | 10 (55.6) | 42 (82.4) |
| | [5874.7] | [4623.4] | [981.5] |
| Severe adverse event (RCTC | 2 (5.7) | 1 (5.6) | 5 (9.8) |
| grade 3 or 4) | [309.5] | [304.4] | [40.9] |
| Investigator-defined drug-related | 10 (28.6) | 5 (27.8) | 28 (54.9) |
| adverse event | [1747.6] | [1773.1] | [353.5] |
| Serious adverse event | 2 (5.7) | 0 (0) | 6 (11.8) |
| | [309.5] | [0] | [49.7] |
| Death | 0 | 0 | 0 |
| Adverse event leading to | 0 | 0 | 0 |
| discontinuation of treatment | | | |

Common adverse event^{‡}

| | | | |
|---|---|---|---|
| Pyrexia | 2 (5.7) | 4 (22.2) | 5 (9-8) |
| | [313.5] | [1404.8] | [41.3] |
| Dizziness | 0 | 2 (11.1) | 0 |
| | | [619.1] | |

Serious adverse events

| | | | |
|---|---|---|---|
| Drug reaction with eosinophilia | 1 (2.9) | 0 | 2 (3.9) |
| and systemic symptom | [154.1] | | [15.9] |
| Urinary tract | 1 (2.9) | 0 | 1 (2.0) |
| infection | [154.1] | | [7.8] |
| Drug-induced liver | 1 (2.9) | 0 | 1 (2.0) |
| injury^{§} | [154.1] | | [7.8] |
| Arthritis | 1 (2.9) | 0 | 1 (2.0) |
| | [152.2] | | [7.8] |
| Chronic plaque | 0 | 0 | 1 (2.0) |
| psoriasis worsening^{¶} | | | [7.8] |
| Influenza | 0 | 0 | 1 (2.0) |
| | | | [7.7] |
| Squamous cell carcinoma | 0 | 0 | 1 (2.0) |
| of skin | | | [7.7] |

*All adverse events occurring between start of treatment and end of the residual effect period (16 weeks after the last dose of trial) were considered ‘treatment-emergent’. Adverse events were coded using the Medical Dictionary for Drug Regulatory Activities version 23.1. The adverse event severity was graded according with the Rheumatology Common Toxicity Criteria (RCTC) version 2.0 safety analysis set. Pustular psoriasis was excluded as an adverse event from this safety analysis.
^{†}Dataset at week 12 includes patients randomized to spesolimab who received up to three doses of spesolimab and patients initially randomized to placebo who received open-label spesolimab at or post day 8. All adverse events from the first use of spesolimab to the residual effect period of the last spesolimab dose are included.
^{‡}Common adverse events are reported in ≥ 10% of patients in any treatment group.
^{§}Drug-induced liver injury was reflected by an increase of transaminases and was considered a systemic symptom of drug reaction with eosinophilia and systemic symptom.
^{¶}Chronic plaque psoriasis worsening capture events that were reflective of non-pustular psoriasis; these events were not captured in the efficacy outcomes.

At week 12, 82.4% of patients who received at least one dose of spesolimab (including those initially randomized to placebo who received open-label spesolimab at day 8) had an adverse event, and 11.8% had a serious adverse event; within the spesolimab group, the proportions of patients with adverse events remained unchanged or increased while the time-adjusted incidence rates decreased from week 1 to week 12 (Table 3). Infections were reported in 47.1% of patients; there were three cases each of urinary tract infection and influenza; there were two cases of each: folliculitis, otitis externa, upper respiratory tract infection, pustule; for all other infections, there was one case of each (see footnote to Supplementary Table S8). Symptoms observed in two patients receiving spesolimab were reported as drug reactions with eosinophilia and systemic symptoms (DRESS) with RegiSCAR scores of 1 and 3.33 Further details of these two case reports can be found in the Supplementary information below. Anti-drug antibodies (ADAs) were detected with a median time of 2.3 weeks after spesolimab administration. ADAs were detected in 23/50 (46%) patients who received at least one dose of spesolimab. The majority (87%) of the ADA-positive patients (40% of total treated) were also Nab positive and the NAb status appeared to be associated with the titer value. No dose-dependency observed; however, surprisingly the maximum ADA titers appear lowered in GPP patients (n=4!) who received two doses by Day 8 vs 1 dose only.

### Discussion

This randomized trial of a single intravenous dose of the humanized anti-IL-36 receptor monoclonal antibody, spesolimab in patients with a flare of GPP, showed that at 1 week there was better clearance of lesions compared to placebo. Overall, infections were more frequent with spesolimab, although there was no apparent pattern regarding pathogen and affected organs, and two patients treated with spesolimab had drug reactions with eosinophilia and systemic symptoms with RegiSCAR scores of 1 and 3.

At the end of the randomized 1 week period, a third of patients in the spesolimab group and most patients in the placebo group received open-label spesolimab and were followed for 12 weeks. As 15 of 18 randomized placebo patients received open-label spesolimab, the true effect of spesolimab versus placebo could not be reflected after week 1 in this trial. This highlights one of the inherent challenges of conducting a randomized placebo-controlled trial in patients with this disease; the episodic nature and severity of GPP flares (potentially life threatening) presented several challenges in the design of this study including limiting the duration as to how long patients could ethically be expected to undertake placebo treatment in this trial; further, the clinical course is heterogeneous and can be characterized as a relapsing disease with recurrent flares, or a persistent disease with intermittent flares, making selection of appropriate timings for study endpoints difficult.

As noted above, this trial has limitations, including the small number of enrolled patients (typical for a rare disease); however, the effect size for the primary and key secondary endpoints at week 1 were large and significant. The randomized period was limited to 1 week and the option for patients, at week 1, to receive open-label treatment with spesolimab, if a pre-specified threshold for severity was still met, meant most patients on placebo received spesolimab, and thus comparative analyses after week 1 were non-informative; as such hierarchical tests of secondary endpoints defined at week 4, were not reported here. There were some baseline imbalances between the treatment arms (gender, race, and GPPASI) that occurred at random; however, post hoc sensitivity analyses of the primary and key secondary endpoints adjusted for the imbalances were consistent with the primary analysis.

Despite these challenges, the rapid control of flare episodes in this study with a single infusion of spesolimab, builds on findings from a previous open-label study of spesolimab and supports the hypothesis that IL-36 is a central driver of the pathogenesis of GPP. Improvements over time in measures including CRP, neutrophil counts, pain, PSS, FACIT-Fatigue, DLQI are indicative that spesolimab acts systemically beyond the skin and suggests spesolimab has the potential to improve of the quality of life of patients experiencing a GPP flare. Chronic administration of spesolimab is being evaluated with a subcutaneous formulation in an ongoing 5-year open-label extension (ClinicalTrials.gov identifier: NCT03886246) and the prevention of flares trial Effisayil 2 (ClinicalTrials.gov identifier: NCT04399837).

In conclusion, in this phase 2 trial, GPP flares treated with intravenous spesolimab resulted in higher rates of clearance of pustular lesions compared with placebo but was associated with infections and systemic reactions. Longer and larger trials are required to determine the effect and safety of spesolimab in GPP.

### Supplementary Information

### Generalized Pustular Psoriasis Physician Global Assessment (GPPGA)

GPPGA relies on the clinical assessment of the patient's skin presentation. It is a modified Physician Global Assessment (PGA), a physician's assessment of psoriatic lesions, which has been adapted to the evaluation of patients with generalized pustular psoriasis (GPP). The investigator (or qualified site personnel) scores the erythema, pustules and scaling of all psoriatic lesions from 0 to 4 (see table below). Each component is graded separately, the average is calculated and the final GPPGA is determined from this composite score (composite mean score=(erythema+pustules+scaling)/3; a total GPPGA score of 0 mean=0 for all three components, a score of 1 means 0 to <1.5, score of 2 means 1.5 to <2.5, score of 3 means 2.5 to <3.5, and a score of 4 mean 3.5). A lower score indicates a lesser severity, with 0 relating to ‘clear’ and 1 relating to ‘almost clear’. To receive a score of 0 or 1, the patient should be afebrile in addition to the skin presentation requirements.

### Psoriasis Area and Severity Index for Generalized Pustular Psoriasis (GPPASI)

The GPPASI is an adaptation of the PASI, an established measure of severity and area of psoriatic lesions in patients with psoriasis, for patients with GPP. Similar adaptions have been used for palmoplantar psoriasis. In the GPPASI, the induration component has been substituted by a pustules component. It is a tool that provides a numeric scoring for the patient's overall generalized pustular psoriasis disease state, ranging from 0 to 72. It is a linear combination of the percent of skin surface area (body region area score) that is affected and the severity (scored on a five-point scale, ranging from 0 [least severe] to 4 [most severe]) of erythema, pustules, and scaling (desquamation) over four body regions (head, upper limb, trunk, and lower limb)*.

| Score | Erythema | Pustules | Scaling |
|---|---|---|---|
| 0 | Normal or | No visible | No scaling |
| (clear) | post- | pustules | or crusting |
| | inflammatory | | |
| | hyperpig- | | |
| | mentation | | |
| 1 | Faint, diffuse | Low density | Superficial |
| (almost | pink or | occasional | focal scaling or |
| clear) | slight red | small discrete | crusting restricted |
| | | pustules (non- | to periphery of |
| | | coalescent) | lesions |
| 2 | Light red | Moderate | Predominantly |
| (mild) | | density grouped | fine scaling or |
| | | discrete small | crusting |
| | | pustules (non- | |
| | | coalescent) | |
| 3 | Bright red | High density | Moderate scaling |
| (moderate) | | pustules with | or crusting covering |
| | | some coalescence | most or all lesions |
| 4 | Deep | Very high-density | Severe scaling or |
| (severe) | fiery red | pustules with | crusting covering |
| | | pustular lakes | most or all lesions |

*Individual score per body region = body region factor (head = 0.1, upper limb = 0.2, trunk = 0.3, lower limb = 0.4) × body region area score x sum of component severity scores in body region. Total GPPASI score = sum of individual score from all body regions.

### Inclusion and Exclusion Criteria

### Inclusion Criteria

Patients will be enrolled (screened) into the trial, if they meet the following criteria:

1a. Patients with a GPPGA score of 0 or 1 and a known and documented history of GPP (per ERASPEN criteria) regardless of IL36RN mutation status, and in addition with previous evidence of fever, and/or asthenia, and/or myalgia, and/or elevated C-reactive protein, and/or leukocytosis with peripheral blood neutrophilia (above the upper limit of normal [ULN]), OR
1b. Patients with a flare of moderate-to-severe intensity meeting the ERASPEN criteria of GPP, with a known and documented history of GPP (per ERASPEN criteria) regardless of IL36RN mutation status, and in addition with previous evidence of fever, and/or asthenia, and/or myalgia, and/or elevated C-reactive protein, and/or leukocytosis with peripheral blood neutrophilia (above ULN), OR
1c. Patients experiencing their first episode of a GPP flare of moderate-to-severe intensity with evidence of fever, and/or asthenia, and/or myalgia, and/or elevated C-reactive protein, and/or leukocytosis with peripheral blood neutrophilia (above ULN). For these patients, the diagnosis will be confirmed retrospectively by a central external expert/committee.
2. Patients may or may not be receiving background treatment with retinoids and/or methotrexate and/or cyclosporine. Patients must discontinue retinoids/methotrexate/cyclosporine prior to receiving the first dose of spesolimab or placebo.
3. Male or female patients, aged 18-75 years at screening.
4. Signed and dated written informed consent prior to admission to the trial in accordance with ICH-GCP and local legislation prior to start of any screening procedures.
5. Women of childbearing potential must be ready and able to use highly effective methods of birth control per ICH M3 (R2) that result in a low failure rate of less than 1% per year when used consistently and correctly. A woman is considered of childbearing potential (WOCBP), i.e. fertile, following menarche and until becoming postmenopausal unless permanently sterile. Permanent sterilization methods include hysterectomy, bilateral salpingectomy and bilateral oophorectomy. Tubal ligation is not a method of permanent sterilization. A postmenopausal state is defined as no menses for 12 months without an alternative medical cause.

### Exclusion Criteria

Patients will not be screened or treated if any of the following criteria apply:

1. Patients with synovitis-acne-pustulosis-hyperostosis-osteitis syndrome.
2. Patients with primary erythrodermic psoriasis vulgaris.
3. Patients with primary plaque psoriasis vulgaris without presence of pustules or with pustules that are restricted to psoriatic plaques.
4. Drug-triggered acute generalized exanthematous pustulosis.
5. Immediate life-threatening flare of GPP or requiring intensive care treatment, according to the investigator's judgement. Life-threatening complications mainly include, but are not limited to, cardiovascular/cytokine-driven shock, pulmonary distress syndrome or renal failure.
6. Severe, progressive or uncontrolled hepatic disease, defined as >3-fold ULN elevation in aspartate transaminase or alanine transaminase or alkaline phosphatase, or >2-fold ULN elevation in total bilirubin.
7. Treatment with:
a. Any restricted medication, or any drug considered likely to interfere with the safe conduct of the trial, as assessed by the investigator.
b. Any prior exposure to spesolimab or another IL36R inhibitor.
8. Patients with dose escalation of their maintenance therapy with cyclosporine and/or methotrexate and/or retinoids within the 2 weeks prior to receiving the first dose of spesolimab/placebo.
9. The initiation of systemic agents such as cyclosporine and/or retinoids and/or methotrexate 2 weeks prior to receiving the first dose of spesolimab/placebo.
10. Patients with congestive heart disease, as assessed by the investigator.
11. Active systemic infections (fungal and bacterial disease) during the last 2 weeks prior to receiving first drug administration, as assessed by the investigator.
12. Increased risk of infectious complications (e.g. recent pyogenic infection, any congenital or acquired immunodeficiency [e.g. human immunodeficiency virus (HIV)], past organ or stem cell transplantation), as assessed by the investigator.
13. Relevant chronic or acute infections including HIV or viral hepatitis. For patients screened while having a flare (inclusion criteria 1b or 1c), if Visit 1 HIV or viral hepatitis results are not available in time for randomization, these patients may receive randomized treatment as long as the investigator has ruled out active disease based on available documented history (i.e. negative HIV and viral hepatitis test results) within 3 months prior to Visit 2. A patient can be re-screened if the patient was treated and is cured from acute infection.
14. Active or latent tuberculosis (TB):
QuantiFERON® (or if applicable, T-Spot®) TB test will be performed at screening. If the result is positive, the patient may participate in the trial if further work up (according to local practice/guidelines) establishes conclusively that the patient has no evidence of active tuberculosis. Patients with active TB must be excluded. If presence of latent tuberculosis is established, then treatment should have been initiated and maintained according to local country guidelines. For patients screened while having a flare (inclusion criteria 1 b or 1c), if the TB test results are not available in time for randomization, these patients may receive randomized treatment (provided they meet all other inclusion/exclusion criteria) as long as the investigator has ruled out active disease based on available documented history (i.e. negative for active TB) within 3 months prior to Visit 2.
15. History of allergy/hypersensitivity to a systemically administered trial medication agent or its excipients.
16. Any documented active or suspected malignancy or history of malignancy within 5 years prior to screening, except appropriately treated basal or squamous cell carcinoma of the skin or in situ carcinoma of uterine cervix.
17. Currently enrolled in another investigational device or drug trial, or less than 30 days since ending another investigational device or drug trial(s) or receiving other investigational treatment(s).
18. Women who are pregnant, nursing or who plan to become pregnant while in the trial. Women who stop nursing before the trial drug administration do not need to be excluded from participating; they should refrain from breastfeeding up to 16 weeks after the trial drug administration.
19. Major surgery (major according to the investigator's assessment) performed within 12 weeks prior to receiving the first dose of trial drug or planned during the trial, e.g. hip replacement, aneurysm removal, stomach ligation, as assessed by the investigator.
20. Evidence of a current or previous disease, medical condition (including chronic alcohol or drug abuse or any condition) other than GPP, surgical procedure, psychiatric or social problems, medical examination finding (including vital signs and electrocardiogram) or laboratory value at the screening outside the reference range that in the opinion of the investigator is clinically significant and would make the trial participant unreliable to adhere to the protocol, comply with all trial visits/procedures or to complete the trial, compromise the safety of the patient or compromise the quality of the data.

SUPPLEMENTARY TABLE S3
Efficacy Outcomes and Description

| | | |
|---|---|---|
| Outcome | | |
| measure | Timepoint(s) | Description |

Primary outcome

| | | |
|---|---|---|
| GPPGA | Week 1 | No visible pustules |
| pustulation | | |
| subscore of 0 | | |

Key secondary outcome

| | | |
|---|---|---|
| GPPGA score | Week 1 | Composite mean score = (erythema + pustules + |
| of 0 or 1 | | scaling)/3; total GPPGA score given is 0 if mean is 0, and |
| | | 1 if mean 0 to < 1.5 for all three components: |
| | | Erythema, 0 (normal or post-inflammatory |
| | | hyperpigmentation) to 4 (deep fiery red) |
| | | Pustules, 0 (no visible pustules) to 4 (very high-density |
| | | pustules with pustular lakes) |
| | | Scaling, 0 (no scaling or crusting) to 4 (severe scaling or |
| | | crusting covering most or all lesions) |

Secondary endpoints

| | | |
|---|---|---|
| GPPASI 75 | Week 4 | 75% improvement in GPPASI total score. |
| | | Composite mean score = sum of individual score (as |
| | | defined per GPPGA) from all body regions. Individual |
| | | score per body region = body region factor (head = 0.1, |
| | | upper limb = 0.2, trunk = 0.3, lower limb = 0.4) × body |
| | | region area score × sum of component severity scores in |
| | | body region. Patients' overall GPPASI ranges from 0 to 72 |
| Change from | Week 4 | PRO providing a range of scores from 0 to 100 in a |
| baseline in pain | | continuous visual scale of 100 mm in length to indicate |
| VAS score | | the severity of GPP pain during the previous week. A |
| | | higher score indicates greater pain intensity |
| Change from | Week 4 | PRO providing a range of 0 (none) to 4 (very severe) to |
| baseline in PSS | | assess severity of pain, redness, itching, and burning |
| score | | symptoms during the past 24 hours. The symptom scores |
| | | are added to an unweighted total score, ranging from 0 |
| | | to 16 |
| Change from | Week 4 | PRO consisting of a 13-item questionnaire that assesses |
| baseline in FACIT- | | self-reported fatigue and its impact upon daily activities |
| Fatigue score | | and function during the previous week (7 days). |
| | | Responses of ″not at all″, ″a little″, ″somewhat″, ″quite a |
| | | bit″, and ″very much″ are available for each question, |
| | | and correspond to scores of 0, 1, 2, 3 and 4, respectively. |
| | | The total score ranges from 0 to 52. Items are reversed |
| | | to provide a score in which lower scores indicate greater |
| | | fatigue. |
| GPPGA score | Week 4 | |
| of 0 or 1 | | |
| GPPGA | Week 4 | |
| pustulation | | |
| subscore of 0 | | |
| GPPASI 50 | Weeks 1 and 4 | 50% improvement in GPPASI total score |

CGI, Clinical Global Impression;
DLQI, Dermatology Life Quality Index;
EQ-5D-5L, 5-level EuroQol-5 dimensions;
FACIT, Functional Assessment of Chronic Illness Therapy;
GPP, generalized pustular psoriasis;
GPPASI, Generalized Pustular Psoriasis Area and Severity Index;
GPPGA, Generalized Pustular Psoriasis Physician Global Assessment;
IV, intravenous;
JDA, Japanese Dermatological Association;
OL, open-label;
PRO, patient-reported outcome;
PSS, Psoriasis Symptom Scale;
VAS, visual analog scale.

SUPPLEMENTARY TABLE S4
Absence or Presence of Potential Pathogenic
Variations of CARD14 and
AP1S3 Mutations Based on Genotyping

| | | |
|---|---|---|
| Spesolimab | Placebo | Tot [symbol] |
| (N = 35) | (N = 18) | (N = 5 |

CARD14 mutation^{†}*

| | | | |
|---|---|---|---|
| Yes - no. (%) | 5 (14.3) | 0 (0.0) | 5 (9. |
| No - no. (%) | 24 (68.6) | 14 (77.8) | 38 (71 |
| DNA sequencing | 6 (17.1) | 4 (22.2) | 10 (18 |
| not available - no. (%) | | | |

AP1S3 mutation^{†}

| | | | |
|---|---|---|---|
| Yes - no. (%) | 1 (2.9) | 0 (0.0) | 1 (1. |
| No - no. (%) | 28 (80.0) | 14 (77.8) | 42 (79 |
| DNA sequencing not | 6 (17.1) | 4 (22.2) | 10 (18 |
| available - no. (%) | | | |

At the date of database lock (18 Jan. 2021), DNA sequencing (targeted re-sequencing Illumina Miseq, Illumina Inc., San Diego, CA) was not yet completed in three patients and there were missing samples from seven patients.
*Positive patients had heterozygous mutations (two p.Ser200Asn; one p.Arg682Trp; one p.Pro506Leu; one p.Ser539Arg). One patient in the spesolimab arm carried double CARD14 and IL36RN mutations (p.Ser200Asn and p.Ser113Leu, respectively).
^{†}The single positive patient had a heterozygous mutation (p.Phe4Cys). One patient in the spesolimab arm carried double AP1S3 and IL36RN mutations (p.Phe4Cys and p.Leu27Pro, respectively). [symbol]
indicates data missing or illegible when filed

SUPPLEMENTARY TABLE S5
Post-hoc Sensitivity Analysis for the Primary Endpoint with
Linear Regression Adjusting for Gender, Race, and Baseline GPPASI

| | Speso- | | Speso- | Pla- |
|---|---|---|---|---|
| | limab | Placebo | limab | cebo [symbol] |
| Gender | Female | | Male | |
| Responders/total patients | 11/21 | 1/15 | 8/14 | 0/3 |
| Adjusted risk | 0.482 (0.269, 0.695) | | | |
| difference (95% CI)* | | | | |
| P-value for treatment^{†} | <0.001 | | | |
| Race | Asian | | White | |
| Responders/total patients | 10/16 | 1/13 | 9/19 | 0/5 |

| | |
|---|---|
| Adjusted risk | 0.522 (0.314, 0.729) |
| difference (95% CI)* | |
| P-value for treatment^{†} | <0.001 |

Baseline GPPASI value

| | |
|---|---|
| Adjusted risk | 0.469 (0.254, 0.683) |
| difference (95% CI)* | |
| P-value for treatment^{†} | <0.001 |

*Based on a linear regression model using the primary endpoint as the dependent variable, and treatment assignment plus the respective covariate as independent variable. 95% CI and P-value based on robust SE.
^{†}P-values for treatment difference are based on a linear regression model.
CI, confidence interval;
GPPSI, Psoriasis Area and Severity Index for Generalized Pustular Psoriasis;
SE, standard error. [symbol]
indicates data missing or illegible when filed

SUPPLEMENTARY TABLE S6
Post-hoc Sensitivity Analysis for the Key Secondary Endpoint with
Linear Regression Adjusting for Gender, Race, and Baseline GPPASI

| | Spesolimab | Placebo | Spesolimab | Placeb [symbol] |
|---|---|---|---|---|
| Gender | Female | | Male | |
| Responders/total patients | 10/21 | 2/15 | 5/14 | 0/3 |
| Adjusted risk difference | 0.346 (0.113, 0.579) | | | |
| (95% CI)* | | | | |
| P-value for treatment^{†} | 0.005 | | | |
| Race | Asian | | White | |
| Responders/total patients | 8/16 | 2/13 | 7/19 | 0/5 |

| | |
|---|---|
| Adjusted risk difference | 0.354 (0.125, 0.583) |
| (95% CI)* | |
| P-value for treatment^{†} | 0.004 |

Baseline GPPASI value

| | |
|---|---|
| Adjusted risk difference | 0.297 (0.058, 0.535) |
| (95% CI)* | |
| P-value for treatment^{†} | 0.018 |

*Based on a linear regression model using the primary endpoint as the dependent variable, and treatment assignment plus the respective covariate as independent variable. 95% CI and P-value based on robust SE.
^{†}P-values for treatment difference are based on a linear regression model.
CI, confidence interval;
GPPSI, Psoriasis Area and Severity Index for Generalized Pustular Psoriasis;
SE, standard error. [symbol]
indicates data missing or illegible when filed

### Exploratory Efficacy Endpoints Post Day 8 by Randomized Treatment at Day 1 and Open Label Spesolimab Treatment at Day 8. Descriptive Analyses for GPPGA Pustulation Subscore of 0 and GPPGA Total Score of 0 or 1 by Four Groups:

In patients, originally randomized to spesolimab, who received spesolimab on day 1 with or without open-label spesolimab on day 8 (N=35), 19 patients (54%) achieved a GPPGA pustulation subscore of 0 and 15 patients (43%) achieved a GPPGA total score of 0 or 1 at the end of week 1; at week 12, 21 patients (60%) had a GPPGA pustulation subscores of 0 and GPPGA total scores of 0 or 1 (FIGS. 6A and 7A). Of the 23 patients randomized to spesolimab who did not receive open-label spesolimab on day 8, 19 (83%) achieved a GPPGA pustulation subscore of 0 and 15 (65%) achieved a GPPGA total score of 0 or 1 at the end of week 1; at week 12, these scores were reported in 15 (65%) and 16 (70%) patients, respectively (FIGS. 6B and 7B). In patients, originally randomized to spesolimab, who received open-label spesolimab on day 8 (N=12), five patients (42%) achieved a GPPGA pustulation subscore of 0 and two patients (17%) achieved a GPPGA total score of 0 or 1 at the end of week 2; at week 12, six patients (50%) and seven patients (58%) had GPPGA pustulation subscores of 0 and GPPGA total scores of 0 or 1 (FIGS. 6C and 7C). Of the 15 patients in the placebo group who received open-label spesolimab on day 8, 11 (73%) achieved a GPPGA pustulation subscore of 0 and eight (53%) achieved a GPPGA total score of 0 or 1 at the end of week 2 (seven days post-open-label spesolimab); at week 12, these scores were reported in six (40%) and eight (53%) patients, respectively (FIGS. 6D and 7C).

Descriptive Analyses for the Planned Hierarchical Secondary Endpoints GPPASI 75 and Change from Baseline in: Pain VAS; PSS; and FACIT-Fatigue at Week 4 by Four Groups

For GPPASI 75 at week 4, in patients, originally randomized to spesolimab, who received spesolimab on day 1 with or without open-label spesolimab on day 8, 18/35 (51%) achieved a GPPASI 75. Of the 23 patients randomized to spesolimab who did not receive open-label spesolimab on day 8, 16 (70%) achieved a GPPASI 75 at week 4 (Supplementary Table S7). Of the 12 patients in the spesolimab group and 15 patients in the placebo group who received an open-label dose of spesolimab at the end of 1 week, two (17%) and six (40%) patients, respectively, achieved a GPPASI 75 at week 4 (Supplementary Table S7).

For pain VAS at week 4, in patients, originally randomized to spesolimab, who received spesolimab on day 1 with or without open-label spesolimab on day 8, there was a median (interquartile range; IQR) change from baseline of −53.4 (−77.8, −20.2) (Supplementary Table S7). Of the 23 patients randomized to spesolimab who did not receive open-label spesolimab on day 8, there was a median (IQR) change from baseline of −63.1 (−79.8, −22.5). Of the 12 patients in the spesolimab group and 15 patients in the placebo group who received an open-label dose of spesolimab there were median (IQR) changes from baseline of −44.6 (−71.2, −17.3) and −54.3 (−79.0, 33.3), respectively (Supplementary Table S7).

For PSS at week 4, in patients, originally randomized to spesolimab, who received spesolimab on day 1 with or without open-label spesolimab on day 8, there was a median (IQR) change from baseline of −7.0 (−10.0, −3.0) (Supplementary Table S7). Of the 23 patients randomized to spesolimab who did not receive open-label spesolimab on day 8, there was a median (IQR) change from baseline of −7.0 (−11.0, −2.0). Of the 12 patients in the spesolimab group and 15 patients in the placebo group who received an open-label dose of spesolimab there were median (IQR) changes from baseline of −6.0 (−7.0, −3.5) and −5.0 (−9.0, −2.0), respectively (Supplementary Table S7).

For FACIT-Fatigue at week 4, in patients originally randomized to spesolimab, who received spesolimab on day 1 with or without open-label spesolimab on day 8, there was a median (IQR) change from baseline of 22.0 (1.0, 31.0) (Supplementary Table S7). Of the 23 patients randomized to spesolimab who did not receive open-label spesolimab on day 8, there was a median (IQR) change from baseline of 22.0 (3.0, 35.0) (Supplementary Table S7). Of the 12 patients in the spesolimab group and 15 patients in the placebo group who received an open-label dose of spesolimab there were median (IQR) changes from baseline of 15.0 (5.5, 28.5) and 16.0 (−19.0, 26.0), respectively (Supplementary Table S7).

SUPPLEMENTARY TABLE 7
GPPASI 75, Pain VAS, PSS, and FACIT-Fatigue at
Week 1 and 4 by Randomized Treatment at Day 1 and
Open-Label Spesolimab Treatment at Day 8.

| GPPASI 75 | | | |
|---|---|---|---|
| Proportion of patients - no. (%) | Baseline | Week 1 | |
| (95% CI) | (day 1) | (day 8) | Week 4 |
| Spesolimab randomized all (N = 35) | — | 4 (11.4) | 18 (51.4) |
| | | (4.5, 26.0) | (35.6, 67.0) |
| Spesolimab randomized 900 mg single | — | 4 (17.4) | 16 (69.6) |
| dose only (N = 23) | | (7.0, 37.1) | (49.1, 84.4) |
| Spesolimab randomized 900 mg single | — | 0 (0.0) | 2 (16.7) |
| dose + open label day 8 spesolimab | | (0.0, 24.2) | (4.7, 44.8) |
| (N = 12) | | | |
| Placebo randomized + open label | — | 0 (0.0) | 6 (40.0) |
| day 8 spesolimab (N = 15) | | (0.0, 20.4) | (19.8, 64.3) |
| | | Week 1 (day 8) | Week 4 |
| | Baseline | Median absolute | Median absolute |
| | (day 1) | change from | change from |
| Pain VAS | Median (IQR) | baseline (IQR) | baseline (IQR) |
| Spesolimab randomized all (N = 35) | 79.8 | −21.3 | −53.4 |
| | (20, 100) | (−55.3, −3.1) | (−77.9, −20.2) |
| Spesolimab randomized 900 mg single | 79.8 | −27.6 | −63.1 |
| dose only (N = 23) | (62.8, 87.8) | (−56.3, −9.6) | (−79.8, −22.5) |
| Spesolimab randomized 900 mg single | 78.7 | −9.2 | −44.6 |
| dose + open label day 8 spesolimab | (75.3, 86.0) | (−36.5, −1.0) | (−71.2, −17.3) |
| (N = 12) | | | |
| Placebo randomized + open label | 71.9 | −18.8 | −54.3 |
| day 8 spesolimab (N = 15) | \|(50.0, 89.4) | (−38.8, 18.0) | (−79.0, 33.3) |
| | | Week 1 (day 8) | Week 4 |
| | Baseline | Median absolute | Median absolute |
| | (day 1) | change from | change from |
| PSS | Median (IQR) | baseline (IQR) | baseline (IQR) |
| Spesolimab randomized all (N = 35) | 11.0 | −4.0 | −7.0 |
| | (3.0, 16.0) | (−7.0, 0.0) | (−10.0, −3.0) |
| Spesolimab randomized 900 mg single | 11.0 | −5.0 | −7.0 |
| dose only (N = 23) | (9.0, 12.0) | (−8.0, −1.0) | (−11.0, −2.0) |
| Spesolimab randomized 900 mg single | 10.5 | 0.0 | −6.0 |
| dose + open label day 8 spesolimab | (9.0, 13.5) | (−3.5, 2.5) | (−7.0, −3.5) |
| (N = 12) | | | |
| Placebo randomized + open label | 10.0 | −1.0 | −5.0 |
| day 8 spesolimab (N = 15) | (9.0, 11.0) | (−5.0, 2.0) | (−9.0, −2.0) |
| | | Week 1 (day 8) | Week 4 |
| | Baseline | Median absolute | Median absolute |
| | (day 1) | changefrom | change from |
| FACIT-Fatigue | Median (IQR) | baseline (IQR) | baseline (IQR) |
| Spesolimab randomized all (N = 35) | 14.0 | 7.0 | 22.0 |
| | (1.0, 49.0) | (1.0, 20.0) | (1.0, 31.0) |
| Spesolimab randomized 900 mg single | 13.0 | 12.0 | 22.0 |
| dose only (N = 23) | (6.0, 31.0) | (2.0, 20.0) | (3.0, 35.0) |
| Spesolimab randomized 900 mg single | 17.0 | 4.5 | 15.0 |
| dose + open label day 8 spesolimab | (10.0, 25.5) | (0.5, 16.5) | (5.5, 28.5) |
| (N = 12) | | | |
| Placebo randomized + open label | 17.0 | 3.0 | 16.0 |
| day 8 spesolimab (N = 15) | (6.0, 33.0) | (−3.0,11.0) | (−19.0, 26.0) |

This exploratory dataset includes the analyses originally planned for hierarchical testing of secondary endpoints (GPPASI 75 and change from baseline in: pain VAS; PSS; and FACIT-Fatigue) all at week 4, in the statistical analysis plan; however, randomization to trial groups no longer pertained after week 1 as 15 of 18 patients assigned to placebo received open-label spesolimab on day 8. For this analysis, any values post open-label spesolimab at day 8 are used, but any values post use of escape medication, or rescue medication with spesolimab are not used and imputed as the worst outcome in the calculation of median and quartiles.
CI, confidence interval;
GPPASI 75, 75% or greater improvement in the Psoriasis Area and Severity Index for Generalized Pustular Psoriasis;
FACIT-Fatigue, Functional Assessment of Chronic Illness Therapy-Fatigue;
IQR, interquartile range;
Pain VAS, pain visual analog scale;
PSS, Psoriasis Symptom Scale.

### Descriptive Analyses for GPPASI, GPPASI 75, Pain VAS, DLQI, Neutrophil Counts and C-Reactive Protein Levels by the Group of Patients Randomized to Spesolimab Who Received One (Day 1) or Two Doses (Day 1 Plus Day 8); N=35) of Spesolimab:

The median percent improvement in the GPPASI score from baseline was 43% at week 1 and progressively increased up to 82% at week 12 (FIG. 8A). A GPPASI 75 was achieved in 13 patients (37%) at week 2, in 18 patients (51%) at week 4, 21 patients (60%) at week 8 and 20 patients (57%) at week 12 (FIG. 8B). Rapid reductions in pain VAS score and improvements in DLQI scores were achieved through week 4 and through week 12 (FIGS. 8C and 8D). In patients, with elevated baseline neutrophils, counts were normalized within 1 week of receiving spesolimab (FIG. 8E). Median C-reactive protein (CRP) normalized within 2 weeks in patients with elevated baseline CRP 10 mg/L), who received one or two doses of spesolimab (FIG. 8F).

SUPPLEMENTARY TABLE S8
Summary of Adverse Events by System Organ Class

| | Week 1 | | Week 12* |
|---|---|---|---|
| n (%) | Spesolimab | Placebo | Spesolimab |
| [rate/100 patient-years] | (N = 35) | (N = 18) | (N = 51) |
| Adverse events by | | | |
| System Organ Class¹ | | | |
| Skin and subcutaneous tissue | 5 (14.3) | 2 (11.1) | 14 (27.5) |
| disorders | [822.6] | [593.9] | [137.9] |
| Infections and infestations^{‡} | 6 (17.1) | 1 (5.6) | 24 (47.1) |
| | [987.2] | [292.2] | [262.8] |
| General disorders and | 9 (25.7) | 5 (27.8) | 13 (25.5) |
| administration site conditions | [1543.3] | [1756.0] | [127.5] |
| Investigations | 4 (11.4) | 2 (11.1) | 7 (13.7) |
| | [664.1] | [619.1] | [61.5] |
| Blood and lymphatic system | 1 (2.9) | 2 (11.1) | 4 (7.8) |
| disorders | [154.1] | [640.8] | [32.9] |
| Metabolism and nutrition | 3 (8.6) | 2 (11.1) | 4 (7.8) |
| disorder | [484.8] | [624.4] | [32.4] |
| Nervous system disorders | 4 (11.4) | 3 (16.7) | 7 (13.7) |
| | [655.2] | [961.2] | [60.5] |
| Musculoskeletal and | 4 (11.4) | 2 (11.1) | 11 (21.6) |
| connective tissue | [627.0] | [624.4] | [101.6] |
| Gastrointestinal disorder | 2 (5.7) | 1 (5.6) | 13 (25.5) |
| | [309.5] | [292.2] | [118.5] |

All AEs occurring between start of treatment and end of the residual effect period (16 weeks after the last dose of trial) were considered ‘treatment-emergent’. AEs were coded using the Medical Dictionary for Drug Regulatory Activities version 23.1. The AE severity was graded according with the Rheumatology Common Toxicity Criteria version 2.0 safety analysis set. Pustular psoriasis was excluded as an adverse event from this safety analysis.
*Dataset at week 12 includes patients randomized to spesolimab who received up to three doses of spesolimab, and patients initially randomized to placebo who received open-label spesolimab at day 8. All adverse events from the first use of spesolimab to the residual effect period of the last use of spesolimab are included.
†Adverse AEs by System Organ Class are reported in ≥ 10% of patients in any treatment group.

### Example 2

Spesolimab Treatment Improves Pain, Symptoms of Psoriasis, Fatigue and Quality of Life in Patients with Generalized Pustular Psoriasis: Patient-Reported Outcomes Results from the Effisayil 1 Study

Introduction & Objectives: Generalized Pustular Psoriasis (GPP) is a rare and potentially life-threatening skin disease, characterised by widespread sterile pustules and recurrent flares. Signs and symptoms associated with GPP include pain, fever and fatigue, which can impact activities of daily living and overall quality of life (QoL). Spesolimab has been reported to rapidly improve pustular and skin clearance in patients presenting with a GPP flare. The objective of this analysis was to evaluate patient reported outcomes (PROs) on measures of pain, symptoms of psoriasis, fatigue and impact on overall QoL in patients treated with spesolimab from the Effisayil 1 study.

Materials & Methods: Effisayil 1 (NCT03782792) was a multicentre, randomised, double-blind, placebo-controlled study in patients with a GPP flare. Eligible patients (n=53) were randomly assigned (2:1) to receive a single intravenous (IV) dose of open-label 900 mg spesolimab or placebo on Day 1, followed for 12 weeks. At Week 1, patients were eligible to receive an open-label, single IV dose of 900 mg spesolimab if they had a GPP Physician Global Assessment (GPPGA) score ≥2 and did not receive escape medication before day 8. The secondary and further endpoints were assessed by the Psoriasis Symptom Scale (PSS; 0-16, with higher scores indicating more severe symptoms), pain Visual Analogue Scale (pain VAS; 0 [no pain] to 100 [severe pain]), Functional Assessment of Chronic Illness Therapy-Fatigue (FACIT-Fatigue; 0-52, with lower scores indicating greater impact) and the Dermatology Life Quality Index (DLQI; 0 [no effect] to 30 [extremely large effect]) over time through to the end of the study. Patients who received escape treatment (an optional standard of care treatment administered to patients within 1 week of their first dose) or rescue medication (an optional 900 mg IV dose of spesolimab administered anytime from Day 8 onwards) are considered as non-responders for later timepoints in analysis.

Results: Patients were randomised to receive 900 mg spesolimab (n=35) or placebo (n=18). At baseline, patients had a high clinical burden and impaired QoL as indicated by median PSS (11.0), pain VAS (79.8), FACIT-Fatigue (14.0) and DLQI (19.5) scores (Table 9). Improvements in PSS, Pain VAS, FACIT-Fatigue and DLQI scores were observed from baseline to Week 12 (Table). Pain improved as early as Week 1, with further improvements at Week 4 that were maintained to Week 12. For the other PROs (PSS, FACTI-Fatigue and DLQI), improvements were also seen as early as Week 1 but continued to improve through to Week 12.

Conclusions: Patients receiving 900 mg spesolimab IV and an optional second dose at day 8 showed marked improvements in PROs of pain, symptoms of psoriasis, fatigue and overall QoL. These improvements were seen early and were maintained for up to 12 weeks. These findings suggest that up to 2 doses of spesolimab can result in rapid pustular clearance of GPP flares which were associated with improvements in patient-reported outcomes.

TABLE 9
Baseline and absolute change from baseline
in PROs of the spesolimab arm (n = 35)

| Median | Patient-Reported Outcomes | | | | | | | |
|---|---|---|---|---|---|---|---|---|
| (IQR) | PSS | | Pain-VAS | | FACIT-Fatigue | | DLQI | |
| Baseline | 11.0 | (9.0-12.0) | 79.8 | (70.5-87.8) | 14.0 | (7.0-28.0) | 19.5 | (16.0-25.0) |
| Week 1 | −4.0 | (−7.0, 0.0) | −21.3 | (−55.3, −3.1) | 7.0 | (1.0, 20.0) | −2.5 | (−8.0, 1.0) |
| Week 4 | −7.0 | (−10.0, −3.0) | −53.4 | (−77.9, −20.2) | 20.0 | (4.0, 34.0) | −9.5 | (−15.0, −5.0) |
| Week 12 | −5 | (−10.0, NR) | −52.6 | (−77.9, NR) | 24.0 | (NR, 32.0) | −12.5 | (−18.0, 1.0) |

All datasets include patients [symbol] to spesolimab who received up to two doses of spesolimab, including 12 patients who received an open-label, single intravenous does of 900 mg spesolimab (second dose) at week 1.
Any values [symbol] open label spesolimab at week 1 are used, but any use of [symbol] medication, or rescue medication with spesolimab represents non-response.
IQR, interquartile range [symbol]
indicates data missing or illegible when filed

### Example 3

Spesolimab Treatment Improves Pain, Symptoms of Psoriasis, Fatigue and Quality of Life in Patients with Generalized Pustular Psoriasis: Patient-Reported Outcomes from the Effisayil 1 Study

PURPOSE: To evaluate PROs on measures of pain, symptoms of psoriasis, fatigue and impact on overall quality of life (QoL) in patients treated with spesolimab in the Effisayil™ 1 study.

INTRODUCTION: GPP is a rare, potentially life-threatening, neutrophilic skin disease characterised by widespread eruption of sterile, visible pustules, and can occur with or without systemic inflammation. In the multicentre, randomised, double-blind, placebo-controlled Effisayil™ 1 study (NCT03782792) in patients presenting with a GPP flare, spesolimab treatment led to rapid pustular and skin clearance within 1 week. GPP flares are associated with a high clinical burden in PROs including symptoms such as pain, itching and fatigue, which all impact the patient's overall QoL. Here we explore PROs in patients with a GPP flare receiving spesolimab treatment.

METHODS: Patients (N=53) were randomised (2:1) to receive placebo or 900 mg of spesolimab on Day 1 and were followed for 12 weeks. Patients were able to receive standard of care (SoC) treatment any time after receiving their first dose of spesolimab or placebo on Day 1 and before Day 8. Patients who did not receive SoC treatment during Week 1 were eligible to receive open-label (OL) spesolimab on Day 8 and one rescue dose of OL spesolimab between Day 8 and Week 12.

Spesolimab arm (n=35): One dose of spesolimab at Day 1 (n=23); optional second dose of OL spesolimab at Day 8 (n=12); optional third dose of OL spesolimab between Weeks 1 and 12 (n=2).

Placebo arm (n=18): optional first dose of OL spesolimab at Day 8 (n=15); optional second dose of OL spesolimab between Weeks 1 and 12 (n=1).

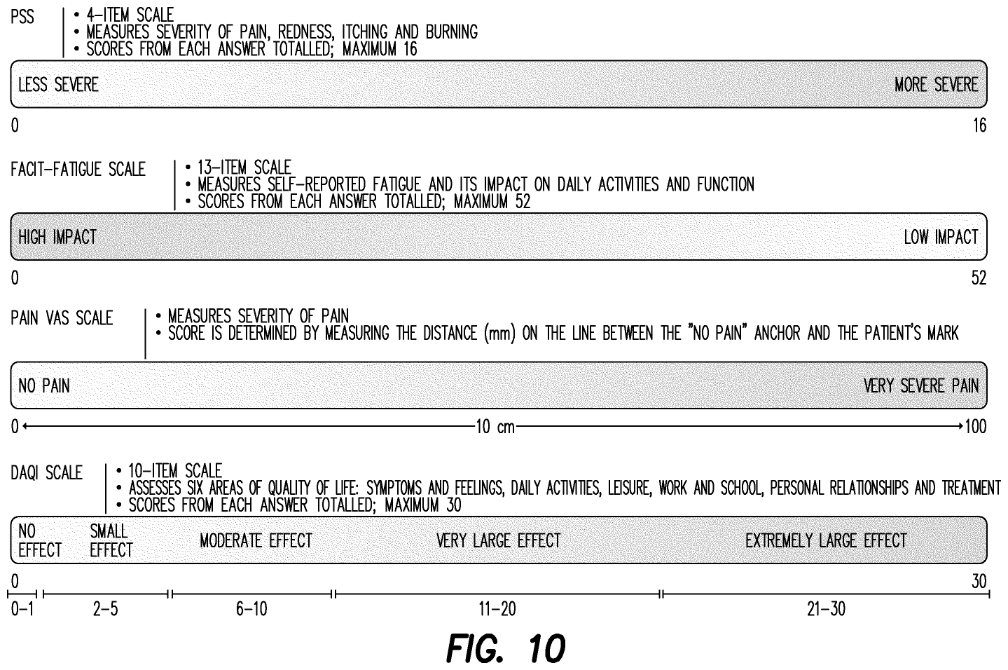

Patients who already received their one rescue dose of spesolimab were treated with escape treatment (SoC) for any subsequent flares. All randomised patients were included in this analysis. The observed cases irrespective of any use of escape treatment, OL spesolimab on Day 8 or rescue spesolimab treatment after Day 8 (representing the intention-to-treat principle) are summarised descriptively. To monitor any changes in outcomes, patients completed the following PRO questionnaires throughout the study: Psoriasis Symptom Scale [PSS], pain visual analogue scale (pain VAS), Functional Assessment of Chronic Illness Therapy— Fatigue (FACIT-Fatigue) and the Dermatology Life Quality Index (DLQI; FIG. 10). All four PROs were measured on Day 1, Day 8 and Weeks 2-4, 8 and 12. PSS scores were also measured every day during Week 1 (data for days 4-7 not shown).

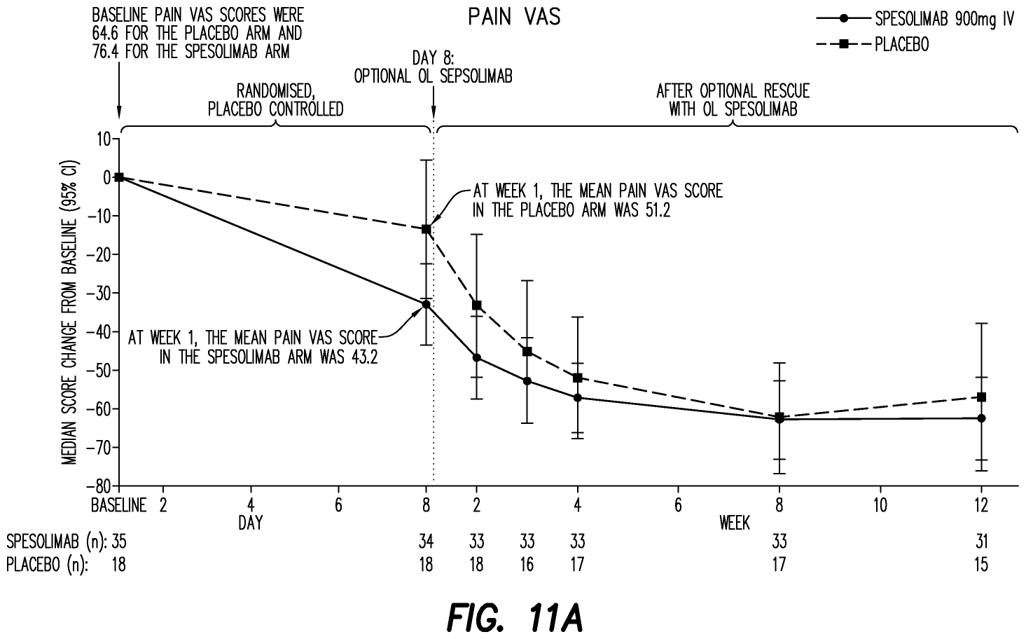

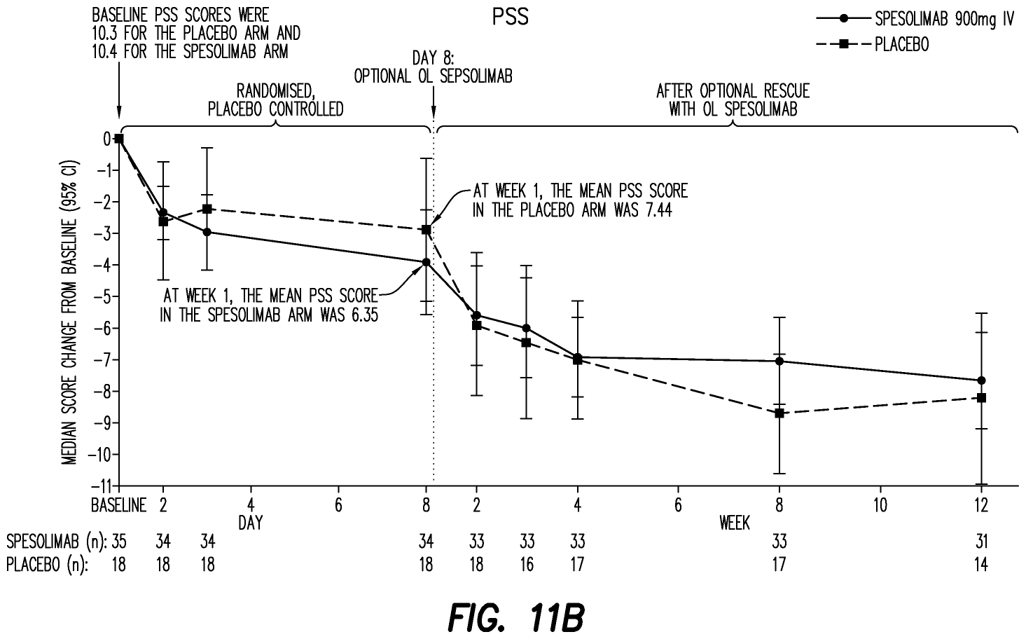

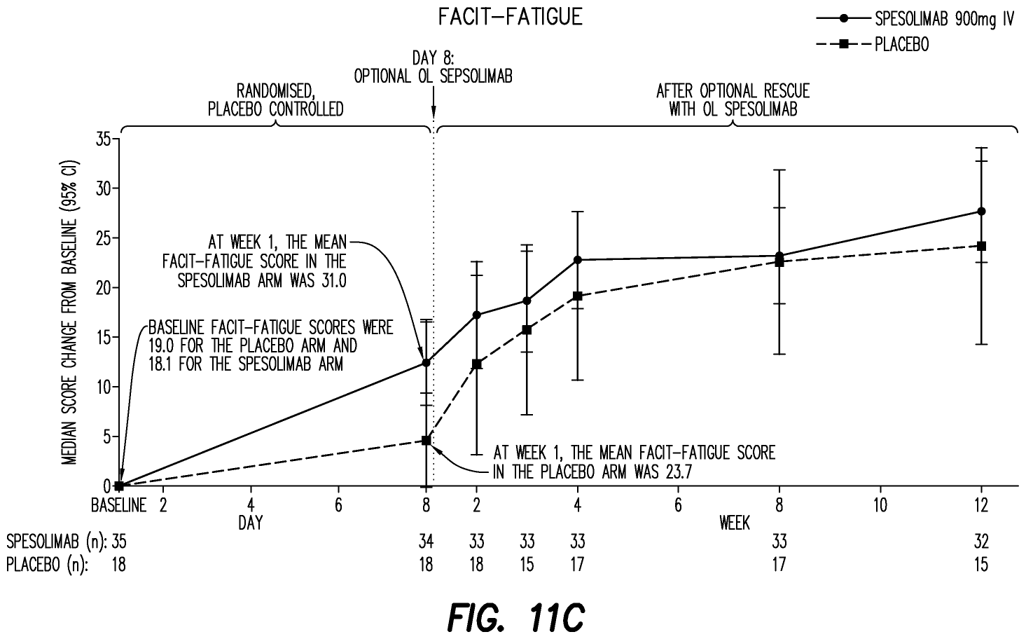

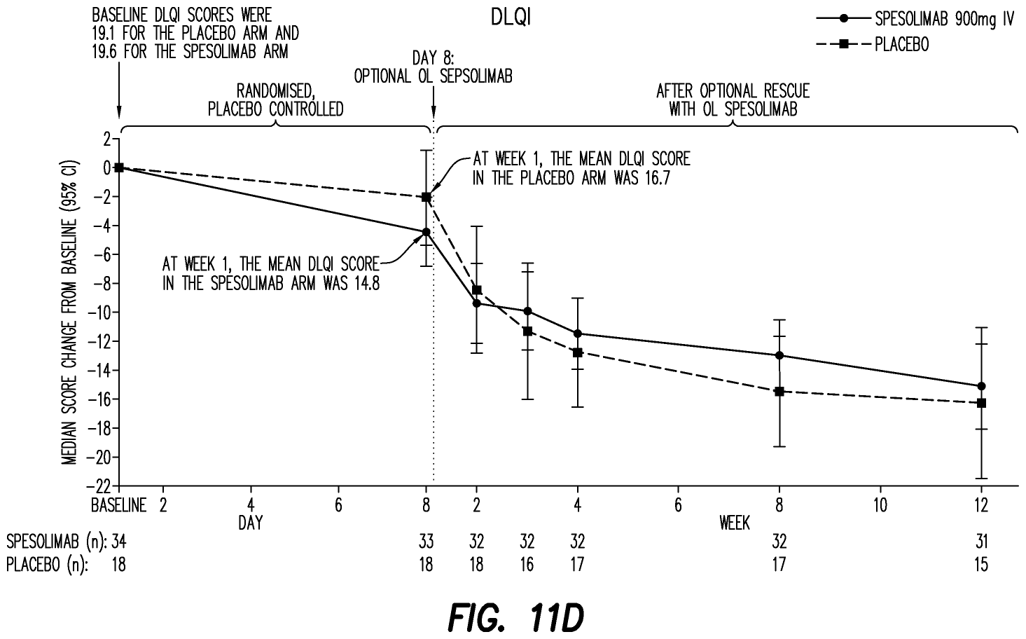

CONCLUSION: In this study, patients who received 900 mg of intravenous spesolimab showed clinically significant improvements from baseline in the PROs of pain, symptoms of psoriasis, fatigue and overall quality of life. The clear separation of the spesolimab and placebo curve occurred early during the placebo-controlled period (Week 1) (FIGS. 11A-11D). This suggests that spesolimab results in the rapid improvement of PROs with considerable improvement in fatigue and pain. PRO scores continued to improve up to Week 4 and were sustained through to Week 12 (FIGS. 11A-11D).

### Example 4

Sustained Treatment Effect of Spesolimab Over 12 Weeks for Generalized Pustular Psoriasis Flares; Results from the Effisayil-1 Study

The objective of this study was to analyze the effects of spesolimab over 12 weeks for treatment of patients presenting with a flare of generalized pustular psoriasis.

In Effisayil™ 1, a double-blind, randomized, placebo-controlled study in patients presenting with a flare of generalized pustular psoriasis (GPP), a rare, life-threatening autoinflammatory disease, spesolimab, an anti-IL-36 receptor antibody, led to rapid clearance (within 1-week) of pustular and skin lesions. Here, we explore the effects of spesolimab over the 12-week duration of the study (intention-to-treat analysis).

Patients (n=53) were randomized to receive a single intravenous dose of 900 mg spesolimab (n=35) or placebo (n=18) on Day 1. At Day 8, patients were eligible to receive an open-label, dose of spesolimab if they presented persistent symptoms; comprising 12 in the spesolimab group (34.3%) and 15 (83.3%) placebo. Of patients initially randomized to spesolimab who received up to two doses (n=35), 61.8% and 84.4% achieved a GPPGA pustulation subscore of 0, and 50.0% and 81.3% a GPPGA total score of 0/1 by Weeks 1 and 12, respectively. Of patients initially randomized to placebo that received open-label spesolimab at Day 8, 83.3% and 80.0% had a GPPGA pustulation subscore of 0 and 72.2% and 93.3% had a GPPGA total score of 0/1 by Weeks 2 (1-week post-spesolimab) and 12, respectively. After Day 8, 32 and 17 patients randomized to spesolimab and placebo respectively, completed the 12-week follow-up period, during which four and two patients, required rescue treatment with spesolimab for a new flare episode.

Spesolimab demonstrated rapid and sustained clinical improvements over 12 weeks. These data further support spesolimab as a potential therapeutic option for patients with a GPP flare.

### Example 5

Results of Clinical Immunogenicity Evaluation—Trial 1368-0013: Testing Spesolimab (BI 655130) in Patients with a Flare-Up of a Skin Disease Called Generalized Pustular Psoriasis

Analysis methods: Blood samples for assessment of ADA/NAb were collected from subjects (patients) before the initiation and during the course of spesolimab treatment, and at end of study/discontinuation. All samples were first analyzed in the ADA screening assay and only those found to be putative positive were subsequently assessed in the ADA confirmatory assay. Only samples that confirmed positive were titrated to obtain a titer value. In addition, development of NAbs was evaluated only in subjects with ADA-positive samples.

Evaluation of spesolimab immunogenicity was performed using data from all evaluable subjects, defined as subjects who had a baseline immunogenicity assessment and at least 1 post-baseline value. For spesolimab treated subjects, baseline samples were considered to be the last sample obtained before initiation of active treatment. For patients who were not treated with spesolimab, baseline samples were the last sample before receiving placebo treatment.

Trial background: This was a randomized, placebo-controlled, double-blind, parallel-group, single-dose trial with 2 treatment groups (900 mg i.v. spesolimab and placebo on Day 1). Patients could qualify to receive an open-label (OL) treatment with 900 mg i.v. spesolimab on Day 8 and rescue treatment with spesolimab after Day 8, depending on their GPPGA total score and the GPPGA pustulation subscore. Patients were offered to roll over into the open-label extension (OLE) trial 1368-0025 if they met the inclusion criteria of this OLE trial. The follow-up period of trial 1368-0013 was 12 to 28 weeks, depending on the timing of the last spesolimab dose in trial 1368-0013 and on whether patients continued in the OLE trial.

A total of 53 male and female patients with GPP were randomized in a 2:1 ratio to receive a single dose of spesolimab 900 mg i.v. (35 patients) or placebo (18 patients) on Day 1. Spesolimab as open-label dose on Day 8 was administered to 27 patients overall and as rescue medication after Day 8 to a total of 6 patients.

More female (N=36) than male (N=17) patients were randomized. All patients were Asian (N=29) or White (N=24). The mean (StD) age was 43.0 (10.9) years and the mean (StD) weight was 72.0 (24.7) kg.

PK and ADA/NAb samples were collected predose, Day 4, Week 1, 2, 3, 4, 8, and end of study. For patients who did not enter the OLE 1368-0025 trial, the End-of-study Visit is 16 weeks after the last spesolimab dose. For patients who entered the OLE trial, the End-of-study Visit is Week 12, or 6 weeks after the last dose if patient received a rescue dose during Week 7-12.

Anti-drug antibody/Neutralizing antibody (ADA/Nab) Responses: In trial 1368-0013, out of 53 randomized patients, 2 patients who were randomized to placebo on Day 1 did not receive any spesolimab treatment during the trial. All ADA samples from these 2 patients were negative. Of 51 spesolimab treated patients, 50 patients were ADA evaluable with available ADA assessments pre- and post-baseline, including 1 patient who received placebo on Day 1 and an OL rescue spesolimab after Day 8. All 50 ADA-evaluable patients were ADA-negative at baseline.

The incidence rate of ADA+ patients observed in 1368-0013 is comparable with that from the proof-of-concept trial 1368-0011. Overall, 23 (46%) of the 50 ADA-evaluable and spesolimab-treated patients were ADA positive after treatment, and 27 patients (54%) were ADA negative through the trial duration. Twelve patients (24%) had a maximum titer of greater than 4000. The majority (87%) of the ADA-positive patients (40% of total treated) were also NAb positive and the NAb status appeared to be associated with the titer value. All ADA samples with titer value greater than 4000 were NAb positive. For ADA samples with titer value less than 4000, some were neutralizing, while others were not. The lower the titer values, the more likely the ADA samples were NAb negative. In ADA-positive patients, ADA developed early with a median onset time of 2.3 weeks and reached maximum titer at a median time of 11.7 weeks. In approximately 75% of patients, maximum titer occurred at the last sample collected. The time to maximum ADA titer and the titer itself may be influenced by the duration of the trial and collection times. In NAb-positive patients, Nab was detected at a median onset time of 6.7 weeks. At the end of the trial (12 to 17 weeks after the first active dose), the ADA was resolved in 4 out of 23 ADA-positive patients. Nineteen (38% of total ADA evaluable) patients remained ADA positive, 18 (36%) patients remained NAb positive, and 12 (24%) patients had a titer greater than 4000.

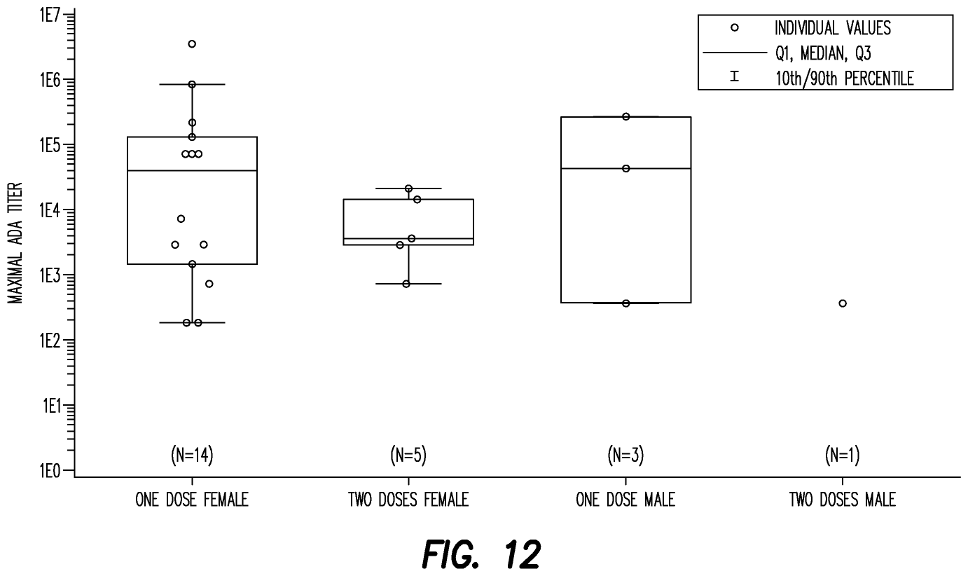

The ADA incidence rate was similar between patients treated with 1 dose of spesolimab and those who received 2 doses. However, the maximum ADA titers were observed to be lower in patients who received 2 doses of i.v. spesolimab within the first 8 days (Day 1 and Day 8) compared with patients who received only 1 i.v. dose (Table 10, FIG. 12).

TABLE 10
Trial 1368-0013: Incidence and kinetics of
spesolimab ADAs with i.v. spesolimab treatment

| | | | Patients who received | | Patients who received | |
|---|---|---|---|---|---|---|
| | Patients who received | | only 1 dose of | | 2 doses of | |
| | at least 1 dose of | | spesolimab i.v. | | spesolimab i.v. | |
| | spesolimab i.v. during | | (either on Day 1 | | (on both Day 1 | |
| | the trial¹ | | or Day 8)² | | and Day 8)² | |
| Total, N (%) | 50 | (100%) | 37 | (100%) | 12 | (100%) |
| Baseline³ ADA | 0 | (0%) | 0 | (0%) | 0 | (0%) |
| positive, N (%) | | | | | | |
| ADA negative⁴, N (%) | 27 | (54%) | 20 | (54%) | 6 | (50%) |
| Treatment-induced | 23 | (46%) | 17 | (46%) | 6 | (50%) |
| ADA positive⁵, N (%) | | | | | | |
| Transient positive⁶, N | 0 | (0%) | 0 | (0%) | 0 | (0%) |
| (%) | | | | | | |
| Potentially persistent | 23 | (46%) | 17 | (46%) | 6 | (50%) |
| positive⁷, N (%) | | | | | | |
| Persistent positive⁸, N | 0 | (0%) | 0 | (0%) | 0 | (0%) |
| (%) | | | | | | |
| Patients with | 12 | (24%) | 10 | (27%) | 2 | (17%) |
| maximum titer > 4000, | | | | | | |
| N (%) | | | | | | |
| Last sample ADA | 19 | (38%) | 14 | (38%) | 5 | (42%) |
| positive, N (%) | | | | | | |
| NAb positive⁹, N (%) | 20 | (40%) | 14 | (38%) | 6 | (50%) |
| Last sample NAb | 18 | (36%) | 13 | (35%) | 5 | (42%) |
| positive, N (%) | | | | | | |
| ADA onset [week] | 2.3 | (1.0 to 11.7) | 3.0 | (1.0 to 11.7) | 2.0 | (1.7 to 4.0) |
| (median, min to max) | | | | | | |
| Time of maximum titer | 11.7 | (2.0 to 16.7) | 11.6 | (2.0 to 16.3) | 12.9 | (2.0 to 16.7) |
| [week] (median, min to | | | | | | |
| max) | | | | | | |
| Maximum titer | 7200 | (180 to | 43200 | (180 to | 3240 | (360 to |
| (median, min to max) | | 3 600 000) | | 3 600 000) | | 21 600) |
| NAb onset [week] | 6.7 | (1.8 to 11.7) | 7.3 | (1.8 to 11.7) | 3.6 | (2 to 8.1) |
| (median, min to max) | | | | | | |

¹Also included a patient who did not receive any spesolimab on Days 1 or 8 but received a rescue treatment with spesolimab i.v. at Week 6.
²Regardless of whether a patient received rescue treatment with spesolimab i.v. after Day 8
³The last sample obtained before initiation of active spesolimab treatment^{4,5,6,7,8,9} As defined in Section 5.2.1 of the BLA

In addition, females appeared to have higher immunogenicity response. The ADA incidence rate was 58% and 24% in females and males, respectively. The percentage of patients with maximum titer of greater than 4000 was 30% in females vs. 12% in males (Table 11). Considering the small number of ADA positive male patients, there appear to be no significant differences in the maximum titers between genders.

TABLE 11
Trial 1368-0013: ADA incidence in female and male patients
with GPP after i.v. administration of spesolimab

| | Total | Female | Male |
|---|---|---|---|
| N (spesolimab treated | 50 | 33 | 17 |
| and evaluable) | | | |
| ADA incidence | 46% (23/50) | 58% (19/33) | 24% (4/17) |
| % of patient with | 24% (12/50) | 30% (10/33) | 12% (2/17) |
| maximum titer > 4000 | | | |

Relationship of Treatment-Emergent ADA/NAb to Efficacy

For the analysis, ADA titer groups were defined based on terciles of the maximal ADA titer across patients. The maximal ADA titer observed in the trial was used to distinguish 3 groups based on the maximum ADA titer ([≤33.3% percentile, 1440], [>33.3% percentile and 66.6% percentile], and [>66.6% percentile, 43200]). In addition, the ADA vs. efficacy analysis differentiated between NAb-negative and NAb-positive patients. For the patients included in the NAb-negative group, all samples were NAb-negative or ADA-negative. The patients included in the NAb-positive group had at least 1 sample that was NAb-positive.

Generally, in trial 1368-0013, the proportion of patients with a GPPGA pustulation subscore of 0 or a GPPGA total score of 0 or 1 over time was similar for ADA-negative and ADA-positive patients. When patients were grouped according to the terciles defined above, the response rates over time were generally similar across all 3 titer groups. Therefore, in general, this is also true when patients are categorized by having titer values below 4000 and above 4000. At Week 12, there appeared to be a drop in the response rate for patients with titer values greater than 43200. From the 8 patients with titer values greater than 43200, 4 had a reduction in the respective score while the other 4 did not. The drop in response rate was less strong with respect to the score itself, because 3 of the 4 patients had an increase by 1 point.

### Example 6

Spesolimab Treatment Improves CGI Scores Via the JDA Severity Index in Patients with GPP

In the Effisayil™ 1 trial, patients with a generalized pustular psoriasis (GPP) flare achieved pustular and skin clearance after receiving spesolimab. We report change from baseline in JDA severity index clinical global impression (CGI) scores at Week 1. Patients (N=53) were randomly assigned (2:1) to receive intravenous spesolimab 900 mg or placebo on Day 1 and followed for 12 weeks. In the spesolimab arm (n=31), 67.7% of patients saw improvement (scores 1-3), and 32.3% saw no improvement (scores 4-5). In the placebo arm (n=15), 53.3% of patients saw improvement, and 46.7% saw no improvement. A score of 1 (very much improved) was achieved by 76.2% of spesolimab improvers. In conclusion, spesolimab treatment improved JDA CGI scores in patients with a GPP flare compared with placebo.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method of treating generalized pustular psoriasis (GPP) flares in a patient, said method comprising administering to the patient two 900 mg intravenous (i.v.) doses of an anti-IL-36R antibody; wherein the second dose is administered less than 2 weeks after the first dose.

2. A method of treating GPP in a patient, said method comprising administering to the patient two 900 mg intravenous doses of an anti-IL-36R antibody; wherein the second dose is administered less than 2 weeks after the first dose.

3. A method of reducing or alleviating signs and symptoms of an acute phase flare-up of GPP in a patient, said method comprising administering to the patient two 900 mg intravenous doses of an anti-IL-36R antibody; wherein the second dose is administered less than 2 weeks after the first dose.

4. A method of reducing the severity and duration of GPP symptoms, said method comprising including administering to the patient two 900 mg intravenous doses of an anti-IL-36R antibody; wherein the second dose is administered less than 2 weeks after the first dose.

5. A method of treating a skin disorder associated with GPP, said method comprising administering to the patient two 900 mg intravenous doses of an anti-IL-36R antibody; wherein the second dose is administered less than 2 weeks after the first dose.

6. A method of preventing the recurrence of GPP flares in a patient, said method comprising administering to the patient two 900 mg intravenous doses of an anti-IL-36R antibody; wherein the second dose is administered less than 2 weeks after the first dose.

7. A method of reducing pain by at least 10% in a patient with GPP, said method comprising administering to the patient two 900 mg intravenous doses of an anti-IL-36R antibody; wherein the second dose is administered less than 2 weeks after the first dose.

8. A method of improving the quality of life by at least 10% in a patient with moderate to severe GPP symptoms, said method comprising administering to the patient two 900 mg intravenous doses of an anti-IL-36R antibody; wherein the second dose is administered less than 2 weeks after the first dose.

9. The method according to claim 1, wherein the patient has a GPP Physician Global Assessment (GPPGA) total score of ≥2.

10. The method according to claim 1, wherein the patient has a GPP Physician Global Assessment (GPPGA) total score of ≥2 and a GPPGA pustulation subscore of ≥2.

11. The method according to claim 1, wherein the patient has a GPP Physician Global Assessment (GPPGA) total score of ≥2 and a GPPGA pustulation subscore of ≥2 before the administration of the first i.v. dose.

12. The method according to claim 1, wherein the patient has a GPP Physician Global Assessment (GPPGA) total score of ≥2 and a GPPGA pustulation subscore of ≥2 after the administration of the first i.v. dose.

13. The method according to claim 1, wherein the patient has a GPP Physician Global Assessment (GPPGA) total score of ≥2 and a GPPGA pustulation subscore of ≥2 before and after the administration of the first i.v. dose.

14. The method according to claim 1, wherein the second dose is administered after 1 week but less than 2 weeks from the first dose.

15. A method of treating a GPP patient with a GPPGA pustulation subscore of ≥2, said method comprising the steps of: (a) administering to the patient a first 900 mg intravenous (I.V.) dose of an anti-IL-36R antibody; (b) assessing the GPPGA pustulation subscore of the patient and if the GPPGA pustulation subscore of ≥2 of the patient persists after 1 week from the first dose, then administering to the patient a second 900 mg (i.v.) dose of spesolimab less than 2 weeks after the first dose.

16. A method of treating a GPP patient with a GPP Physician Global Assessment (GPPGA) total score of ≥2, said method comprising the steps of: (a) administering to the patient a first 900 mg intravenous (i.v.) dose of an anti-IL-36R antibody; (b) assessing the GPPGA total score of the patient and if the GPPGA total score of ≥2 of the patient persists after 1 week from the first dose, then administering to the patient a second 900 mg (i.v.) dose of spesolimab less than 2 weeks after the first dose.

17. A method of treating a GPP patient with a GPP Physician Global Assessment (GPPGA) total score of ≥2 and a GPPGA pustulation subscore of ≥2, said method comprising the steps of: (a) administering to the patient a first 900 mg intravenous (I.V.) dose of an anti-IL-36R antibody; (b) assessing the GPPGA scores of the patient and if the GPPGA total score of ≥2 and the GPPGA pustulation subscore of 2 of the patient persist after 1 week from the first dose, then administering to the patient a second 900 mg (i.v.) dose of spesolimab less than 2 weeks after the first dose.

18. The method according to claim 1, wherein an optional third 900 mg i.v. dose of the anti-IL-36R antibody is administered at 2 to 12 weeks after the second i.v. dose.

19. The method according to claim 1, wherein the two-dose administration achieves one or more of the following results in:
(a) a Generalized Pustular Psoriasis Global Assessment (GPPGA) pustulation subscore of 0 indicating in one week after administering the second i.v. dose;
(b) a GPPGA total score of 0 or 1 in one week after administering the second i.v. dose; and/or (c) a lower maximum ADA titer at 2, 4, 6, 8, 10 or 12 weeks after administering the second i.v. dose.

20. The method of claim 19, wherein the results are maintained for up to and at least 12 weeks following the administration of the second i.v. dose.

21. The method according to claim 1, wherein the method further comprises administering to the patient a prophylactically effective amount of the anti-IL-36R antibody in one or more subcutaneous doses after the last i.v. dose administered.

22. The method according to claim 21, wherein each of the one or more subcutaneous doses comprises 150 mg, 225 mg, 300 mg, 450 mg, or 600 mg of said anti-IL-36R antibody.

23. The method according to claim 22, wherein 1, 2, 3 or more subcutaneous doses are administered to the patient and wherein a first subcutaneous dose is administered after the last intravenous dose.

24. The method according to claim 23, wherein a first subcutaneous dose is administered 2 to 8 weeks, 4 to 6 weeks, 2 weeks, 4 weeks, 6 weeks or 8 weeks, 12 weeks, 16 weeks, 20 weeks after the last intravenous dose, and subsequent subcutaneous doses are administered at 2, 4, 6, 8, 10 or 12 weeks intervals after the first subcutaneous dose.

25. The method according to claim 1, wherein the patient remains in clinical remission as measured by a GPPGA total score of 0 or 1 for at least 12, 24, 36, 48, 60 or 72 weeks following the last i.v. or subcutaneous dose.

26. The method according to claim 1, wherein the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

27. The method according to claim 1, wherein the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

28. The method according to claim 1, wherein the anti-IL-36R antibody comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

29. The method according to claim 1, wherein the anti-IL-36R antibody comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

30. The method according to claim 1, wherein the anti-IL-36R antibody is spesolimab.
